(19) United States
(12) Patent Application Publication
(10) Pub. No.: US 2022/0160377 A1
(43) Pub. Date: May 26, 2022

(54) SYSTEM FOR RESTORING PATENCY ACROSS AN OBSTRUCTION

(71) Applicant: RampTech, LLC, Honolulu, HI (US)

(72) Inventors: John Hugh Rundback, Bridghampton, NY (US); Peter A. Schneider, Honolulu, HI (US); Michael J. Horzewski, San Jose, CA (US)

(21) Appl. No.: 17/103,032

(22) Filed: Nov. 24, 2020

(51) Int. Cl. A61B 17/22; A61B 17/34

(52) U.S. Cl. CPC A61B 17/22; A61M 25/007; A61B 17/3423

(57) ABSTRACT

Disclosed are methods and systems for restoring patency across a vascular or non-vascular occlusion. The system may include a retrograde catheter, having a proximal end, a distal end, a first central lumen and a first side port spaced proximally apart from the distal end, the first central lumen extending at least as far as the first side port. An antegrade catheter is also provided, having a proximal end, a distal end, a second central lumen in communication with a second side port spaced proximally apart from the distal end. The catheters may have complementary surface configurations to facilitate alignment of the first and second side ports, so that a wire may be passed out of one of the side ports and into the other side port.

### FIELD OF THE INVENTION

Described are systems and methods for the treatment of revascularization and recanalization of vascular and non-vascular targets. The systems and methods can be used in vascular and non-vascular applications, such as the treatment of chronic limb threatening ischemia or critical limb ischemia, recanalization and revascularization involving chronic total occlusions, lower leg and pedal occlusions, upper leg and iliac arterial occlusions, venous occlusions, and other targets accessed from first and second directions.

### BACKGROUND OF THE INVENTION

For patients with peripheral artery disease (PAD), there is increasingly recognized clinical benefit to accessing the smaller peripheral and pedal arteries in a retrograde fashion to allow successful catheter-based recanalization and revascularization of occluded arterial segments in the lower extremities. In a recent analysis of the multicenter Vascular Quality Initiative (VQI) Registry, ⅓ of PAD procedures included the use of retrograde pedal access (Perry M, Callas P W, Alef M J, Bertges D J, Outcomes of Peripheral Vascular Interventions via Retrograde Pedal Access for Chronic Limb-Threatening Ischemia in a Multicenter Registry. J Endovasc Ther. 2020 April; 27(2):205-210. doi: 10.1177/1526602820908056. Epub 2020 Feb. 19).

The principle limitation of small vessel (e.g. pedal, radial) arterial access is difficulty with successful needle cannulation and introduction through the needle of a guide wire for subsequent intervention. This challenge is due to common characteristics of the peripheral artery at the desired puncture site including small diameter, mobility, and dense calcification. Failed needle cannulation may result in bleeding, spasm, arterial thrombosis, nerve compression, compartment syndrome and worsening ischemia, particularly when multiple punctures are attempted to allow wire insertion. While data is scarce regarding the failure rate of attempted retrograde arterial puncture and wire insertion, this is expected to be high as a result of unfavorable anatomic characteristics as well as variable operator experience with this technique (Hernan A, Bazan L L, Donovan M, et al. Retrograde pedal access for patients with critical limb ischemia J Vasc Surg. 2014; 60:375-382). Initial and one-time engagement of and needle anchoring in calcified, mobile, and small peripheral arteries would be expected to increase the success rate and clinical utility of this strategy as well as reduce potential complications. The RAMP′ needle is purpose-built to specifically address the limitations and risks of current needle access systems in securing retrograde pedal (and radial) access in both routine and anatomically challenging scenarios.

There is a growing population of patients with chronic limb threatening ischemia (CTLI, or CLI, critical limb ischemia) due to an aging population and increasing prevalence of diabetes, chronic kidney disease, and metabolic syndrome. Endovascular catheter-based therapy has become first line approach for the treatment of many patients with CLI due to extensive comorbidities and a lack of both suitable surgical conduit and distal target vessels for surgical bypass. These patients are often characterized by long segment multivessel densely calcified tibiopedal arterial occlusions which are a major limitation to successful revascularization and contributes to high rates of major amputation in this population. In recent years, the use of pedal access and retrograde recanalization in selected centers has increased procedural success allowing successful recanalization and revascularization of anatomically complex peripheral arterial occlusions and amputation prevention. However, the skill set and tools necessary for successful pedal access procedures is not uniformly available.

In many if not most cases, the retrograde wire passes subintimally (within the vessel wall) across occluded arterial segments before successfully entering the patent arterial lumen cephalad to the obstruction, where the wire is then captured by some means and exteriorized at a separate femoral access for through and through wire control. These procedures have been called PIER (Percutaneous Intentional Extraluminal Revascularization), CART (Combined Antegrade and Retrograde Transluminal revascularization), and SAFARI (Subintimal Antegrade Flossing with Antegrade and Retrograde Intervention). However, more recently, the common and accepted terminology for this strategy has been called “Rendezvous” procedures” (Banerjee S, Shishehbor M H, Mustapha J A, Armstrong E J, Ansari M, Rundback J H, Fisher B, Peña C S, Brilakis E S, Lee A C, Parikh S J. A Percutaneous Crossing Algorithm for Femoropopliteal and Tibial Artery Chronic Total Occlusions (PCTO Algorithm). J Invasive Cardiol. 2019 April; 31(4):111-119.

Despite this, failure rates of this technique remain high due to an inability to successfully pass wires introduced via retrograde pedal access into patent proximal arterial segments (Bazan H A, Le L, Donovan M, Sidhom T, Smith T A, Sternbergh W C 3rd. Retrograde pedal access for patients with critical limb ischemia. J Vasc Surg. 2014 August; 60(2):375-81. doi: 10.1016/j.jvs.2014.02.038. Epub 2014 Mar. 18). Instead, wires introduced from both antegrade and retrograde directions enter separate parallel subintimal channels that do not freely pass into either proximal or distal patent segments. If continuity cannot be established between patent arterial segments proximal (above) and distal (below) the arterial occlusion, revascularization is not possible.

There are several existing technical strategies to attempt to achieve successful through and through wire access (from patent proximal to patent distal arterial lumens), none of which are specifically designed for this purpose. A common strategy is the use of “Reentry” devices (Cordis Outback device, Medtronic Pioneer and Enteer catheters, Boston Scientific Off-Road catheter). These devices were engineered for and approved for reentry from subintimal to intraluminal channels in the larger femoral vessels, and NOT for reentry into separate parallel subintimal channels. The caliber of these devices is generally less well suited for use in tibiopedal occlusions, and for devices with side exiting needles (Outback and Pioneer) the distance traversed during the “throw” (forward advancement) of the side needle is longer than desired and may result in excessive bleeding or other injury both compromising success and increasing complications. To improve success, operators have placed snare devices or balloons in the retrograde channel as a target for puncture of the reentry device. However, since these represent separate and disparate devices with completely different intended purposes, the antegrade reentry catheter and retrograde device are often not well aligned and are difficult to control and position. The technical challenges of this technique can be cumbersome and dramatically limit the clinical applicability and physician acceptance of reentry devices for Rendezvous procedures. The RAMP Rendezvous catheter set represents a distinct purpose-built solution to Rendezvous procedures using specially designed integrated and aligned catheter systems for successful through and through wire passage and endovascular therapy.

For achieving passage of a guide wire between separate parallel subintimal channels or subintimal to intraluminal channels with the RAMP rendezvous catheter set, a specifically designed rendezvous guide wire can be employed with a piercing end to facilitate passage of the guide wire through any tissue that may be between the RAMP rendezvous catheter set.

### SUMMARY OF THE INVENTION

In accordance with one aspect of the present invention, there is provided a system useable for performing a therapeutic task at a location within the body of a human or animal subject, such system comprising: a) needle, b) retrograde catheter, c) antegrade catheter, and d) rendezvous guide wire. The system is capable of overcoming the current limitations in procedures where it is desirable to recanalize a vascular or non-vascular obstruction/occlusion where access to both sides of an occlusion (e.g. retrograde and antegrade) is achievable. The system and/or any of its components, individually or in combination, can be used in vascular and non-vascular applications, including but not limited to, lower leg and pedal occlusions, upper leg and iliac arterial occlusions, venous occlusions, other targets with potential antegrade and retrograde access, around and/or through an occlusion(s) (e.g. hepatobiliary, urinary tract, gastrointestinal tract occlusions).

The system includes a needle that provides for improved access to and stability within a target vessel, including smaller and/or diseased/calcified/mobile vessels. A retrograde catheter capable of bypassing/traversing an occlusion and providing an exit port for a rendezvous guide wire with complimentary features to an antegrade catheter. An antegrade catheter with complementary features to the retrograde catheter and capable of receiving a rendezvous guide wire. A rendezvous guide wire with the ability to ease the passage of the rendezvous guide wire though any tissue between the retrograde catheter exit port and a vessel lumen. In addition, a microcatheter may be included to maintain retrograde vascular access.

Still further in accordance with the invention, there is provided a needle. The needle has a lance tip, capable of penetrating smaller and/or diseased/calcified/mobile vessels as well as providing improved stability once in position, a shaft section with a lumen and distal lumen opening through which various guide wires may be moved, and a hub section for insertion of various guide wires, injection of fluids (e.g. contrast media), connection to a syringe or other device, etc. The needle may include other features such as a transition section between the lance tip and shaft section, various radiopaque, tactile, and/or visual indicators or markers, a bumper to assist in positioning the needle such that the distal lumen opening is properly located within the vessel, and a ramp to assist in easing a guide wire out the distal lumen opening.

Still further in accordance with the invention, there is provided a retrograde catheter. The retrograde catheter has features for bypassing an occlusion by creating a directional dissection plane in the vessel wall, such as subintimal, intimal, intramural (medial), subadventitial, through the occlusion itself, within the true lumen, through the occlusion, or a combination thereof. These features include a distal region configured to enable dissection through a vessel wall (e.g. between the intima and media) and/or occlusion; examples include round or non-round shaped such as a flattened, spatulated, crescent shape, edgy, etc.; to facilitate orientation within the vessel; alignment with an antegrade catheter; and passage across occluded segments allowing blunt or cutting dissection similar to surgical elevators (in effect, a remotely introduced endovascular surgical tool). The retrograde catheter provides a lumen for a guide wire for guidance of the retrograde catheter through the vessel or desired body region. The retrograde catheter provides a lumen for delivering a rendezvous guide wire to and out of an exit port. The guiding lumen and rendezvous guide wire lumen may be the same or different lumens or a combination thereof. The ramp section can contain a ramp for guiding the rendezvous guide wire out of the exit port, which may be configured to achieve a desired exit angle to improve tissue penetration, and entry into a receiving side port/window of an accompanying antegrade catheter. At least a portion of the ramp section and/or distal shaft may be configured to align, in one or more of lateral, longitudinal, and radial directions, the exit port and receiving window of retrograde and antegrade catheters, for example by having a particular complimentary shape(s), alignment marker(s), active component(s), hoop(s), balloon(s), extension wire(s), etc. Further, at least a portion of the ramp section and/or distal shaft may be constructed to urge, move, or orient the exit port towards the window section, such as by having a stepped, curved, shaped section, and/or mechanically moveable element(s). The retrograde catheter and shaft can be constructed in various configurations to achieve the desired result. These include but are not limited to a mini-rail, side-by-side mini-rail, over the wire, moveable ramp (fixed or moveable), and a side-by-side over the wire configuration. Depending on the shaft configuration, various hubs may be employed in the proximal region to enable access to the lumens and any moveable features. In addition, the retrograde catheter can also be used in combination with any of the other system components or with conventional devices (e.g. needles, guide wires, and microcatheters) in treatments for vascular and other parts of the body.

Still further in accordance with the invention, there is provided an antegrade catheter. The antegrade catheter has features for receiving a (rendezvous) guide wire and aligning a window section with an exit port section of a retrograde catheter. The antegrade catheter provides a lumen for a guide wire for guidance of the antegrade catheter through the vessel or desired body region. The antegrade catheter provides a window and a lumen for receiving a rendezvous guide wire. The guiding lumen and rendezvous guide wire lumen may be the same or different lumens or a combination thereof. The window section can be reinforced to receive the rendezvous guide wire without damage, such as when the rendezvous guide wire has a piercing tip, and may be configured to achieve a desired entry angle of the rendezvous guide wire from a retrograde catheter. At least a portion of the window section and/or distal shaft may be configured to align and/or position, in one or more of lateral, longitudinal, and radial directions, the window and exit port of antegrade and retrograde catheters, for example by having a particular complimentary shape(s), alignment marker(s), active component(s), hoop(s), balloon(s), extension wire(s), etc. Further, at least a portion of the window section and/or distal shaft may be constructed to urge, move, or orient the exit port towards the window section, such as by having a stepped, curved, shaped section, and/or mechanically moveable element(s). The antegrade catheter and shaft can be constructed in various configurations to achieve the desired result. These include but are not limited to a mini-rail, side-by-side mini-rail, over the wire, moveable ramp (fixed or moveable), multilumen, and a side-by-side over the wire configuration. Depending on the shaft configuration, various hubs may be employed in the proximal region to enable access to the lumens and any moveable features. In addition, the antegrade catheter can also be used in combination with any of the other system components or with conventional devices (e.g. needles, guide wires, and microcatheters) in treatments for vascular and other parts of the body.

Still further in accordance with the invention, there is provided a hoop(s) or loop(s) that can be extended from the antegrade catheter and used to capture the rendezvous guide wire, retract/retrieve the rendezvous guide wire, and/or to enable pulling or guiding the rendezvous guide wire through the window. The hoop can extend from the antegrade catheter with the hoop proximally, distally, or from within the window or window region. The hoop can also be used to capture the rendezvous guide wire and then by retracting the antegrade catheter with hoop, bring the rendezvous guide wire back out of the antegrade access site to complete the rendezvous guide wire positioning within the patient. The hoop (with or without a microcatheter or other means to engage the hoop capture mechanism) can also be used to separately capture and retract the rendezvous guide wire within the antegrade catheter (through the lumen of the antegrade catheter) until it is brought out of the antegrade access site to complete the rendezvous guide wire positioning.

Still further in accordance with the invention, there is provided a microcatheter for use with a retrograde guide wire, or other guide wires. The microcatheter can be used maintain vascular access when removing a guide wire from the vessel, such as when removing an initial short guide wire used through the needle to achieve vascular access and then passing the retrograde guide wire through the microcatheter into the target vessel, or to facilitate engagement of a hoop capture mechanism. The microcatheter may also serve as a dilator to further open the connection created between the antegrade and retrograde lumens and to facilitate positioning of subsequent wires, catheters, devices, etc. Similarly, there may be variations on the microcatheter to further increase or modify the connection created between the antegrade and retrograde lumens.

Still further in accordance with the invention, there is provided a rendezvous guide wire. The rendezvous guide wire may include features to improve movement of the rendezvous guide wire from the retrograde catheter into the antegrade catheter and/or penetrating tissue. The rendezvous guide wire has a distal end region and a proximal end region. A core wire extends relatively the length of the rendezvous guide wire and may be made from one or more elements which may vary in material, such as stainless steel and Nitinol. The end regions may be tapered to achieve the desired flexibility and pushability. The distal end region core wire element may have a taper or angulation to ease the transition from a retrograde catheter to an antegrade catheter. The distal end region includes coils and/or a jacket (e.g. polymer coating) over a core wire element. At least a portion of the coil(s), polymer, core wire, or marker may be radiopaque. Coil(s) may be of varying materials, e.g. stainless steel, Platinum, Platinum-Iridium, etc., and have varying degrees of coil spacing and diameters of both the wire used to wind the coil and the coil itself. The distal tip may have a piercing element to improve penetrating tissue or be atraumatic. The proximal end region core wire element may have a taper to add flexibility. The proximal end region may have coils and/or a jacket (e.g. polymer coating) over a core wire element. At least a portion of the coil(s), polymer, core wire, or marker may be radiopaque. Coil(s) may be of varying materials, e.g. stainless steel, Platinum, Platinum-Iridium, etc., and have varying degrees of coil spacing and diameters of both the wire used to wind the coil and the coil itself. The proximal tip may have a piercing element to improve penetrating tissue or be atraumatic. All or a portion of the rendezvous guide wire may be coated to improve movement through one or both of the retrograde and antegrade catheters, and/or tissue.

Still further in accordance with the invention, there is provided in one or more embodiments, the antegrade and retrograde catheters aligning within the occluded segment or occlusion, cranial to (above) the occluded segment (as illustrated), caudal to (below) the occluded segment, or medial and lateral, or anterior and posterior. For use in this invention, we will use antegrade and retrograde to describe two different directions. It is understood that the present invention covers access or catheter introduction from more than one direction, not just with respect to antegrade and retrograde. As such, an antegrade catheter may have any or all of the features, components, and design aspects to partially or wholly bypass an occlusion and deliver a rendezvous guide wire as previously described for the retrograde catheter, and a retrograde catheter may have any or all of the features, components, and design aspects to align with the antegrade catheter and receive a rendezvous guide wire that is passed through the catheters in any location with respect to the occlusion.

These components can be used as a complete system, individually, in combinations, and/or with other needles, guide wires, catheters, and vascular and non-vascular devices.

Still further in accordance with the invention, there is provided a method for performing a therapeutic task at a location within the body of a human or animal subject, such method comprising the steps of: a) obtaining retrograde access to a target vessel; by orienting the needle using one or more of visual (including ultrasound or fluoroscopy guided) and tactile indicators, references, and surgical access; advancing the needle into the tissue and having the lance tip penetrate the vessel wall; positioning the needle distal lumen opening within the lumen of the vessel; inserting a retrograde guide wire though the needle lumen and into the vessel; and retracting the needle; leaving the retrograde guide wire in place; b) introducing a retrograde catheter and crossing the occlusion, by inserting the distal portion of a retrograde catheter over the retrograde guide wire, advancing the retrograde catheter along the retrograde guide wire to the region of the occlusion, rotationally orienting the retrograde catheter, advancing the retrograde catheter through the tissue and/or occlusion (e.g. by blunt or cutting dissection creating a directional dissection plane, with or without manipulation of the retrograde guide wire), c) obtain antegrade access to the target vessel, by advancing a needle into the tissue and vessel or through surgical access, positioning the needle distal opening within the lumen of the vessel, inserting an antegrade guide wire though the needle and into the vessel, and retracting the needle, leaving the antegrade guide wire in place; d) introducing an antegrade catheter, by loading the distal portion of an antegrade catheter onto the antegrade guide wire, advancing the antegrade catheter along the antegrade guide wire to the region of the occlusion; e) orienting the antegrade catheter with the retrograde catheter, by rotating and advancing the antegrade catheter and using complimentary surfaces and markers to align the retrograde catheter exit port and antegrade catheter window in rotational, lateral, and longitudinal directions; f) obtaining guide wire rendezvous, by inserting the distal end region of the rendezvous guide wire into the rendezvous guide wire lumen of the retrograde catheter, advancing the rendezvous guide wire across the ramp and out the exit port of the retrograde catheter and into the window of the antegrade catheter, continuing to advance the rendezvous guide wire until at least a portion of the distal end region of the rendezvous guide wire exits the antegrade catheter; g) removing the antegrade catheter from the body by retracting it over and off of the rendezvous guide wire; h) removing the retrograde catheter from the body by retracting it over and off of the rendezvous guide wire; i) performing a revascularization procedure to improve blood flow through the region of the occlusion by inserting a device(s) over the rendezvous guide wire and conducting the revascularization procedure; j) removing the revascularization device(s) and rendezvous guide wire from the body; k) and closing the retrograde and antegrade access sites.

Still further in accordance with the current invention, there is provided a method of passing a microcatheter/dilator over a guide wire to enlarge the passageway through/around/past an occlusion.

Still further in accordance with the invention, there is provided a method when needle access to the vessel is obtained, inserting a guide wire through the needle and into the vessel lumen, retracting the needle, leaving the guide wire in place, advancing a microcatheter over the guide wire establishing access to the vessel lumen with the microcatheter, removing the guide wire, advancing a retrograde guide wire through the microcatheter and into the vessel lumen, and removing the microcatheter.

Still further in accordance with the invention, there is provided a method where a guide wire is used to gain access to the vessel lumen.

Still further in accordance with the invention, there is provided a method when an antegrade catheter window is in relative alignment with the retrograde catheter exit port, employing passive and/or active features (e.g. catheter(s) shape, balloon(s), wire(s)) to move the antegrade catheter window and retrograde catheter exit port together. In one example of a passive feature, the distal region of the antegrade catheter has a bend, or curvature, or offset where the antegrade catheter can be rotated such that the tip or distal region is positioned against the vessel wall and this orients the window in the relative opposite direction moving the window in close proximity to the retrograde catheter exit port. Examples of active features include, the antegrade catheter can have a balloon or wire(s) extending from the relative opposite side of the antegrade catheter as the window, such that when the balloon is inflated or wire(s) deployed, the window is moved in the relative opposite direction in close proximity to the retrograde catheter exit port.

Still further in accordance with the invention, there is provided a method where a hoop(s) or loop(s) is extended from the antegrade catheter and used to capture the rendezvous guide wire, retract/retrieve the rendezvous guide wire, and/or to enable pulling or guiding the rendezvous guide wire through the window. The hoop can extend from antegrade catheter with the hoop proximally, distally, or from within the window or window region. The hoop can also be used to capture the rendezvous guide wire and then by retracting the antegrade catheter with hoop, bring the rendezvous guide wire back out of the antegrade access site to complete the rendezvous guide wire positioning within the patient.

Still further in accordance with the invention, there is provided a method when an antegrade catheter distal region is near the retrograde catheter exit port, the distal region of an antegrade catheter may be rotated and aligned such that it is in contact with or pointing towards the vessel wall at the relative location of the retrograde catheter exit port. The antegrade guide wire is retracted and/or positioned to achieve the desired deflection of the antegrade distal region to engage the distal tip and/or dissection feature against the vessel wall and then manipulating (e.g. moving longitudinally, laterally, a combination thereof) the distal tip and/or dissection feature to disrupt tissue (e.g. intima) between the retrograde catheter exit port and the antegrade catheter distal tip and/or dissection feature.

Still further in accordance with the invention, there is provided a method for piercing tissue that may be between the retrograde catheter exit port and vessel lumen and/or antegrade catheter window. This can be achieved by inserting the rendezvous guide wire end with or without a piercing feature into the rendezvous guide wire lumen of the retrograde catheter, advancing the rendezvous guide wire out the exit port and through the tissue, into the vessel lumen and/or antegrade catheter window. An example is having the proximal end region of a rendezvous guide wire with a tapered core, angulation, a radiopaque coil, and a piercing tip; inserting the proximal end region of the rendezvous guide wire into the rendezvous guide wire lumen of the retrograde catheter; advancing the rendezvous guide wire until the piercing tip is near or at the exit port; ensuring orientation of the exit port with the vessel lumen and/or with the antegrade catheter window; advancing the piercing tip through the tissue; retracting the rendezvous guide wire from the retrograde catheter; inserting and advancing the distal end of the rendezvous guide wire through the retrograde catheter rendezvous guide wire lumen, out the exit port, through the pierced tissue, and into the window of the antegrade catheter. A further example is having the distal end region of a rendezvous guide wire with a tapered core, a radiopaque coil, and a piercing tip. Inserting the distal end region of the rendezvous guide wire into the rendezvous guide wire lumen of the retrograde catheter, advancing the rendezvous guide wire until the piercing tip is near or at the exit port, ensuring orientation of the exit port with the vessel lumen and/or with the antegrade catheter window, advancing the piercing tip through the tissue, continuing to advance the rendezvous guide wire into the antegrade catheter window and through the antegrade catheter until the distal end of the rendezvous guide wire exits the antegrade catheter.

Still further in accordance with the invention, there is provided a method for having the antegrade and retrograde catheters aligning within the occluded segment or occlusion, cranial to (above) the occluded segment, caudal to (below) the occluded segment, or medial and lateral, or anterior and posterior. As such, an antegrade catheter may have any or all of the features, components, and design aspects to partially or wholly cross an occlusion previously described for the retrograde catheter, and a retrograde catheter may have any or all of the features, components, and design aspects to align with the antegrade catheter enabling a rendezvous guide wire to be passed through the catheters in any location with respect to the occlusion.

Still further in accordance with the invention, there is provided a method for having the antegrade and retrograde catheters aligning within the occluded segment or occlusion, cranial to (above) the occluded segment, caudal to (below) the occluded segment, or medial and lateral, or anterior and posterior. Moving the antegrade and retrograde catheters individually or in combination with each other, either simultaneously or one-at-a-time, in the same direction or the opposite direction (inserting or retracting the catheters) to create a channel through the occlusion. Advancing a guide wire or catheter through the channel.

Further aspects, embodiments, variations, details, elements, and examples of the present inventions will be understood by those of skill in the relevant art from the accompanying drawings and the following detailed description.

### BRIEF DESCRIPTION OF THE DRAWINGS

A general architecture that implements the various features of the invention will now be described with reference to the drawings. The drawings and the associated descriptions are provided to illustrate some but not all embodiments or examples of the invention and do not limit the scope of the claimed inventions in any way. Throughout the drawings, reference numbers are re-used to indicate correspondence between referenced elements.

FIG. 1 illustrates an embodiment of the RampTech System.

FIGS. 2A-B illustrate an embodiment of the needle in side-elevational view (2A) and side-elevational cross-sectional view (2B).

FIGS. 3A-B illustrate additional embodiments of the needle in side-elevational view.

FIG. 3C illustrates a microcatheter in side-elevational view.

FIG. 4A illustrates an embodiment of a mini-rail retrograde catheter in side-elevational view with a retrograde guide wire.

FIG. 4B illustrates an embodiment of a cross-section through a portion of the rendezvous guide wire shaft.

FIG. 4C illustrates an embodiment of a cross-section through a portion of the min-rail shaft.

FIG. 4D illustrates an embodiment of the ramp section in side-elevational cross-sectional view.

FIG. 4E illustrates an alternate embodiment of a side-elevational cross-sectional view of the ramp section.

FIG. 4F illustrates an alternate embodiment of a side-elevational cross-sectional view of the mini-rail shaft and ramp section.

FIG. 5A illustrates an embodiment of an exit port radiopaque marker in side-elevational view.

FIG. 5B illustrates the embodiment of an exit port radiopaque marker of FIG. 5A rotated by 90 degrees (top view) along the longitudinal axis of the retrograde catheter.

FIGS. 5C-E illustrate additional embodiments of an exit port radiopaque marker in side-elevational view.

FIG. 6 illustrates an embodiment of an over the wire retrograde catheter in side-elevational cross-sectional view.

FIG. 7 illustrates an embodiment of a single lumen over the wire retrograde catheter with a moveable ramp in side-elevational cross-sectional view.

FIG. 8A illustrates an additional embodiment of a single guide wire lumen over the wire retrograde catheter with a manually actuated moveable ramp in side-elevational cross-sectional view.

FIG. 8B illustrates a cross-sectional view of the catheter in FIG. 8A taken proximal to the manually actuated moveable ramp.

FIG. 9A illustrates an embodiment of a single lumen antegrade catheter in side-elevational view.

FIG. 9B illustrates a side-elevational cross-sectional view of the single lumen antegrade catheter of FIG. 9A.

FIG. 9C-D illustrates embodiments of a cross-sectional view of the single lumen antegrade catheter of FIG. 9A.

FIG. 10 illustrates a side-elevational view of the single lumen antegrade catheter.

FIG. 11A illustrates an embodiment of a mini-rail antegrade catheter and antegrade guide wire in side-elevational view.

FIG. 11B illustrates the mini-rail antegrade catheter of FIG. 11A in side-elevational cross-sectional view.

FIG. 11C illustrates the mini-rail antegrade catheter of FIG. 11A in cross-section through the mini-rail shaft.

FIG. 12 illustrates an embodiment of a multilumen over the wire antegrade catheter in side-elevational cross-sectional view.

FIG. 13A illustrates embodiments of a mini-rail retrograde catheter and a single lumen antegrade catheter in position for rendezvous guide wire passage in side-elevational view.

FIGS. 13B-D illustrate examples of cross-sections of embodiments of the catheters in FIG. 13A.

FIG. 14 illustrates an embodiment of a mini-rail retrograde catheter and a portion of a single lumen antegrade catheter with a step feature aligned for a rendezvous guide wire passage in side-elevational view.

FIG. 15 illustrates an embodiment of a portion of an antegrade catheter with a step and a recess in partial (rotated) 3-D view.

FIG. 16 illustrates an embodiment of an over the wire retrograde (OTWR) catheter with an offset section of OTWR multilumen shaft in the region of the exit port in side-elevational view.

FIG. 17A illustrates an embodiment of a balloon on an antegrade catheter in side-elevational view.

FIG. 17B illustrates a cross-section through an antegrade catheter with balloon of FIG. 17A, the inflated balloon, the vessel, and a retrograde catheter.

FIG. 18A illustrates an embodiment of extension wires on an antegrade catheter in side-elevational view.

FIG. 18B illustrates a cross-section through an antegrade catheter with extension wires of FIG. 18A, the extended extension wires, the vessel, and a retrograde catheter.

FIG. 19A illustrates an embodiment of a hoop on a retrograde catheter in side-elevational view.

FIG. 19B illustrates a cross-section through a retrograde catheter with hoop of FIG. 19A and an antegrade catheter captured within the hoop.

FIG. 19C illustrates an embodiment of an antegrade catheter with hoop in side-elevational view.

FIGS. 19D-E illustrate an embodiment of an antegrade catheter with hoop and capturing a rendezvous guide wire in the hoop in side-elevational view.

FIG. 20 illustrates an embodiment of a rendezvous guide wire in side-elevational view.

FIG. 21A illustrates an embodiment of a mini-rail retrograde catheter in bottom-up view with a retrograde guide wire.

FIGS. 21B-D illustrate cross-sections through the mini-rail retrograde catheter of FIG. 21A.

FIG. 22 illustrates a step in a method for identifying a target retrograde vessel using ultrasound in side-elevational view.

FIG. 23 illustrates a step in a method for determining a location for accessing a target vessel in cross-sectional view with the needle in side-elevational view.

FIG. 24 illustrates a step in a method of a portion of a patient's leg in cross-sectional view and the needle positioned through the tissue and having the lance portion of the needle within a vessel in a closer side-elevational view.

FIG. 25 illustrates a step in a method of a portion of a patient's leg in cross-sectional view with the needle positioned within a vessel in a closer side-elevational view.

FIG. 26 illustrates a step in a method of a portion of a patient's leg in cross-sectional view with a retrograde guide wire placed through the needle in side-elevational view.

FIG. 27 illustrates a step in a method of a portion of a patient's leg in cross-sectional view with a retrograde guide wire within a patient's target vessel after removing the needle in side-elevational view.

FIG. 28 illustrates a step in a method of a portion of a patient's leg in cross-sectional view with a retrograde guide wire and a retrograde catheter distal region in side-elevational view within the target vessel lumen.

FIG. 29 illustrates a step in a method of a portion of a patient's leg in cross-sectional view with the retrograde catheter in side-elevational view oriented and traversing a target occlusion (the retrograde catheter may traverse within lumen or in subintimal space [as illustrated]).

FIG. 30 illustrates a step in a method of a portion of a patient's leg cross-sectional view with the retrograde catheter in side-elevational view oriented and having crossed a target occlusion (antegrade and retrograde catheters may align within occluded segment, cranial to (above) occluded segment, or caudal to (below) occluded segment, or medial and lateral, or anterior and posterior).

FIG. 31 illustrates a step in a method of a portion of a patient's target vessel in cross-sectional view with a retrograde catheter and a rendezvous guide wire in side-elevational view piercing the vessel wall.

FIG. 32 illustrates a step in a method with an introducer sheath placed in the target antegrade vessel in top-down view.

FIG. 33 illustrates a step in a method of a portion of a patient's target vessel with an antegrade catheter micro-dissecting the vessel wall in cross-sectional view.

FIG. 34 illustrates a step in a method of a portion of a patient's target vessel in cross-sectional view with an antegrade catheter and a retrograde catheter in position in side-elevational view.

FIG. 35 illustrates a step in a method of a portion of a patient's target vessel of both an antegrade catheter and a retrograde catheter with a rendezvous guide wire exiting a retrograde catheter and entering an antegrade catheter in cross-sectional view.

FIG. 36 illustrates a step in a method with the long rendezvous guide wire extending from out of a retrograde catheter hub and out of an antegrade catheter hub in top-down view.

FIG. 37 illustrates a step in a method with the rendezvous guide wire extending from out of a retrograde access location and an antegrade sheath.

FIG. 38 illustrates a step in a method of a portion of a patient's target vessel in cross-sectional view with a balloon angioplasty catheter positioned on the rendezvous guide wire in side-elevational view across an occlusion as an example of a subsequent treatment modality.

### DETAILED DESCRIPTION OF THE PREFERRED EMBODIMENT

The inventions disclosed herein may be embodied in other specific forms without departing from its spirit or essential characteristics. The described embodiments are to be considered in all respects only as illustrative and not restrictive. The scope of the inventions is therefore indicated by the appended claims rather than the foregoing description. All changes that come within the meaning and range of equivalency of the claims are to be embraced within their scope.

The RampTech System 10 can be used as part of a procedure where it is desirable to recanalize a vascular or non-vascular obstruction/occlusion 2300 where access to both sides of an occlusion 2300 (e.g. retrograde and antegrade) is achievable. The RampTech System 10 will be described for treating a lower limb chronic total occlusion by creating a directional dissection plane in the vessel 2200 wall around an occlusion, however, the RampTech System 10 and/or any of its components, individually or in combination, can be used in vascular and non-vascular applications, including but not limited to, lower leg and pedal occlusions, upper leg and iliac arterial occlusions, venous occlusions, other targets with potential antegrade and retrograde access, around and/or through an occlusion(s). Obstruction and occlusion are used interchangeably throughout.

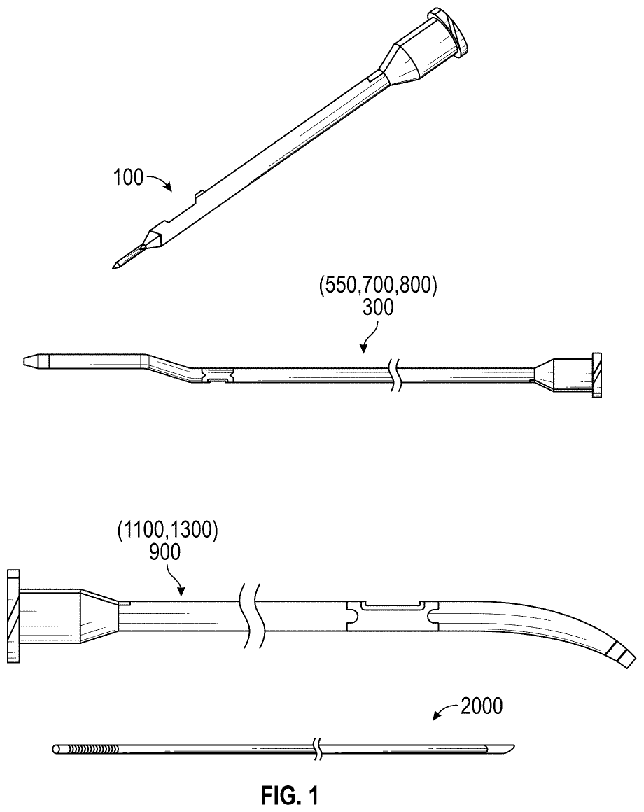

FIG. 1 illustrates an embodiment a RampTech System 10, including a needle 100, a retrograde catheter 300 (550, 700, 800), an antegrade catheter 900 (1100, 1300), and a rendezvous guide wire 2000 that can be used as a system or individually (e.g. with other guide wires, needles, and/or catheters) to enable vessel 2200 recanalization. It will be understood that though the RampTech System 10, its components, and methods described herein are described primarily with reference to lower extremity vascular procedures creating a directional dissection plane in a vessel 2200 wall around an occlusion 2300, they can also be used through an occlusion 2300 and/or within the lumen of a vessel 2200 as well as in treatments for other parts of the body. The antegrade and retrograde catheters may align within or on either side of the occluded segment or occlusion 2300, such as cranial to (above) the occluded segment, or caudal to (below) the occluded segment, or medial and lateral, or anterior and posterior, etc.

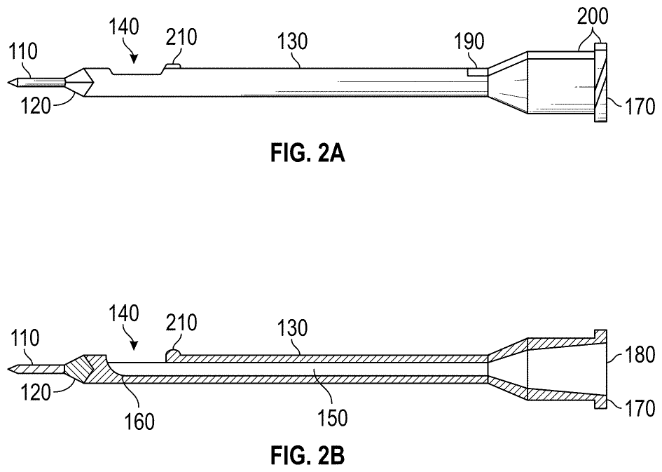

FIGS. 2A and 2B illustrate and embodiment of the needle 100. The needle 100 can be used in procedures to enable vessel 2200 access and as described herein will be used for retrograde access, though it is not limited to retrograde access and these procedures and can be used anywhere it is desirable to gain access to a vessel 2200, organ, or other region of the body.

The needle 100 of FIG. 1 illustrates a side-elevational view of a configuration and construction of a needle 100 such that it provides for improved access to and stability within smaller and/or diseased/calcified vessels 2200. The configuration of a needle 100 may have a stabilizer, such as a lance tip 110. The lance tip 110 can be smaller in diameter or cross-section than the needle shaft 130 of a needle 100. The lance tip 110 is typically small compared to the diameter of the target vessel 2200, making the vessel 2200 less likely to roll away from the lance tip 110 before it can penetrate the vessel 2200 wall, unlike a typical needle tip used in these procedures. The lance tip 110 can be symmetric, asymmetric, round, conical, contain one or more facets or surface shapes, tapers, combinations thereof, etc. The lance tip 110 diameter, or major axis/cross-section length in a non-round embodiment, may be from 10% to 90% of the needle shaft 130 diameter, more preferably from 25% to 70% of the needle shaft 130 diameter. The lance tip 110 may be closed to enhance penetration of calcified vessels 2200. The distal region of the lance tip 110 may be for example, closed, open, pointed, beveled, and/or tapered. The lance tip 110 can provide stabilization of the needle 100 within a vessel 2200, such as maintenance of position/orientation, enhanced penetration, improved accuracy of placement, especially in small diameter and/or calcified vessels 2200, etc.

The lance tip 110 and in some embodiments at least a portion of the transition section 120 may be highly echogenic to enhance ultrasound visualization and improve access success. The lance tip 110 and/or transition section 120 echogenicity may be accomplished by, for example, material selection, surface conditioning and/or surface structures (e.g. dimples, roughing the surface), internal structures or features (e.g. lattice, air gap/hollow space), echogenic coatings, and the like.

The lance tip 110 is preferably constructed such that it is not disrupted, displaced, or deforms during the procedure. This gives the user very accurate positioning and the ability to penetrate firm and/or calcified vessels 2200. The very sharp smaller diameter lance tip 110 enables improved vascular access compared to conventional needles which typically cause the vessel 2200 to roll or move away from conventional needles making access both challenging and potentially traumatic to the vessel 2200. These properties of the needle 100 also enable it to be used multiple times.

The lance tip 110 is also useful in that at least a portion of the lance tip 110 can penetrate the vessel 2200 wall opposite of the introduction site to aid in stabilizing or anchoring the needle 100 in position within the vessel 2200—often referred to as “through and through” placement. This is beneficial as it reduces or eliminates the loss of vessel engagement and repeat punctures which can result in vessel trauma, it allows for more shallow entry angles into firm and/or calcified vessels 2200—facilitating guide wire passage into the vessel 2200, and may decrease puncture site bleeding. The smaller diameter of the lance tip 110 also reduces bleeding on a through and through placement due to leaving a smaller hole in the opposite side of the vessel 2200.

The lance tip 110 may be configured in a variety of lengths depending on the target vessel lumen 2210 diameter and the intended diameter of the guide wire to be used through the needle 100. For needles 100 for use with guide wires in the 0.010″ to 0.018″ diameter range and smaller vessels 2200, 2 mm to 4 mm in diameter, the lance tip 110 may be from 0.5 mm up to 5 mm or more in length, more preferably 1.5 mm to 2.5 mm in length.

The lance tip 110 may be constructed as a separate component from the transition section 120 or may be made as a single unit and from the same material. The lance tip 110 is intended to be very stiff and the leading edge very sharp, as such the lance tip 110 can be metallic, e.g. stainless steel, tungsten, iridium, titanium, and combinations thereof), ceramic, composite, etc.

The transition section 120 of the needle 100 increases the diameter from the lance tip 110 up to the needle shaft 130. The transition section 120 typically will be positioned within the vessel lumen 2210 and allow for the needle distal lumen opening 140 to be in the vessel lumen 2210 as well. As such, the transition section 120 of needles 100 for use with guide wires in the 0.010″ to 0.018″ diameter range and smaller vessels 2200, 2 mm to 4 mm in diameter, may be from 0.5 mm up to 5 mm or more, more preferably 1.0 mm to 1.5 mm in length.

The transition section 120 may be configured to include facets on the surface. In other embodiments, the transition section 110 may be symmetric, asymmetric, conical, contain one or more facets or surface shapes, as long as it transitions to the needle shaft 130. The transition section 120 can be made of similar materials as the lance tip, previously described, as the needle shaft 130, or of other materials.

The needle shaft 130 can be considered the main body of the needle 100, with a needle distal lumen opening 140, and a needle lumen 150 and needle ramp 160 as illustrated in FIG. 2B. The needle lumen 150 extends from the needle hub proximal lumen opening 180, through the needle shaft 130, and to the needle distal lumen opening 140. The needle lumen 150 contains a needle ramp 160 which is a way to direct a guide wire from travelling substantially longitudinal (parallel) to the length of the needle shaft 130 to an angle with respect to the longitudinal axis of the needle shaft 130 as it exits the needle distal lumen opening 140. The needle ramp 160 may be a flat, radiused/curved shape, any configuration that changes the angle away from the longitudinal axis of the needle shaft 130 as a guide wire exits the needle distal lumen opening 140. The needle ramp 160 is configured to deflect the guide wire out of the needle shaft 130 at an angle within the range of from about 10 degrees to about 60 degrees or more from the longitudinal axis.

The needle shaft 130 is sized based on the intended diameter of the guide wire to be used through the needle 100. For needles 100 for use with guide wires in the 0.010″ to 0.018″ diameter range, the needle lumen 150 is 0.001″ to 0.020″ larger in diameter than the intended guide wire diameter, more preferably 0.002″ to 0.008″ larger in diameter. The needle shaft 130 wall thickness is generally 0.003″ to 0.010″, preferably 0.003″ to 0.006″. For example, a needle 100 for use with a 0.014″ guide wire would have a needle lumen 150 of 0.017″ to 0.018″ with a wall thickness of 0.005″ for an outside diameter of 0.027″. Needle shaft 130 length is 3 cm to 10 cm, more preferably 4 cm to 7 cm in length. The needle shaft 130 may be made of similar materials as the lance tip 110 and/or transition section 120 previously described. For example, the lance tip 110 and transition section 120 may be tungsten while the needle shaft 130 may be stainless steel, such as a 300 or 400 series. In another example, a needle 100 for use with a 0.014″ diameter guide wire, the needle lumen 150 is 0.018″ in diameter, the needle shaft 130 wall thickness is 0.006″ for an outside diameter of 0.030″. Needle shaft 130 length is 6 cm and made of stainless steel. Lance tip 110 is 2 mm in length and the transition section 120 is 1 mm in length, also made of stainless steel. Distance from the distal end of the needle 100 to the distal side of the needle distal lumen opening 140 is 3 mm.

In another embodiment the needle distal lumen opening 140 may be in part or entirely in the transition section 120 which places the needle ramp 160 also in part or entirely in the transition section 120.

Markers may be incorporated to provide for an indicium of rotational orientation of the needle distal lumen opening 140. A needle shaft marker 190 may be placed on or made as part of the needle shaft 130 in the same rotational position as the needle distal lumen opening 140. The needle shaft marker 190 may be visual, such as a different color (e.g. using ink or paint) and/or it may be a different texture or have different reflectivity than the needle shaft 130. The needle shaft marker 190 may also be or instead of visual may be tactile, such as a raised section or sufficiently different surface characteristics as to be felt through a gloved hand.

On the proximal region of the needle 100 there is a needle hub 170. The needle hub 170 serves as an entry point for introducing a guide wire into the needle 100 through the needle hub proximal lumen opening 180. The needle hub 170 consists of a Luer fitting or tapered section which may have one or more wings extending from the needle hub 170 and/or needle shaft 130. The needle hub 170 may be attached to the needle shaft 130 such as by bonding, ultrasonic welding, or may be molded onto the needle shaft 130. The needle hub 170 is typically plastic, such as polyethylene, polyurethane, polycarbonate, etc. The needle hub 170 may also be metallic, for example, 300 or 400 series stainless steel.

The needle hub 170 may have a needle hub marker 200 be placed on or made as part of the needle hub 170 in the same rotational position as the needle distal lumen opening 140. The needle hub marker 200 may be visual, such as a different color (e.g. ink or paint) and/or it may be a different texture or have different reflectivity than the needle hub 170. The needle hub marker 200 may also be or instead of visual may be tactile, such as a raised section or sufficiently different surface characteristics as to be felt through a gloved hand.

The needle shaft 130 may contain a bumper 210, wherein engagement of the bumper 210 at the surface of the vessel 2200 can assist the user in aligning the needle distal lumen opening 140 within the vessel 2200. This is accomplished by tactile and/or visual feedback. The bumper 210 also serves as a rotational orientation marker. The bumper 210 may be of a different radiopacity than the needle shaft 130.

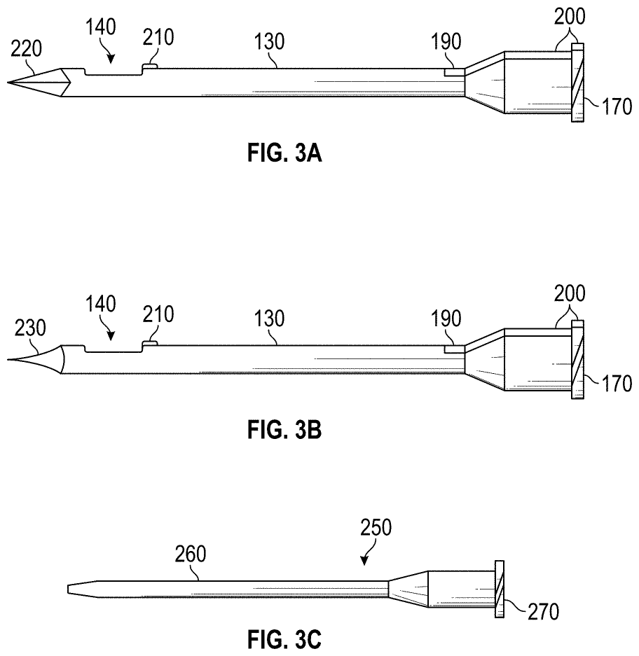

In another embodiment illustrated in FIG. 3A-B, the lance tip 110 and transition section 120 are effectively a single element tip 220, or a combination tip 230. In these embodiments, the single element tip 220 or combination tip 220 serves to both penetrate a potentially diseased/calcified/mobile vessel 2200 as well as stabilize the needle 100 in the vessel 2200. The single element tip 220 or combination tip 230 can be symmetric, asymmetric, conical, contain one or more facets or surface shapes, tapers, combinations thereof, etc.

In another embodiment the needle shaft 130, transition section 120, lance tip 110, single element tip 220, or combination tip 230, or any combination thereof may have a curve or bend to facilitate vessel 2200 access and positioning.

In an embodiment, a microcatheter 250 may be included for use with the needle 100 or retrograde guide wire 310 or other guide wires as shown in FIG. 3C. The microcatheter 250 may be introduced over a guide wire to enlarge the passageway through/around/past an occlusion 2300 prior to introduction of a retrograde catheter. The microcatheter 250 has a microcatheter shaft 260 with a through lumen and a thin wall to pass into the vessel 2200, typically over a guide wire. The microcatheter 250 can be used to provide vascular access for passing the retrograde guide wire 310 or other guide wires into the target vessel 2200. The microcatheter shaft 260 can be constructed of one or more polymers, composites, metals, or combinations thereof. Examples include polyethylene, polyurethane, nylon, Pebax®, polyimide, fluoropolymers, carbon fiber, stainless steel, nitinol, titanium, etc. The microcatheter shaft 260 can be constructed to be radiopaque by adding a radiopaque material in a polymer construction, such as barium sulfate or tantalum or a radiopaque braid/coil, or by forming it from a metal or a coated metal (e.g. gold-coated stainless steel), or a combination thereof. Typical size of the microcatheter shaft 260 for a 0.014″ retrograde guide wire 310 is an inside diameter of 0.0145″ to 0.0200″, with a tip region typically 0.0140″ to 0.0155″. The wall thickness of the microcatheter shaft 260 is typically 0.002″ to 0.010″, more preferably 0.002″ to 0.005″. The length of the microcatheter shaft 260 is typically 2.5 cm to 10 cm, more preferably 3.5 cm to 6.5 cm. The microcatheter hub 270 consists of a Luer fitting or tapered section which may have one or more wings extending from the microcatheter hub 270 and/or microcatheter shaft 260. The microcatheter hub 270 may be attached to the microcatheter shaft 260 such as by bonding, ultrasonic welding, or may be molded onto the microcatheter shaft 260. The microcatheter hub 270 is typically plastic, such as polyethylene, polyurethane, polycarbonate, etc. The microcatheter hub 270 may also be metallic, for example, 300 or 400 series stainless steel.

Having established retrograde access with a guide wire in place, a retrograde catheter may now be introduced into the vessel 2200. The retrograde catheter is particularly designed to traverse an occlusion 2300, such as a chronic total occlusion (CTO), typically by creating a dissection plane. It will be understood that though the retrograde catheter and methods are described primarily with reference to lower extremity vascular procedures creating a directional dissection plane in the vessel 2200 wall, such as between the intima and media, through the occlusion 2300 itself, within the vessel lumen 2210, or a combination thereof, the retrograde catheter can also be used in treatments for other parts of the body.

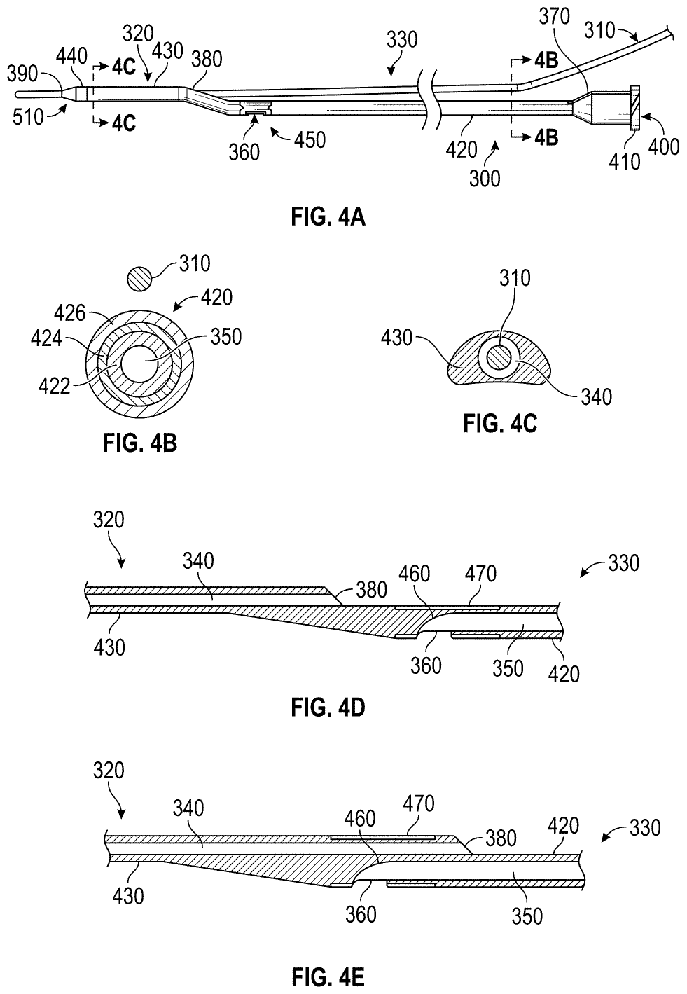

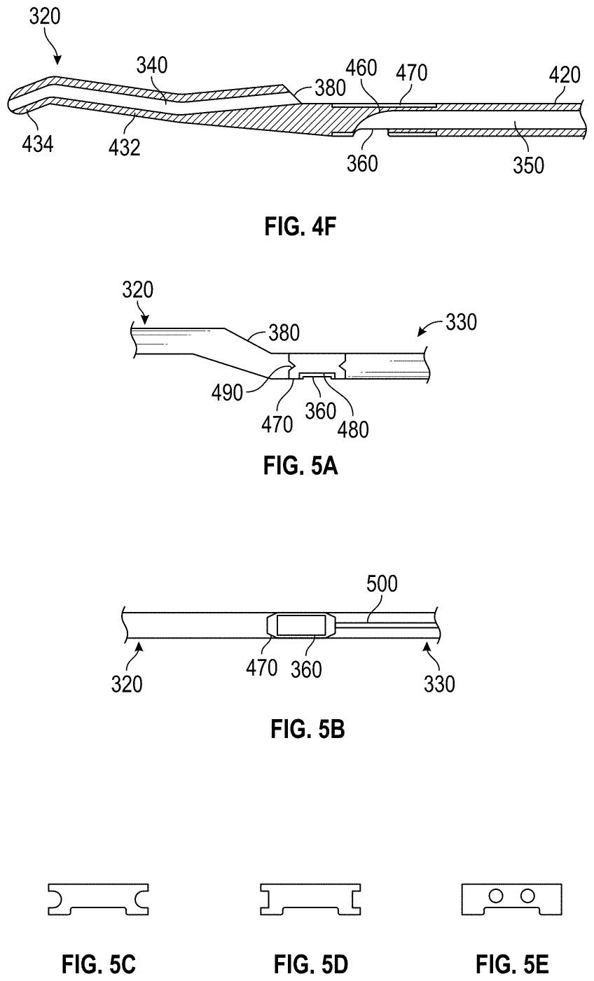

The retrograde catheter will be described sized for use in the lower extremities over a 0.014″ guide wire, though other sizes, lengths, and diameters of the retrograde catheter and guide wire are within the scope of the invention. As illustrated in FIG. 4A, the retrograde catheter may be designed in a mini-rail configuration—a mini-rail retrograde catheter 300, wherein only a portion of the mini-rail retrograde catheter 300 contains or rides over the retrograde guide wire 310, and the remainder of the mini-rail retrograde catheter 300 does not contain the retrograde guide wire 310.

The mini-rail retrograde catheter 300 contains two lumens, a mini-rail guide wire lumen 340 and a rendezvous guide wire lumen 350. The mini-rail guide wire lumen 340 has a mini-rail lumen proximal opening 380 or port and a mini-rail lumen distal opening 390. The rendezvous guide wire lumen 350 has a proximal opening 400 or port adjacent the mini-rail retrograde catheter hub 410 and a side port/exit port 360.

The mini-rail retrograde catheter hub 410 serves as an entry point for introducing a rendezvous guide wire 2000 into the mini-rail retrograde catheter 300 through the proximal opening 400. The mini-rail retrograde catheter hub 410 consists of a Luer fitting or tapered section. The mini-rail retrograde catheter hub 410 may be attached to the rendezvous guide wire shaft 420 such as by bonding or ultrasonic welding, or may be molded onto the rendezvous guide wire shaft 420. The mini-rail retrograde catheter hub 410 is typically plastic, such as polyethylene, polyurethane, polycarbonate, etc., or may be metallic or composite or a combination thereof.

The rendezvous guide wire shaft 420 may be single lumen throughout the majority of its length and constructed to provide for suitable pushability (longitudinal motion) and torquability (rotational motion) such that the mini-rail retrograde catheter 300 can be steered, rotated, and advanced within a vessel 2200 as well as enable blunt or cutting dissection through a vessel 2200 wall or through an occlusion 2300 or both. The rendezvous guide wire shaft 420 is typically round in cross-section, see FIG. 4B, and can be constructed of one or more polymers, composites, metals, or combinations thereof. Examples include polyethylene, polyurethane, nylon, Pebax, polyimide, fluoropolymers, carbon fiber, stainless steel, nitinol, titanium, etc. The rendezvous guide wire shaft 420 can be made at least in part as a laminate of various materials, such as a liner 422, a reinforcement 424 (e.g. braid, coil), and a jacket 246. The rendezvous guide wire shaft 420 as constructed with allow for lateral motion control as well. The rendezvous guide wire shaft 420 may be constructed to be at least in part radiopaque by adding a radiopaque material in a polymer construction, such as barium sulfate or tantalum or a radiopaque reinforcement 424 braid/coil, or by forming it from a metal or a coated metal (e.g. gold-coated stainless steel), or a combination thereof. Also, the rendezvous guide wire shaft 420 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the mini-rail rendezvous catheter 300 using fluoroscopy and/or ultrasound to better improve positioning and orientation.

The rendezvous guide wire shaft 420, as well as any part of a retrograde catheter may include one or more coils, braiding, materials, etc., to enhance the properties. For example, a reinforcement 424 (e.g. braid, coil) section within the rendezvous guide wire shaft 420 would enhance torquability and a coil within the rendezvous guide wire shaft 420 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

The rendezvous guide wire shaft 420 and/or mini-rail retrograde catheter hub 410 may have a visual and/or tactile indicators as an indicium of the rotational orientation of the exit port 360 and/or mini-rail proximal lumen opening 380, or other more distal mini-rail retrograde catheter 300 element. For example, the rendezvous guide wire shaft 420 may have a stripe 500 and the mini-rail retrograde catheter hub 410 may have a stripe 500 and/or hub marker 370 as a visual and/or tactile indicator of the rotational orientation of the exit port 360. The stripe 500 and/or hub marker 370 may be colored differently than the rendezvous guide wire shaft 420 or mini-rail retrograde hub 410 to provide visual feedback and raised and/or textured to provide tactile feedback. Visual and tactile markers such as these may be employed on all versions of the catheters of the invention.

The distal region 320 of the mini-rail retrograde catheter 300 contains at least a portion of the mini-rail shaft 430. A portion or the entire length of the mini-rail shaft 430 is configured to enable dissection through a vessel 2200 wall, such as subintimal, intimal, intramural (medial), subadventitial, through the occlusion 2300 itself, within the vessel lumen 2210, or a combination thereof. To facilitate dissection and/or manipulation, the distal region 320 may be bent or curved or have multiple bends or curves in one or more directions. FIG. 4F illustrates one example of a distal region 320 with multiple curves. Illustrated is a first mini-rail shaft region 432, in which the distal region 320 has a bend or curve with respect to the longitudinal axis of the rendezvous guide wire shaft 420 as well as a second mini-rail shaft region 434, in which the distal region 320 has a second bend or curve with respect to the longitudinal axis of the first mini-rail shaft region 432. This one or more bend or curve configuration may be used on any of the retrograde and antegrade catheters of the invention. To facilitate orientation within the vessel 2200, alignment with an antegrade catheter, and passage across occluded segments allowing dissection similar to surgical elevators (as such, a remotely introduced endovascular surgical tool), the mini-rail shaft 430 may be rounded or non-round shaped such as a flattened, spatulated, crescent shape, edgy, etc. A cross-section of the mini-rail shaft 430 with a spatulated shape is illustrated in FIG. 4C. The distal tip and/or some or all of the length of the mini-rail shaft 430 may be comparatively very hard and/or resistant to compression so as to not collapse on the retrograde guide wire 310 or longitudinally deform, compress, or buckle when being advanced through tissue or an occlusion 2300. The distal tip or adjacent region of the mini-rail shaft 430 may contain a tip dissection feature 510 which may be blunt, facilitating blunt dissection, or cutting or edgy to create the dissection plane. The tip dissection feature 510 may be the shape of the feature, such as a radiused surface or a sharp edged tip in one or more planes, examples being 1) a radius in the plane of the exit port 360 and a sharp edge perpendicular to that, similar to a radiused wedge with a sharp edge, or 2) a radius in both of these planes forming a more blunt tip dissection feature 510. The tip dissection feature 510 may be formed in the distal region 320 and/or added to the distal region 320 as a separate component, such as a metallic or polymer element, or made as part of the retrograde tip radiopaque marker 440, which may extend out to the distal tip. The distal tip or adjacent region of the mini-rail shaft 430 may contain a retrograde tip radiopaque marker 440, made from metals such as gold, platinum, iridium, tantalum, or combinations thereof and be shaped to include the tip dissection feature 510. The distal tip or adjacent region of the mini-rail shaft 430 may be made from a polymer containing a radiopaque substance, such as barium sulfate, or the entire mini-rail shaft 430 may be radiopaque and/or echogenic.

The mini-rail shaft 430 may be single lumen throughout the majority of its length and constructed to provide for pushability (longitudinal motion) and torquability (rotational motion) such that the mini-rail retrograde catheter 300 can be steered, rotated, and advanced within a vessel 2200 as well as enable dissection through a vessel 2200 wall or through an occlusion 2300 or both. The mini-rail shaft 430 can be constructed of one or more polymers, but may include composites, metals, or combinations thereof. Examples include polyethylene, polyurethane, nylon, Pebax, polyimide, fluoropolymers, etc. The mini-rail shaft 430, as well as any part of a retrograde catheter may include a reinforcement 424, one or more coils, braiding, laminated sections, etc., to enhance the properties as previously described for the rendezvous guide wire shaft 420. Additionally, the mini-rail shaft 430 or a portion thereof may be constructed to enhance visualization through radiopacity and/or echogenicity as previously described for the rendezvous guide wire shaft 420.

The mini-rail retrograde catheter 300 contains a ramp section 450, which can be seen in further detail in FIGS. 4D-E. A ramp 460 is preferably located near the distal end region of the rendezvous guidewire lumen 350 and is used to direct the rendezvous guide wire 2000 from travelling substantially longitudinal (parallel) to the length of the rendezvous guide wire shaft 420 to an angle with respect to the longitudinal axis of the rendezvous guide wire shaft 420 as it exits the exit port 360. The angle the rendezvous guide wire 2000 exits the exit port 360 can influence the ability to penetrate any tissue (e.g. intima 2220) and enter a side port/window 990, as does the shape of the tip of the rendezvous guide wire 2000. A preferable angle is 5 degrees to 80 degrees off axis, more preferably an angle of 20 degrees to 60 degrees. The ramp 460 may be a flat, radiused/curved shape, any configuration that changes the angle away from the longitudinal axis of the rendezvous guide wire shaft 420 as the rendezvous guide wire 2000 exits the exit port 360.

The ramp section 450 may serve as a transition from the distal region 320 to the proximal region 330. The mini-rail lumen proximal opening 380 may be located relatively distal to the exit port 360, as illustrated in FIG. 4D, the mini-rail lumen proximal opening 380 may be located relatively proximal to the exit port 360 as illustrated in FIG. 4E, or the mini-rail lumen proximal opening 380 may substantially coincide with the exit port 360.

The ramp section 450 may have a different radiopacity or echogenicity than other portions of the mini-rail retrograde catheter 300. It is preferable to visualize the longitudinal and lateral position of the exit port 360 as well as rotational orientation of the exit port 360. FIG. 5A illustrates one embodiment of an exit port radiopaque marker 470 that provides for longitudinal, lateral, and rotational identification of the exit port 360. When viewed in the orientation in FIG. 5A, the exit port 360 can be seen as a notch 480 in the exit port radiopaque marker 470, as well as the chevrons 490 can be visualized. Looking at FIG. 5B, when rotated 90 degrees, neither the notch 480 is visible as such nor are the full chevrons 490.

Other embodiments of the exit port radiopaque marker 470 can be seen in FIGS. 5C-E with rotational alignment features, or simply a notch 480 without any other additional features.

In other embodiments of the exit port radiopaque marker 470, the exit port radiopaque marker 470 can be made of two or more individual radiopaque markers. Both radiopaque markers can be used as described above or one radiopaque marker can be used for identifying orientation in one rotational alignment and the other radiopaque marker for identifying rotational alignment in a different orientation.

The ramp section 450 can be reinforced, such as with a metal, fiber, or polymer element, braid; coil, safety wire/strap/cable, etc., or made as a separate component and/or integrated into the mini-rail retrograde catheter 300. This is to provide an additional level of safety that the distal region 320 and proximal region 330 never separate. This can also provide for reducing the propensity for the mini-rail retrograde catheter 300 to deform, buckle, or kink in this region. The ramp section 450 reinforcement may be constructed separately or as part of the exit port radiopaque marker 470 or other longitudinal and/or lateral and/or rotational marker (radiopaque and/or echogenic).

As described for use over a 0.014″ diameter retrograde guide wire, the mini-rail retrograde catheter 300 is typically within the range of from about 65 cm to about 135 cm in working length—from the distal tip to the distal end region of the mini-rail retrograde catheter hub 410, and for example may be about 65 cm, 80 cm, or about 135 cm depending upon the vascular access point, the location of the occlusion 2300, and intended clinical performance. The mini-rail guide wire lumen 340 is nominally 0.017″ in diameter and may be smaller at the distal tip, e.g. 0.015″, so as to have a tighter fit to the guide wire facilitating dissection/occlusion crossing/penetration without allowing tissue or occlusive material into the annular area between the guide wire and the mini-rail shaft 430, however, the mini-rail guide wire lumen 340 can be constructed for any guide wire diameter. The length of the mini-rail guide wire lumen 340 can be from 1 cm to 10 cm or more. More preferably, the length of the mini-rail guide wire lumen is from 1 cm to 3 cm. The short axis (if not round) wall thickness of the mini-rail shaft 430 is from 0.0015″ to 0.0200″ and may taper or blend to an edge. More preferably, the short axis wall thickness of the mini-rail shaft 430 is from 0.003″ to 0.0100″ and may taper or blend to an edge. This results in a distal section short axis profile 3.0 Fr or less, or more preferably 2.6 Fr or less. The rendezvous guide wire lumen 350 would typically be configured for a 0.010″ to 0.018″ rendezvous guide wire 2000, preferably for a 0.014″ rendezvous guide wire 2000.

Portions or all of the mini-rail retrograde catheter 300 may be coated to enhance reflectivity, increase lubricity, increase stiffness, etc. Examples of coatings include a lubricious coating such as silicone or fluoropolymer, hydrophilic, hydrophobic, etc., on the outside of the mini-rail shaft 430 and/or ramp section 450 and/or tip dissection feature 510 to reduce the force needed to achieve dissection/occlusion crossing/penetration. A polymer surface coating can be applied to the ramp section to further increase the reflectivity of that area of the mini-rail retrograde catheter 300. An internal or external coating (or laminate/layer) of polyimide used with a Pebax would increase stiffness and pushability of the rendezvous guide wire shaft 420. Coatings may be used within the lumens, on the outside, or anywhere as part of the catheters of the present invention to achieve the desired effect(s).

An example construction of an 80 cm overall length mini-rail retrograde catheter 300 for use with a 0.014″ rendezvous guide wire 2000 is as follows. A mini-rail guide wire lumen 340 of 0.017″ proximally that tapers down to 0.015″ distally. A mini-rail guide wire lumen 340 of 2 cm in length with a mini-rail shaft 430 having a spatulated, slightly concave, cross-section with a short axis wall thickness of 0.005″ for a short axis profile of 0.025″ to 0.027″ (1.9 Fr to 2.1 Fr), and a long axis wall thickness of 0.0085″ for a profile of 0.034″ (2.6 Fr). The mini-rail shaft 430 comprised of Pebax 72D loaded with 40% BaSO₄ for radiopacity. A retrograde tip radiopaque marker 1 mm in length comprised of 90% Pt 10% Ir with a wall thickness of 0.002″, located 1 mm proximal of the mini-rail lumen distal opening 390. A tip dissection feature 510 being bullet shaped when viewed perpendicular to the long axis section and edgy when viewed perpendicular to the short axis section. The mini-rail shaft 430 having a 10-degree angulation in the opposite direction of the exit port 360, effectively being a 10-degree angulation from the longitudinal axis of the rendezvous guide wire shaft 420.

The rendezvous guide wire shaft 420 having a rendezvous guide wire lumen 350 inside diameter of 0.017″ throughout its length. The rendezvous guide wire shaft 420 having a round cross-section constructed with an inner liner 422 of Pebax 35D to 72D (more preferably 55D), a 304V stainless steel braid 424 over the inner liner 422 of Pebax 55D with 8 to 32 wires (more preferably 16 wires), 0.001″ to 0.003″ diameter round or flat (typically 0.0005″ to 0.0020″ thick by 0.001″ to 0.005″ wide wires) (more preferably 0.002″ round), typically 30 to 50 pics per inch (more preferably 40 pics per inch), with an outer jacket 426 of Pebax 55 to 72D (more preferably 72D). The overall rendezvous guide wire shaft 420 wall thickness being 0.0085″ for an outside diameter of 0.034″ (2.6 Fr).

The ramp section 450 being Pebax 72D with reinforcement 424 from the rendezvous guide wire shaft 420 braid extending into the ramp section 450. The location of the mini-rail lumen proximal opening 380 being 3 mm proximal of the exit port 360, similar to that shown in FIGS. 4A and 4D. The exit port 360 having a length of 0.030″ and a width of 0.017″. An exit port radiopaque marker 470 as shown in FIGS. 5A-5B, made of 90% Pt 10% Ir with a wall thickness of 0.002″, and overall length of 0.130″ with a notch 480 length of 0.050″.

A mini-rail retrograde catheter hub 410 adjacent the proximal end of the rendezvous guide wire shaft 420. The mini-rail retrograde catheter hub 410 being a Luer fitting with an internal taper to form a smooth transition from the internal surface of the Luer fitting to the rendezvous guide wire lumen 350. A visual hub marker 370 on the mini-rail retrograde catheter hub 410 aligned with the exit port 360 and a stripe 500 on the rendezvous guide wire shaft 420 continuing from the hub marker 370 running to and aligned with the exit port 360.

The distal 15 cm of the mini-rail retrograde catheter 300 having an external hydrophilic coating for improved lubricity.

Another example of a mini-rail retrograde catheter 300 with a longer mini-rail guide wire lumen 340 than in the previous example is described. In this example, the mini-rail guide wire lumen 340 is 10 cm to 15 cm in length, more preferably 12 cm in length. Materials are similar to the above example. The ramp section 450 is similarly reinforced with the location of the mini-rail lumen proximal opening 380 being 3 cm proximal of the mini-rail distal lumen opening 390, similar to that shown in FIG. 4E. This example provides for a longer length of catheter on the guide wire which may provide for additional pushability through tissue, occlusion, etc.

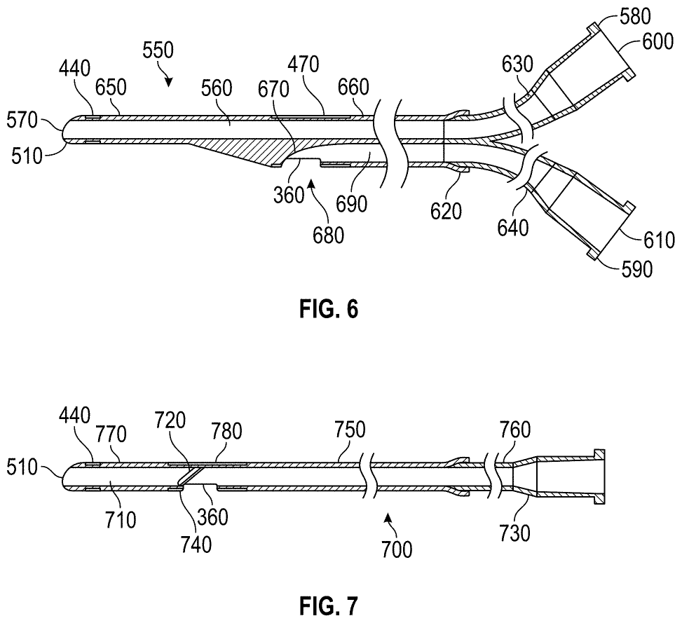

Another embodiment of a retrograde catheter is an over the wire configuration as illustrated in FIG. 6. The over the wire retrograde catheter 550 in FIG. 6 contains two lumens, an OTWR guide wire lumen 560 and an OTWR rendezvous guide wire lumen 690. The OTWR guide wire lumen 560 has an OTWR distal opening 570 and proximally terminates at the OTWR guide wire hub proximal opening 600 or port. The OTWR rendezvous guide wire lumen 690 extends from the exit port 360 to the OTWR rendezvous hub proximal opening 610 or port. The OTWR adapter interface 620 is where the over the wire retrograde catheter 550 multilumen shaft region terminates and/or enters a traditional multilumen catheter Y-arm, or splits into pig tails, or suitable adaptor(s), such as the OTWR guide wire pigtail 630 and OTWR rendezvous pigtail 640.

The OTWR guide wire hub 580 consists of a Luer fitting or tapered section. The OTWR guide wire hub 580 may be attached to the OTWR guide wire pigtail 630 such as by bonding, ultrasonic welding, or may be molded onto the OTWR guide wire pigtail 630. The OTWR guide wire hub 580 is typically plastic, such as polyethylene, polyurethane, polycarbonate, etc., or may be metallic or composite or a combination thereof. The OTWR rendezvous hub 590 consists of a Luer fitting or tapered section. The antegrade OTWR rendezvous hub 590 may be attached to the OTWR rendezvous pigtail 640 such as by bonding, ultrasonic welding, or may be molded onto the OTWR rendezvous pigtail 640. The OTWR rendezvous hub 590 is typically plastic, such as polyethylene, polyurethane, polycarbonate, etc., or may be metallic or composite or a combination thereof. The pigtails are typically polymer-based and may be laminated, contain braids, or one or more coils.

The over the wire retrograde catheter 550 contains an exit port 360. A OTWR ramp 670 is located near the distal end region of the rendezvous guidewire lumen 350 and is used to direct the rendezvous guide wire 2000 from travelling substantially longitudinal (parallel) to the length of the OTWR multilumen shaft 660 to an angle with respect to the longitudinal axis of the OTWR multilumen shaft 660 as it exits the exit port 360. The angle the rendezvous guide wire 2000 exits the exit port 360 can influence the ability to penetrate any tissue (e.g. intima 2220) and enter a window 990, as does the shape of the tip of the rendezvous guide wire 2000. A preferable angle is 5 degrees to 80 degrees off axis, more preferably an angle of 20 degrees to 60 degrees. The OTWR ramp 670 may be a flat, radiused/curved shape, any configuration that changes the angle away from the longitudinal axis of the OTWR multilumen shaft 660 as the rendezvous guide wire 2000 exits the exit port 360.

The OTWR multilumen shaft 660 is constructed to provide for suitable pushability (longitudinal motion) and torquability (rotational motion) such that the over the wire retrograde catheter 550 can be steered, rotated, and advanced within a vessel 2200 as well as enable blunt or cutting dissection through a vessel 2200 wall or through an occlusion 2300 or both. The OTWR multilumen shaft 660 is typically round in cross-section and can be constructed of one or more polymers, composites, metals, or combinations thereof. Examples include polyethylene, polyurethane, nylon, Pebax, polyimide, fluoropolymers, carbon fiber, stainless steel, nitinol, titanium, etc. The OTWR multilumen shaft 660 can be made at least in part as a laminate of various materials. The OTWR multilumen shaft 660 as constructed with allow for lateral motion control as well. The OTWR multilumen shaft 660 may be constructed to be radiopaque by adding a radiopaque material in a polymer construction, such as barium sulfate or tantalum or a radiopaque reinforcement 424 (e.g. braid, coil), or by forming it from a metal or a coated metal (e.g. gold-coated stainless steel), or a combination thereof. Also, the OTWR multilumen shaft 660 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the over the wire retrograde catheter 550 using fluoroscopy and ultrasound to better improve positioning and orientation.

The OTWR multilumen shaft 660, as well as any part of a retrograde catheter may include one or more coils, braiding, etc., to enhance the properties. For example, a reinforcement 424 (e.g. braid, coil), section within the OTWR multilumen shaft 660 would enhance torquability and a coil within the OTWR multilumen shaft 660 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

A previously illustrated in FIG. 4A, similar visual and/or tactile indicators can be used on the OTWR multilumen shaft 660 and/or OTWR rendezvous hub 590 for indicating the rotational orientation of the exit port 360 or other more distal over the wire retrograde catheter 550 element. For example, OTWR rendezvous hub 590 and/or the OTWR multilumen shaft 660 may have a visual indicator of the rotational orientation of the exit port 360. The OTWR multilumen shaft 660 may have a stripe 500 as a visual indicator to provide visual feedback and/or raised and/or textured to provide tactile feedback of the exit port 360.

The OTWR single lumen shaft 650 is constructed to provide for pushability (longitudinal motion) and torquability (rotational motion) such that the over the wire retrograde catheter 550 can be steered, rotated, and advanced within a vessel 2200 as well as enable dissection through a vessel 2200 wall or through an occlusion 2300 or both. The OTWR single lumen shaft 650 can be constructed of one or more polymers, but may include composites, metals, or combinations thereof. Examples include polyethylene, polyurethane, nylon, Pebax, polyimide, fluoropolymers, etc. The OTWR single lumen shaft 650, as well as any part of a retrograde catheter may include a reinforcement 424, one or more coils, braiding, laminated sections, etc., to enhance the properties as previously described for the OTWR multilumen shaft 660. Additionally, the OTWR single lumen shaft 650 may be constructed to enhance visualization through radiopacity and/or echogenicity as previously described for the OTWR multilumen shaft 660.

At least a portion of the OTWR single lumen shaft 650 and/or tip dissection feature 510 and optionally at a portion of the OTWR multilumen shaft 660 is configured to enable dissection through a vessel 2200 wall, such as subintimal, intimal, intramural (medial), subadventitial, through the occlusion 2300 itself, within the vessel lumen 2210, or a combination thereof. To facilitate orientation within the vessel 2200, alignment with an antegrade catheter, and dissection, a portion of the OTWR single lumen shaft 650 may be round or non-round shaped such as a flattened, spatulated, crescent shape, edgy, etc. The distal tip and/or some or all of the length of the OTWR single lumen shaft 650 may be comparatively very hard so as to not collapse on the retrograde guide wire 310 or longitudinally deform, compress, or buckle when being advanced through tissue or an occlusion 2300. The distal tip or adjacent region of the OTWR single lumen shaft 650 may contain a tip dissection feature 510 which may be blunt, facilitating blunt dissection, or cutting or edgy to create the dissection plane. The tip dissection feature 510 may be the shape of the feature, such as a radiused surface or a sharp edged tip in one or more planes, examples being 1) a radius in the plane of the exit port 360 and a shape edge perpendicular to that, similar to a radiused wedge with a sharp edge, or 2) a radius in both of these planes forming a more blunt tip dissection feature 510. The tip dissection feature 510 may be formed in the OTWR single lumen shaft 650 and/or added to the OTWR single lumen shaft 650 as a separate component, such as a metallic or polymer element, or made as part of a tip radiopaque marker, which may extend out to the distal tip. The distal tip or adjacent region of the OTWR single lumen shaft 650 may contain a tip radiopaque marker, made from metals such as gold, platinum, iridium, tantalum, or combinations thereof and be shaped to include the tip dissection feature 510. The distal tip or adjacent region of the OTWR single lumen shaft 650 may be made from a polymer containing a radiopaque substance, such as barium sulfate, or the entire OTWR single lumen shaft 650 may be radiopaque and/or echogenic.

The over the wire retrograde catheter 550 contains an OTWR ramp section 680. The OTWR ramp section 680 is located near the distal end region of the OTWR rendezvous guidewire lumen 690 and is used to direct the rendezvous guide wire 2000 from travelling substantially longitudinal (parallel) to the length of the OTWR multilumen shaft 660 to an angle with respect to the longitudinal axis of the OTWR multilumen shaft 660 as it exits the exit port 360. The angle the rendezvous guide wire 2000 exits the exit port 360 can influence the ability to penetrate any tissue (e.g. intima 2220) and enter a window 990, as does the shape of the tip of the rendezvous guide wire 2000. A preferable angle is 5 degrees to 80 degrees off axis, more preferably an angle of 20 degrees to 60 degrees. The OTWR ramp 670 may be a flat, radiused/curved shape, any configuration that changes the angle away from the longitudinal axis of the OTWR multilumen shaft 660 as the rendezvous guide wire 2000 exits the exit port 360.

The OTWR ramp section 680 may have a different radiopacity or echogenicity than other portions of the over the wire retrograde catheter 550. It is preferable to visualize the longitudinal and lateral position of the exit port 360 as well as rotational orientation of the exit port 360. FIG. 5A illustrates one embodiment of an exit port radiopaque marker 470 that provides for longitudinal, lateral, and rotational identification of the exit port 360 that may be employed on the over the wire retrograde catheter 550. When viewed in the orientation in FIG. 5A, the exit port 360 can be seen as a notch 480 in the exit port radiopaque marker 470, as well as the chevrons 490 can be visualized. Looking at FIG. 5B, when rotated 90 degrees, neither the notch 480 is visible as such nor are the full chevrons 490.

Other embodiments of the exit port radiopaque marker 470 that may be employed on the over the wire retrograde catheter 550 can be seen in FIGS. 5C-E with rotational alignment features, or simply a notch 480 without any other additional features.

In other embodiments of the exit port radiopaque marker 470, the exit port radiopaque marker 470 can be made of two or more individual radiopaque markers. Both radiopaque markers can be used as described above or one radiopaque marker can be used for identifying orientation in one rotational alignment and the other radiopaque marker for identifying rotational alignment in a different orientation.

The OTWR ramp section 680 can contain a reinforcement 424, such as with a metal, fiber, or polymer element; braid; coil; safety wire/strap/cable, etc., or made as a separate component and/or integrated into the over the wire retrograde catheter 550. This is to provide an additional level of safety that the distal region 320 and proximal region 330 never separate. This can also provide for reducing the propensity for the mini-rail retrograde catheter 300 to deform, buckle, or kink in this region. The ramp section 450 reinforcement may be constructed separately or as part of the exit port radiopaque marker 470 or other longitudinal and/or lateral and/or rotational marker (radiopaque and/or echogenic).

As described for use with a 0.014″ diameter retrograde guide wire 310 and a 0.014″ rendezvous guide wire 2000, the over the wire retrograde catheter 550 is typically within the range of from about 65 cm to about 135 cm in working length—from the distal tip to the OTWR adapter interface 620, and for example may be about 65 cm, 80 cm, or about 135 cm depending upon the vascular access point, the location of the occlusion 2300, and intended clinical performance. The length of the OTWR single lumen shaft 650 can be from 1 cm to 10 cm or more. More preferably, the length of the OTWR single lumen shaft 650 is from 1 cm to 3 cm. The OTWR multilumen shaft 660 diameter would typically be from 0.043″ to 0.072″ in diameter, more preferably from 0.043″ to 0.053″ in diameter. The OTWR single lumen shaft 650 is nominally 0.017″ in inside diameter and may be smaller at the distal tip, e.g. 0.015″, so as to have a tighter fit to the guide wire facilitating dissection/occlusion crossing/penetration without allowing tissue or occlusive material into the annular area between the guide wire and the OTWR single lumen shaft 650. The short axis (if not round) wall thickness of the OTWR single lumen shaft 650 is typically from 0.0015″ to 0.0200″ and may taper or blend to an edge. More preferably, the short axis wall thickness of the OTWR single lumen shaft 650 is from 0.003″ to 0.0100″ and may taper or blend to an edge. This results in an OTWR single lumen shaft 650 short axis profile 3.0 Fr or less, or more preferably 2.6 Fr or less.

Portions or all of the over the wire retrograde catheter 550 may be coated to enhance reflectivity, increase lubricity, increase stiffness, etc. Examples of coatings include a lubricious coating such as silicone or fluoropolymer, hydrophilic, hydrophobic, etc., on the outside on the outside of the OTWR single lumen shaft 650 and OTWR ramp section 680 to reduce the force needed to achieve dissection/occlusion crossing/penetration. A polymer surface coating can be applied to the OTWR ramp section 680 to further increase the reflectivity that area of the over the wire retrograde catheter 550. An internal or external coating (or laminate/layer) of polyimide with a Pebax would increase stiffness and pushability of the OTWR multilumen shaft 660 and/or OTWR single lumen shaft 650.

FIG. 7 illustrates an embodiment of an over the wire configuration which makes use of a moveable ramp 720 to allow for a single lumen very low-profile design. Having a moveable ramp 720 enables a single lumen over the wire configuration wherein the moveable ramp single lumen over the wire (OTW) retrograde catheter 700 may be backloaded onto a retrograde guide wire 310 that that has been positioned in the vessel 2200. In use, the distal end of the moveable ramp single lumen OTW retrograde catheter 700 is fed onto the retrograde guide wire 310 proximal end, as the retrograde guide wire 310 proximal end encounters the moveable ramp 720, the moveable ramp 720 is deflected or moved at the hinge region 740 and moves toward and/or closes over the exit port 360. The guide wire continues through the moveable ramp guide wire lumen 710 and out of the moveable ramp hub 730.

When in position for the advancing the rendezvous guide wire from the exit port 360 through the window 990, the retrograde guide wire 310 is retracted out of the proximal end of the moveable ramp single lumen OTW retrograde catheter 700. As the distal end of the retrograde guide wire 310 moves proximal of the exit port 360, the moveable ramp 720 changes position to an open position to provide a ramp configuration as illustrated in FIG. 7. When the rendezvous guide wire 2000 is introduced through the moveable ramp hub 730 and advanced through the moveable ramp guide wire lumen 710, it reaches the moveable ramp 720, which is in the open position, and the rendezvous guide wire 2000 is directed out of the exit port 360.

The moveable ramp single lumen shaft 750 may be directly connected to the moveable ramp hub 730 or incorporate a moveable ramp pigtail 760 or similar connection.

The moveable ramp 720 may be formed from the same or similar material as the moveable ramp single lumen shaft 750 and/or moveable ramp distal shaft 770 or it may be made from an entirely different material (e.g. a polymer moveable ramp single lumen shaft 750 and moveable ramp distal shaft 770 and a nitinol moveable ramp 720). The moveable ramp 720 may be constructed as part of the shaft with a living hinge or it may be a separate component added to the shaft in the hinge region 740, or some combination thereof.

The moveable ramp single lumen shaft 750 and moveable ramp distal shaft 770 are constructed to provide for suitable pushability (longitudinal motion and torquability (rotational motion) such that the moveable ramp single lumen OTW retrograde catheter 700 can be steered, rotated, and advanced within a vessel 2200 as well as enable blunt or cutting dissection through a vessel 2200 wall or through an occlusion 2300 or both. The moveable ramp single lumen shaft 750 and moveable ramp distal shaft 770 can be constructed of one or more polymers, but may include composites, metals, or combinations thereof. Examples include polyethylene, polyurethane, nylon, Pebax, polyimide, fluoropolymers, etc., moveable ramp single lumen shaft 750 and moveable ramp distal shaft 770, as well as any part of a retrograde catheter may include one or more coils, braiding, laminated sections, etc., to enhance the properties as previously described for the OTWR multilumen shaft 660. Additionally, the moveable ramp single lumen shaft 750 and moveable ramp distal shaft 770 may be constructed to enhance visualization through radiopacity and/or echogenicity as previously described for the OTWR multilumen shaft 660.

At least a portion of the moveable ramp distal shaft 770 and optionally at a portion of the moveable ramp single lumen shaft 750 are configured to enable dissection through a vessel 2200 wall, such as subintimal, intimal, intramural (medial), subadventitial, through the occlusion 2300 itself, within the vessel lumen 2210, or a combination thereof. To facilitate orientation within the vessel 2200, alignment with an antegrade catheter, and dissection, a portion of the moveable ramp single lumen shaft 750 and moveable ramp distal shaft 770 may be rounded or non-round shaped such as a flattened, spatulated, crescent shape, edgy, etc. The distal tip and/or some or all of the length of the moveable ramp distal shaft 770 may be comparatively very hard so as to not collapse on the retrograde guide wire 310 or longitudinally deform, compress, or buckle when being advanced through tissue or an occlusion 2300. The distal tip or adjacent region of the moveable ramp distal shaft 770 may contain a tip dissection feature 510 which may be blunt, facilitating blunt dissection, or cutting or edgy to create the dissection plane. The tip dissection feature 510 may be the shape of the feature, such as a radiused surface or a sharp edged tip in one or more planes, examples being 1) a radius in the plane of the exit port 360 and a shape edge perpendicular to that, similar to a radiused wedge with a sharp edge, or 2) a radius in both of these planes forming a more blunt tip dissection feature 510. The tip dissection feature 510 may be formed in the moveable ramp distal shaft 770 and/or added to the moveable ramp distal shaft 770 as a separate component, such as a metallic or polymer element, or made as part of a tip radiopaque marker, which may extend out to the distal tip. The distal tip or adjacent region of the moveable ramp distal shaft 770 may contain a retrograde tip radiopaque marker 440, made from metals such as gold, platinum, iridium, tantalum, or combinations thereof and be shaped to include the tip dissection feature 510. The distal tip or adjacent region of the moveable ramp distal shaft 770 may be made from a polymer containing a radiopaque substance, such as barium sulfate, or the entire moveable ramp distal shaft 770 may be radiopaque and/or echogenic.

The moveable ramp single lumen OTW retrograde catheter 700 contains a moveable ramp 720. The moveable ramp 720 is located in the distal region of the moveable ramp single lumen OTW retrograde catheter 700 and is used to direct the rendezvous guide wire 2000 from travelling substantially longitudinal (parallel) to the length of the moveable ramp single lumen shaft 750 to an angle with respect to the longitudinal axis of the moveable ramp single lumen shaft 750 as it exits the exit port 360. The angle the rendezvous guide wire 2000 exits the exit port 360 can influence the ability to penetrate any tissue (e.g. intima 2220) and enter a window 990, as does the shape of the tip of the rendezvous guide wire 2000. A preferable angle is 5 degrees to 80 degrees off axis, more preferably an angle of 20 degrees to 60 degrees. The moveable ramp 720 may be a flat, radiused/curved shape, any configuration that changes the angle away from the longitudinal axis of the moveable ramp single lumen shaft 750 as the rendezvous guide wire 2000 exits the exit port 360.

The moveable ramp 720 or that section of the moveable ramp single lumen OTW retrograde catheter 700 may have a different radiopacity or echogenicity than other portions of the moveable ramp single lumen OTW retrograde catheter 700. It is preferable to visualize the longitudinal and lateral position of the exit port 360 as well as rotational orientation of the exit port 360. FIG. 5A illustrates an embodiment of an exit port radiopaque marker 470 that provides for longitudinal, lateral, and rotational identification of the exit port 360 that may be employed on the moveable ramp single lumen OTW retrograde catheter 700. When viewed in the orientation in FIG. 5A, the exit port 360 can be seen as a notch 480 in the exit port radiopaque marker 470, as well as the chevrons 490 can be visualized. Looking at FIG. 5B, when rotated 90 degrees, neither the notch 480 is visible as such nor are the full chevrons 490.

Other embodiments of the exit port radiopaque marker 470 that may be employed on the moveable ramp single lumen OTW retrograde catheter 700 can be seen in FIGS. 5C-E with rotational alignment features, or simply a notch 480 without any other additional features.

In other embodiments of the exit port radiopaque marker 470, the exit port radiopaque marker 470 can be made of two or more individual radiopaque markers. Both radiopaque markers can be used as described above or one radiopaque marker can be used for identifying orientation in one rotational alignment and the other radiopaque marker for identifying rotational alignment in a different orientation.

The moveable ramp 720 or that section of the moveable ramp single lumen OTW retrograde catheter 700 can contain a reinforcement 424, such as with a metal, fiber, or polymer element; braid; coil; safety wire/strap/cable, etc., or made as a separate component and/or integrated into the moveable ramp single lumen OTW retrograde catheter 700. This is to provide an additional level of safety that the moveable ramp distal shaft and the proximal shaft region never separate. This can also provide for reducing the propensity for the moveable ramp single lumen OTW retrograde catheter 700 to deform, buckle, or kink in this region. The moveable ramp 720 or that section of the moveable ramp single lumen OTW retrograde catheter 700 reinforcement may be constructed separately or as part of the exit port radiopaque marker 470 or other longitudinal and/or lateral and/or rotational marker (radiopaque and/or echogenic).

As described for use with a 0.014″ diameter retrograde guide wire 310 and a 0.014″ rendezvous guide wire 2000, the moveable ramp single lumen OTW retrograde catheter 700 is typically within the range of from about 65 cm to about 135 cm in working length—from the distal tip to the distal end region of the moveable ramp hub 730, and for example may be about 65 cm, 80 cm, or about 135 cm depending upon the vascular access point, the location of the occlusion 2300, and intended clinical performance. The length of the moveable ramp distal shaft 770 can be from 1 cm to 10 cm or more. More preferably, the length of the moveable ramp distal shaft 770 is from 1 cm to 3 cm. The moveable ramp single lumen shaft 750 and moveable ramp distal shaft 770 are nominally 0.017″ in inside diameter and may be smaller at the distal tip, e.g. 0.015″, so as to have a tighter fit to the guide wire facilitating dissection/occlusion crossing/penetration without allowing tissue or occlusive material into the annular area between the guide wire and the moveable ramp distal shaft 770. The short axis (if not round) wall thickness of the moveable ramp distal shaft 770 is typically from 0.0015″ to 0.0200″ and may taper or blend to an edge. More preferably, the short axis wall thickness of the moveable ramp distal shaft 770 is from 0.003″ to 0.0100″ and may taper or blend to an edge. This results in a moveable ramp distal shaft 770 short axis profile 3.0 Fr or less, or more preferably 2.6 Fr or less.

Portions or all of the moveable ramp single lumen OTW retrograde catheter 700 may be coated to enhance reflectivity, increase lubricity, increase stiffness, etc. Examples of coatings include a lubricious coating such as silicone or fluoropolymer, hydrophilic, hydrophobic, etc., on the outside of the moveable ramp distal shaft 770 and optionally at a portion of the moveable ramp single lumen shaft 750 and moveable ramp 720 section of the shaft to reduce the force needed to achieve dissection/occlusion crossing/penetration. A polymer surface coating can be applied to the moveable ramp 720 or that section of the moveable ramp single lumen OTW retrograde catheter 700 to further increase the reflectivity of that area of the moveable ramp single lumen OTW retrograde catheter 700. An internal or external coating (or laminate/layer) of polyimide with a Pebax would increase stiffness and pushability of the moveable ramp single lumen shaft 750 and/or moveable ramp distal shaft 770.

An example of an 80 cm overall length moveable ramp single lumen OTW retrograde catheter 700 is for use with a 0.014″ rendezvous guide wire 2000 is as follows. Moveable ramp distal shaft 770 having a length of 2 cm, the moveable ramp guide wire lumen in this region tapering (or stepped) from 0.017″ proximally to 0.015″ at the distal end. The moveable ramp distal shaft 770 having a having a spatulated, slightly concave, cross-section (similar to FIG. 4C) with a short axis wall thickness of 0.005″ for a short axis profile of 0.025″ to 0.027″ (1.9 to 2.1 Fr), and a long axis wall thickness of 0.0085″ for a profile of 0.034″ (2.6 Fr). The moveable ramp distal shaft 770 comprised of Pebax 72D loaded with 40% BaSO₄ for radiopacity. A retrograde tip radiopaque marker 440 being 1 mm in length comprised of 90% Pt 10% Ir with a wall thickness of 0.002″, located 1 mm proximal of the distal end. A tip dissection feature 510 being bullet shaped when viewed perpendicular to the long axis section and edgy when viewed perpendicular to the sort axis section. The moveable ramp distal shaft 770 having a 10-degree angulation in the opposite direction of the exit port 360, effectively being a 10-degree angulation from the longitudinal axis of the moveable ramp single lumen shaft 750.

The moveable ramp single lumen shaft 750 having a moveable ramp wire lumen 710 with an inside diameter of 0.017″ throughout its length. The moveable ramp single lumen shaft 750 having a round cross-section constructed with an inner liner 422 of Pebax 35D to 72D (more preferably 55D), a 304V stainless steel braid over an inner liner of Pebax 55D with 8 to 32 wires (more preferably 16 wires), 0.001″ to 0.003″ diameter round or flat (0.0005″ to 0.0020″ thick by 0.001″ to 0.005″ wide wires) (more preferably 0.002″ round), 30 to 50 pics per inch (more preferably 40 pics per inch), with an outer jacket of Pebax 55 to 72D (more preferably 72D). The overall moveable ramp single lumen shaft 750 wall thickness of 0.0085″ for an outside diameter of 0.034″ (2.6 Fr).

A moveable ramp 720, constructed as part of the moveable ramp distal shaft 770 by cutting (with a laser, sharp instrument, etc.) or other methods to create a moveable ramp 720 with a hinge. The moveable ramp 720 is set in the open position (as shown in FIG. 7) prior to advancing the moveable ramp single lumen OTW retrograde catheter 700 over a retrograde guide wire 310. The moveable ramp 720 having a length of 0.030″ and a width of 0.017″. A moveable ramp radiopaque marker 780, similar to that shown in FIGS. 5A-5B, made of 90% Pt 10% Ir with a wall thickness of 0.002″, and overall length of 0.130″ with a notch 480 length of 0.050″.

A moveable ramp hub 730 adjacent the proximal end of the moveable ramp single lumen shaft 750. The moveable ramp hub 730 being a Luer fitting with an internal taper to form a smooth transition from the internal surface of the Luer fitting to the moveable ramp wire lumen 710. A visual hub marker 370 on the moveable ramp hub 730 aligned with the moveable ramp 720 and a stripe 500 on the moveable ramp single lumen shaft 750 continuing from the hub marker 370 running to and aligned with the moveable ramp 720.

The distal 15 cm of the moveable ramp single lumen OTW retrograde catheter 700 having an external hydrophilic coating for improved lubricity.

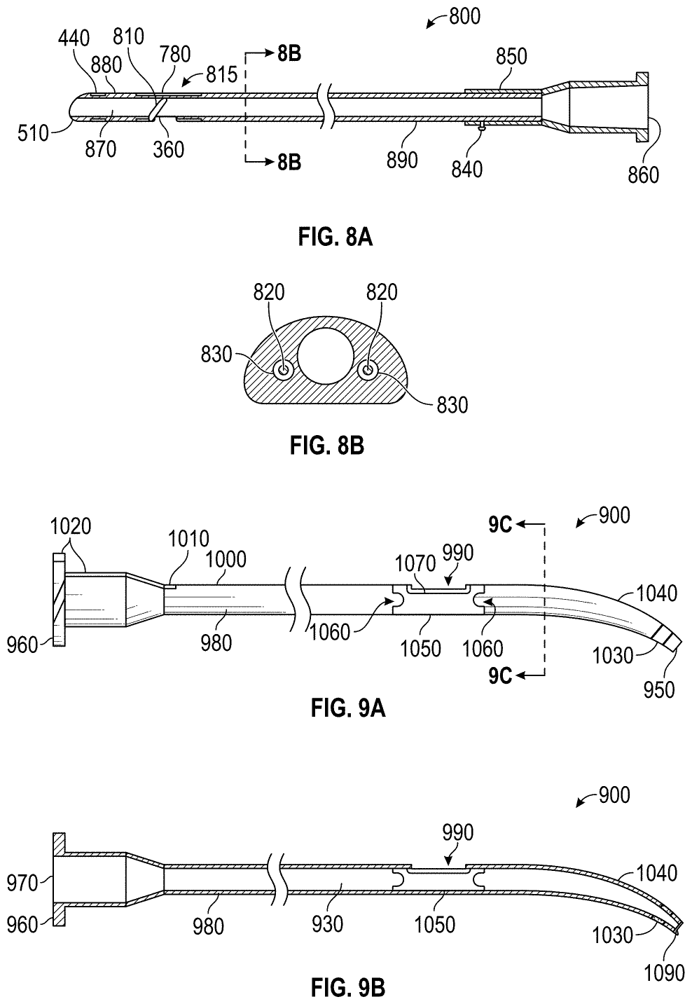

FIG. 8A illustrates an embodiment of a single guide wire lumen over the wire retrograde catheter with a manually actuated moveable ramp 810 in the ramp open position—a manually actuated moveable ramp OTW retrograde catheter 800. Having a manually actuated moveable ramp allows the user to remove the retrograde guide wire 310 and if so desired, reinsert the retrograde guide wire 310 without having to remove the manually actuated moveable ramp OTW retrograde catheter 800 from the patient/vessel 2200. This may be necessary if using a single guide wire lumen configuration and achieving successful rendezvous is not achieved and it is desired to reposition the single lumen retrograde catheter with the retrograde guide wire 310 in a position distal of the moveable ramp (710, 810).

The manually actuated moveable ramp 810 can be actuated, for example, by one or more pull wires 820 which travel in one or more pull wire lumens 830, as illustrated in FIG. 8B or other mechanical actuators. The one or more pull wires 820 can be connected to a slide 840 which resides in a slide hub 850. Moving the slide 840 moves the one or more pull wires 820 to cause the manually actuated moveable ramp 810 to move to the closed position—where the retrograde guide wire 310 can be moved freely with manually actuated moveable ramp guide wire lumen 870. The slide hub 850 may also contain or be separate from the hub fitting 860 (e.g. in communication with the slide hub 850 using a pigtail), which contains a Luer fitting or tapered section and enables access to the manually actuated moveable ramp guide wire lumen 870.

In alternative embodiments, the manually actuated moveable ramp 810 can be thermally/electrically actuated, for example supplying a current to the ramp actuator, inducing heat or a thermal change such as for use with shape memory metals (e.g. nitinol), etc. In embodiments where a supply of electrical energy is required, the slide hub 850 may be replaced by a hub with a connection to a power source, or the power source may be located on or within the hub or proximal region of the manually actuated moveable ramp OTW retrograde catheter 800.

The manually actuated moveable ramp single lumen shaft 880 and manually actuated moveable ramp multilumen shaft 890 are constructed to provide for suitable pushability (longitudinal motion) and torquability (rotational motion) such that the manually actuated moveable ramp OTW retrograde catheter 800 can be steered, rotated, and advanced within a vessel 2200 as well as enable blunt or cutting dissection through a vessel 2200 wall or through an occlusion 2300 or both. The manually actuated moveable ramp multilumen shaft 890 is typically semi-circular, round, oval, crescent shape, or edgy in cross-section and can be constructed of one or more polymers, composites, metals, or combinations thereof. Examples include polyethylene, polyurethane, nylon, Pebax, polyimide, fluoropolymers, carbon fiber, stainless steel, Nitinol, titanium, etc. The manually actuated moveable ramp multilumen shaft 890 can be made at least in part as a laminate of various materials. The manually actuated moveable ramp multilumen shaft 890 as constructed with allow for lateral motion control as well. The manually actuated moveable ramp multilumen shaft 890 may be constructed to be radiopaque by adding a radiopaque material in a polymer construction, such as barium sulfate or tantalum or a radiopaque reinforcement 424 (e.g. braid, coil), or by forming it from a metal or a coated metal (e.g. gold-coated stainless steel), or a combination thereof. Also, the manually actuated moveable ramp multilumen shaft 890 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the manually actuated moveable ramp OTW retrograde catheter 800 using fluoroscopy and ultrasound to better improve positioning and orientation.

The manually actuated moveable ramp multilumen shaft 890, as well as any part of a retrograde catheter may include a reinforcement 424, one or more coils, braiding, etc., to enhance the properties. For example, a braid or braided section within the manually actuated moveable ramp multilumen shaft 890 would enhance torquability and a coil within the manually actuated moveable ramp multilumen shaft 890 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

As illustrated in FIG. 4A, similar visual and/or tactile indicators can be used on the manually actuated moveable ramp multilumen shaft 890 and/or slide hub 850 for indicating the rotational orientation of the exit port 360 or other more distal manually actuated moveable ramp OTW retrograde catheter 800 element. For example, slide hub 850 and/or the manually actuated moveable ramp multilumen shaft 890 may have a visual indicator of the rotational orientation of the exit port 360. The manually actuated moveable ramp multilumen shaft 890 may have a stripe 500 as a visual indicator to provide visual feedback and/or raised and/or textured to provide tactile feedback of the rotational orientation of the exit port 360.

The manually actuated moveable ramp single lumen shaft 880 is constructed to provide for pushability (longitudinal motion) and torquability (rotational motion) such that the manually actuated moveable ramp OTW retrograde catheter 800 can be steered, rotated, and advanced within a vessel 2200 as well as enable dissection through a vessel 2200 wall or through an occlusion 2300 or both. The manually actuated moveable ramp single lumen shaft 880 can be constructed of one or more polymers, but may include composites, metals, or combinations thereof. Examples include polyethylene, polyurethane, nylon, Pebax, polyimide, fluoropolymers, etc. The manually actuated moveable ramp single lumen shaft 880, as well as any part of a retrograde catheter may include a reinforcement 424, one or more coils, braiding, laminated sections, etc., to enhance the properties as previously described for the on the manually actuated moveable ramp multilumen shaft 890. Additionally, the manually actuated moveable ramp single lumen shaft 880 may be constructed to enhance visualization through radiopacity and/or echogenicity as previously described for the manually actuated moveable ramp multilumen shaft 890.

At least a portion of the manually actuated moveable ramp single lumen shaft 880 and optionally at least a portion of the manually actuated moveable ramp multilumen shaft 890 is configured to enable dissection through a vessel 2200 wall, such as subintimal, intimal, intramural (medial), subadventitial, through the occlusion 2300 itself, within the true vessel lumen 2210, or a combination thereof. To facilitate orientation within the vessel 2200, alignment with an antegrade catheter, and dissection, a portion of the manually actuated moveable ramp single lumen shaft 880 may be round or non-round shaped such as a flattened, spatulated, semi-circular, oval, crescent shape, edgy, etc. The distal tip and/or some or all of the length of the manually actuated moveable ramp single lumen shaft 880 may be comparatively very hard so as to not collapse on the retrograde guide wire 310 or longitudinally deform, compress, or buckle when being advanced through tissue or an occlusion 2300. The distal tip or adjacent region of the manually actuated moveable ramp single lumen shaft 880 may contain a tip dissection feature 510 which may be blunt, facilitating blunt dissection, or cutting or edgy to create the dissection plane. The tip dissection feature 510 may be the shape of the feature, such as a radiused surface or a sharp edged tip in one or more planes, examples being 1) a radius in the plane of the exit port 360 and a shape edge perpendicular to that, similar to a radiused wedge with a sharp edge, or 2) a radius in both of these planes forming a more blunt tip dissection feature 510. The tip dissection feature 510 may be formed in the manually actuated moveable ramp single lumen shaft 880 and/or added to the manually actuated moveable ramp single lumen shaft 880 as a separate component, such as a metallic or polymer element, or made as part of a tip radiopaque marker, which may extend out to the distal tip. The distal tip or adjacent region of the manually actuated moveable ramp single lumen shaft 880 may contain a retrograde tip radiopaque marker 440, made from metals such as gold, platinum, iridium, tantalum, or combinations thereof and be shaped to include the tip dissection feature 510. The distal tip or adjacent region of the manually actuated moveable ramp single lumen shaft 880 may contain a retrograde tip radiopaque marker, made from metals such as gold, platinum, iridium, tantalum, or combinations thereof or the distal tip may be made from a polymer containing a radiopaque substance, such as barium sulfate, or the entire manually actuated moveable ramp single lumen shaft 880 and/or manually actuated moveable ramp multilumen shaft 890 may be radiopaque and/or echogenic.

The manually actuated moveable ramp OTW retrograde catheter 800 contains a manually actuated moveable ramp section 815. The manually actuated moveable ramp 810 is used to direct the rendezvous guide wire 2000 from travelling substantially longitudinal (parallel) to the length of the manually actuated moveable ramp multilumen shaft 890 to an angle with respect to the longitudinal axis of the manually actuated moveable ramp multilumen shaft 890 as it exits the exit port 360. The angle the rendezvous guide wire 2000 exits the exit port 360 can influence the ability to penetrate any tissue (e.g. intima 2220) and enter a window 990, as does the shape of the tip of the rendezvous guide wire 2000. A preferable angle is 5 degrees to 80 degrees off axis, more preferably an angle of 20 degrees to 60 degrees. The manually actuated moveable ramp 810 may be a flat, radiused/curved shape, any configuration that changes the angle away from the longitudinal axis of the manually actuated moveable ramp 810 as the rendezvous guide wire 2000 exits the exit port 360.

The manually actuated moveable ramp 810 may be formed from the same or similar material as the manually actuated moveable ramp multilumen shaft 890 and/or manually actuated moveable ramp single lumen shaft 880 or it may be made from an entirely different material (e.g. a polymer manually actuated moveable ramp multilumen shaft 890 and manually actuated moveable ramp single lumen shaft 880 and a nitinol manually actuated moveable ramp 810). The manually actuated moveable ramp 810 may be constructed as part of the shaft with a living hinge or it may be a separate component added to the shaft.

The manually actuated moveable ramp section 815 may have a different radiopacity or echogenicity than other portions of the manually actuated moveable ramp OTW retrograde catheter 800. It is preferable to visualize the longitudinal and lateral position of the exit port 360 as well as rotational orientation of the exit port 360. FIG. 5A illustrates one embodiments of an exit port radiopaque marker 470 that provides for longitudinal, lateral, and rotational identification of the exit port 360 that may be employed on the manually actuated moveable ramp OTW retrograde catheter 800. When viewed in the orientation in FIG. 5A, the exit port 360 can be seen as a notch 480 in the exit port radiopaque marker 470, as well as the chevrons 490 can be visualized. Looking at FIG. 5B, when rotated 90 degrees, neither the notch 480 is visible as such nor are the full chevrons 490.

Other embodiments of the exit port radiopaque marker 470 that may be employed on the manually actuated moveable ramp OTW retrograde catheter 800 can be seen in FIGS. 5C-E with rotational alignment features, or simply a notch 480 without any other additional features.

In other embodiments of the exit port radiopaque marker 470, the exit port radiopaque marker 470 can be made of two or more individual radiopaque markers. Both radiopaque markers can be used as described above or one radiopaque marker can be used for identifying orientation in one rotational alignment and the other radiopaque marker for identifying rotational alignment in different orientation.

The manually actuated moveable ramp section 815 can contain a reinforcement 424, such as with a metal, fiber, or polymer element; braid; coil; safety wire/strap/cable, etc., or made as a separate component and/or integrated into the manually actuated moveable ramp OTW retrograde catheter 800. This is to provide an additional level of safety that the manually actuated moveable ramp single lumen shaft 880 and manually actuated moveable ramp multilumen shaft 890 never separate. This can also provide for reducing the propensity for the manually actuated moveable ramp OTW retrograde catheter 800 to deform, buckle, or kink in this region. The manually actuated moveable ramp section 815 reinforcement may be constructed separately or as part of the exit port radiopaque marker 470 or other longitudinal and/or lateral and/or rotational marker (radiopaque and/or echogenic).

As described for use with a 0.014″ diameter retrograde guide wire 310 and a 0.014″ rendezvous guide wire 2000, the manually actuated moveable ramp OTW retrograde catheter 800 is typically within the range of from about 65 cm to about 135 cm in working length—from the distal tip to the distal end of the slide hub 850, and for example may be about 65 cm, 80 cm, or about 135 cm depending upon the vascular access point, the location of the occlusion 2300, and intended clinical performance. The length of the manually actuated moveable ramp single lumen shaft 880 can be from 1 cm to 10 cm or more. More preferably, the length of the manually actuated moveable ramp single lumen shaft 880 is from 1 cm to 3 cm. The manually actuated moveable ramp multilumen shaft 890 short axis (if not round) wall thickness is from 0.0015″ to 0.0200″. More preferably, the short axis wall thickness of the manually actuated moveable ramp multilumen shaft 890 is from 0.003″ to 0.0100″. This results in a manually actuated moveable ramp multilumen shaft 890 short axis profile 3.0 Fr or less, or more preferably 2.6 Fr or less. The long axis is from 0.027″ to 0.060″, more preferably from 0.027″ to 0.040″. The guide wire lumen in the manually actuated moveable ramp single lumen shaft 880 is nominally 0.017″ in inside diameter and may be smaller at the distal tip, e.g. 0.015″, so as to have a tighter fit to the guide wire facilitating dissection/occlusion crossing/penetration without allowing tissue or occlusive material into the annular area between the guide wire and the manually actuated moveable ramp single lumen shaft 880. The short axis (if not round) wall thickness of the manually actuated moveable ramp single lumen shaft 880 is from 0.0015″ to 0.0200″ and may taper or blend to an edge. More preferably, the short axis wall thickness of the manually actuated moveable ramp single lumen shaft 880 is from 0.003″ to 0.0100″. This results in a manually actuated moveable ramp single lumen shaft 880 short axis profile 3.0 Fr or less, or more preferably 2.6 Fr or less.

Portions or all of the manually actuated moveable ramp OTW retrograde catheter 800 may be coated to enhance reflectivity, increase lubricity, increase stiffness, etc. Examples of coatings include a lubricious coating such as silicone or fluoropolymer, hydrophilic, hydrophobic, etc., on the outside of the manually actuated moveable ramp single lumen shaft 880 and OTWR ramp section 680 to reduce the force needed to achieve dissection/occlusion crossing/penetration. A polymer surface coating can be applied to the manually actuated moveable ramp section 815 to further increase the reflectivity of that area of the manually actuated moveable ramp OTW retrograde catheter 800. An internal or external coating (or laminate/layer) of polyimide with a Pebax would increase stiffness and pushability of the manually actuated moveable ramp multilumen shaft 890 and/or manually actuated moveable ramp single lumen shaft 880.

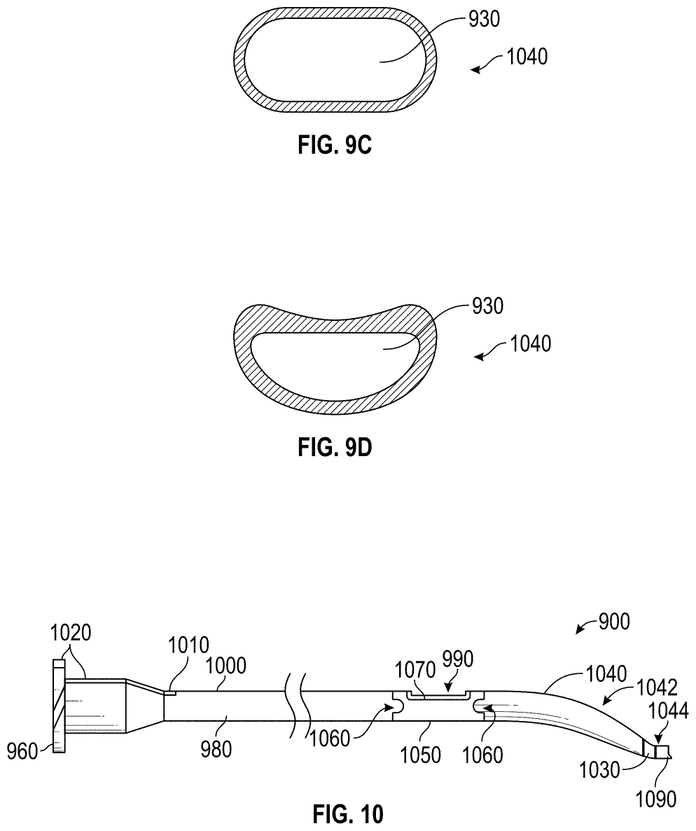

FIGS. 9A-9D depict a single lumen antegrade catheter of the present invention. The antegrade catheter is particularly designed to align with a retrograde catheter and receive the rendezvous guide wire 2000 and enable the rendezvous guide wire 2000 to advance out of the patient at the antegrade access site 910, such as out of the antegrade catheter hub. It will be understood that though the antegrade catheter and methods are described primarily with reference to lower extremity vascular procedures for receiving a rendezvous guide wire 2000, the antegrade catheter can also be used in treatments for other parts of the body.

The antegrade catheter will be described sized for use in the lower extremities over a 0.035″ guide wire, though other sizes, lengths, and diameters of the antegrade catheter and guide wire are within the scope of the invention. As illustrated in FIGS. 9A-9D, the antegrade catheter may be designed in a single lumen configuration—a single lumen antegrade catheter 900. The antegrade single lumen 930 serves to both allow over the wire functionality by using an antegrade guide wire 940 in the same lumen that will receive the rendezvous guide wire 2000. The single lumen antegrade catheter 900 has an antegrade single lumen hub 960 with an antegrade single lumen hub opening 970 or port which serves as an entry port for the antegrade guide wire 940 and an exit for the rendezvous guide wire 2000. The antegrade single lumen hub 960 consists of a Luer fitting or tapered section. The antegrade single lumen hub 960 may be attached to the antegrade single lumen shaft 980 such as by bonding, ultrasonic welding, or may be molded onto the antegrade single lumen shaft 980. The antegrade single lumen hub 960 is typically plastic, such as polyethylene, polyurethane, polycarbonate, etc., or may be metallic or composite or a combination thereof.

The antegrade single lumen shaft 980 is constructed to provide for suitable pushability (longitudinal motion) and torquability (rotational motion) such that the single lumen antegrade catheter 900 can be steered, rotated, and advanced within a vessel 2200 and aligned with a retrograde catheter. The antegrade single lumen shaft 980 can be constructed of one or more polymers, composites, metals, or combinations thereof. Examples include polyethylene, polyurethane, nylon, Pebax, polyimide, fluoropolymers, carbon fiber, stainless steel, nitinol, titanium, etc. The antegrade single lumen shaft 980 can be made at least in part as a laminate of various materials. The antegrade single lumen shaft 980 as constructed with allow for lateral motion control as well. The antegrade single lumen shaft 980 may be constructed to be radiopaque by adding a radiopaque material in a polymer construction, such as barium sulfate or tantalum or a radiopaque reinforcement 424 (e.g. braid, coil), or by forming it from a metal or a coated metal (e.g. gold-coated stainless steel), or a combination thereof. Also, the antegrade single lumen shaft 980 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the single lumen antegrade catheter 900 using fluoroscopy and ultrasound to better improve positioning and orientation.

The antegrade single lumen shaft 980 may include a reinforcement 424, one or more coils, braiding, etc., to enhance the properties. For example, a braid or braided section within the antegrade single lumen shaft 980 would enhance torquability and a coil within the antegrade single lumen shaft 980 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

The antegrade single lumen shaft 980 and/or antegrade single lumen hub 960 may have visual and/or tactile indicators as an indicium of the rotational orientation of the window 990 or other more distal mini-rail single lumen antegrade catheter 900 element/feature. For example, the antegrade single lumen hub 960 may have an antegrade hub marker 1020 while the antegrade single lumen shaft 980 may have an antegrade stripe 1000 and/or antegrade shaft marker 1010 as a visual indicator to provide visual feedback and/or raised and/or textured to provide tactile feedback of the rotational orientation of the window 990.

The antegrade distal shaft region 1040 may employ a section with one or more curves or bends to help steer the catheter through the vasculature as well as position the window 990 against the vessel 2200 wall. The distal tip region of the antegrade distal shaft region 1040 that is not aligned with (e.g. curved or bent away from) the longitudinal axis of the proximal region/shaft pushes against the vessel 2200 wall opposite of the side of the window 990, thus pushing the window 990 up to the vessel 2200 wall. Typical angle of the curve can be 5 degrees to 60 degrees, more preferably 10 degrees to 20 degrees. An additional curve or bend may be included in the antegrade distal shaft region 1040 as illustrated in FIG. 10 includes a first antegrade distal shaft region 1042, bent of curved away from the longitudinal axis of the antegrade single lumen shaft 980, and a second antegrade distal shaft region 1044 bent of curved with respect to the longitudinal axis of the first antegrade distal shaft region 1042. To facilitate orientation within the vessel 2200 and alignment with a retrograde catheter and exit port 360, the antegrade distal shaft region 1040 is configured to be complimentary to the distal shaft region of a retrograde catheter and may be round or non-round shaped such as a flattened, oval, spatulated, crescent shape, edgy, etc.

The antegrade distal shaft region 1040 or antegrade distal tip 1080 may have a dissection feature 1090 or shape (e.g. a stiff and/or sharp section, a protrusion or depression, add on element, longitudinally and/or radially extending) that enables it to be used as a remote surgical tool or device to assist in creating a dissection, micro-dissection, tissue disruption, or passage through any tissue (e.g. intima 2220), that may be present between the vessel lumen 2210 and the exit window 360 of a retrograde catheter, as well as to manipulate or cut tissue, separate plaque from the artery, and/or separate layers of artery wall. During insertion and movement of the mini-rail single lumen antegrade catheter 900 over the antegrade guide wire 940, the curvature or bend/deflection of the antegrade distal shaft region 1040 will be somewhat reduced by the stiffness of the antegrade guide wire 940, thus protecting the vessel 2200 from damage during this movement. An additional bend in the antegrade distal shaft region 1040 may also facilitate vessel 2200 protection during insertion, advancement, and removal of the single lumen antegrade catheter 900. When in position as described, the antegrade guide wire 940 is retracted and/or positioned to achieve a certain deflection of the antegrade distal shaft region 1040 to engage the dissection feature 1090 against the vessel 2200 wall and manipulated (e.g. moving longitudinally, laterally, a combination thereof) to disrupt the tissue.

The distal tip region or adjacent region of the antegrade single lumen shaft 980 may contain an antegrade tip radiopaque marker 1030, made from metals such as gold, platinum, iridium, tantalum, or combinations thereof or the distal tip region may be made from a polymer containing a radiopaque substance, such as barium sulfate, or the antegrade single lumen shaft 980 may be radiopaque and/or echogenic. The antegrade tip radiopaque marker 1030 could be shaped to form the dissection feature 1090.

The dissection feature 1090 may be provided on any of the antegrade catheters disclosed herein, depending upon desired clinical performance.

The region of the antegrade single lumen shaft 980 that contains the window 990, may have a different radiopacity or echogenicity than other portions of the single lumen antegrade catheter 900. It is preferable to visualize the longitudinal and lateral position of the window 990 as well as rotational orientation of the window 990. FIGS. 9A-B illustrate embodiments of a window radiopaque marker 1050 with antegrade notch 1070 that provides for longitudinal, lateral, and rotational identification of the window 990. When viewed in the orientation in FIGS. 9A-B, the window 990 can be seen as an antegrade notch 1070 in the window radiopaque marker 1050, as well as the semi-circles 1060 can be visualized. When rotated 90 degrees, neither the antegrade notch 1070 is visible as such nor are the semi-circles 1060.

Other embodiments of a window radiopaque marker 1050 can be seen in FIGS. 5C-E with rotational alignment features, or simply a notch 480 without any other additional features.

In other embodiments, the window radiopaque marker 1050 can be positioned on the inside of the antegrade single lumen shaft 980 as illustrated in FIG. 9B. By having the window radiopaque marker 1050 on the inside in the window 990 region, this can serve to protect the inside of the antegrade single lumen shaft 980 when the rendezvous guide wire 2000 enters through the window 990, especially if the rendezvous guide wire 2000 incorporates an obstruction or tissue piercing tip and a piercing tip is used with the single lumen antegrade catheter 900 in position for rendezvous. Alternatively, the window radiopaque marker 1050 can remain on the outside of the antegrade single lumen shaft 980 and the inside of the antegrade single lumen shaft 980 can be reinforced in this region to prevent damage from the rendezvous guide wire 2000. Reinforcement can be a tube, shaft liner, semi-circular tube, strip, insert, etc., made of material with one or more of the following characteristics, including abrasion/pierce/cut/wear resistance (e.g. metal, polymer) and positioned on/in one portion or side of the antegrade single lumen shaft 980 or throughout. Any of the positions of the window radiopaque marker 1050 and/or reinforcement disclosed herein may be used on any of the antegrade catheters disclosed herein, depending upon the desired clinical performance.

In other embodiments of the window radiopaque marker 1050, the window radiopaque marker 1050 can be made of two or more individual radiopaque markers. Both radiopaque markers can be used as described above or one radiopaque marker can be used for identifying orientation in one rotational alignment and the other radiopaque marker for identifying rotational alignment in a different orientation.

The region of the antegrade single lumen shaft 980 that contains the window 990 can contain a reinforcement 424, such as with a metal, fiber, or polymer element; braid; coil; safety wire/strap/cable, etc., or made as a separate component and/or integrated into the single lumen antegrade catheter 900. This is to provide an additional level of safety that the antegrade distal shaft region 1040 and the shaft region proximal to the window 990 never separate. This can also provide for reducing the propensity for the antegrade single lumen shaft 980 to deform, buckle, or kink in this region. The window 990 region reinforcement may be constructed as part of the window radiopaque marker 1050 or other longitudinal and/or lateral and/or rotational marker (radiopaque and/or echogenic).

As described for use over a 0.035″ diameter antegrade guide wire, the single lumen antegrade catheter 900 is typically 135 cm in working length—from the distal tip to the distal end region of the antegrade single lumen hub 960, however, longer or shorter lengths may be constructed depending on the location of the occlusion 2300 and access site. The length of the antegrade distal shaft region 1040 is typically 1 mm to 50 mm or longer, more preferably from 4 mm to 20 mm. The length of the window 990 is 0.014″ to 0.8″ or longer, more preferably from 0.040″ to 0.60″, and more preferably from 0.2″ to 0.4″. The antegrade single lumen 930 is nominally 0.038″ in diameter and may be smaller at the distal tip, e.g. 0.036″, so as to have a tighter fit to the antegrade guide wire 940, however, the antegrade single lumen 930 can be constructed for any guide wire diameter or larger (e.g. 5 Fr to 7 Fr internal diameter) to ease reception of the rendezvous guide wire 2000. The short axis if not round, for example oval (FIG. 9C) or spatulated (FIG. 9D), wall thickness of the antegrade single lumen shaft 980 is typically from 0.0015″ to 0.0200″. More preferably, the short axis wall thickness of the antegrade single lumen shaft 980 is from 0.003″ to 0.010″. This results in a distal section short axis profile 4.3 Fr or less, or more preferably 3.6 Fr or less.

Portions or all of the single lumen antegrade catheter 900 may be coated to enhance reflectivity, increase lubricity, increase stiffness, etc. Examples of coatings include a lubricious coating such as silicone or fluoropolymer, hydrophilic, hydrophobic, etc., on the outside of the antegrade distal shaft region 1040 to reduce the force needed to navigate the vasculature. A polymer surface coating can be applied to the window 990 section to further increase the reflectivity of that area of the single lumen antegrade catheter 900. An internal or external coating (or laminate/layer) of polyimide with a Pebax over all or a portion of the antegrade single lumen shaft 980 would increase stiffness and pushability.

An example construction of a 135 cm overall length single lumen antegrade catheter 900 is as follows. An antegrade single lumen shaft 980 with an antegrade single lumen 930 inside diameter of 0.066″. The antegrade single lumen shaft 980 constructed with an inner liner 422 of Pebax 35D to 72D (more preferably 55D), a 304V stainless steel braid reinforcement 424 over the inner liner 422 of Pebax 55D with 8 to 32 wires (more preferably 32 wires), 0.001″ to 0.003″ diameter round or flat (0.0005″ to 0.0020″ thick by 0.001″ to 0.005″ wide wires) (more preferably 0.002″ round), 30 to 50 pics per inch (more preferably 30 pics per inch), with an outer jacket 426 of Pebax 55 to 72D (more preferably 72D) loaded with 40% BaSO₄ for radiopacity. An antegrade tip radiopaque marker 1 mm in length comprised of 90% Pt 10% Ir with a wall thickness of 0.002″, located 1 mm proximal of the antegrade single lumen distal opening 950. The antegrade single lumen shaft 980 wall thickness of 0.0085″ for an outside diameter of 0.083″ (6.3 Fr). The antegrade distal shaft region 1040 tapers down to an internal diameter of 0.038″ with an outside diameter of 0.053″ (4 Fr). The curve in the antegrade distal shaft region 1040 forming an angle of 10 degrees to 15 degrees as seen in FIGS. 9A and 9B. A dissection feature 1090 comprising a small edge or burr at the distal tip of the antegrade distal shaft region 1040.

The distal end of the window 990 being located 2 cm from the antegrade single lumen distal opening 950. The window 990 having a width of 0.066″ and a length of 0.40″. A window radiopaque marker 1050 as shown in FIGS. 9 and 5C, made of 90% Pt 10% Ir with a wall thickness of 0.002″, and overall length of 0.50″ with a window 990 length of 0.42″.

An antegrade single lumen hub 960 adjacent the proximal end of the antegrade single lumen shaft 980. The antegrade single lumen hub 960 being a Luer fitting with an internal taper to form a smooth transition from the internal surface of the Luer fitting to the antegrade single lumen 930. A visual antegrade hub marker 1020 on the antegrade single lumen hub 960 aligned with the window 990 and an antegrade stripe 1000 on the antegrade single lumen shaft 980 continuing from the antegrade hub marker 1020 running to and aligned with the window 990.

The distal 30 cm of the single lumen antegrade catheter 900 having an external hydrophilic coating for improved lubricity.

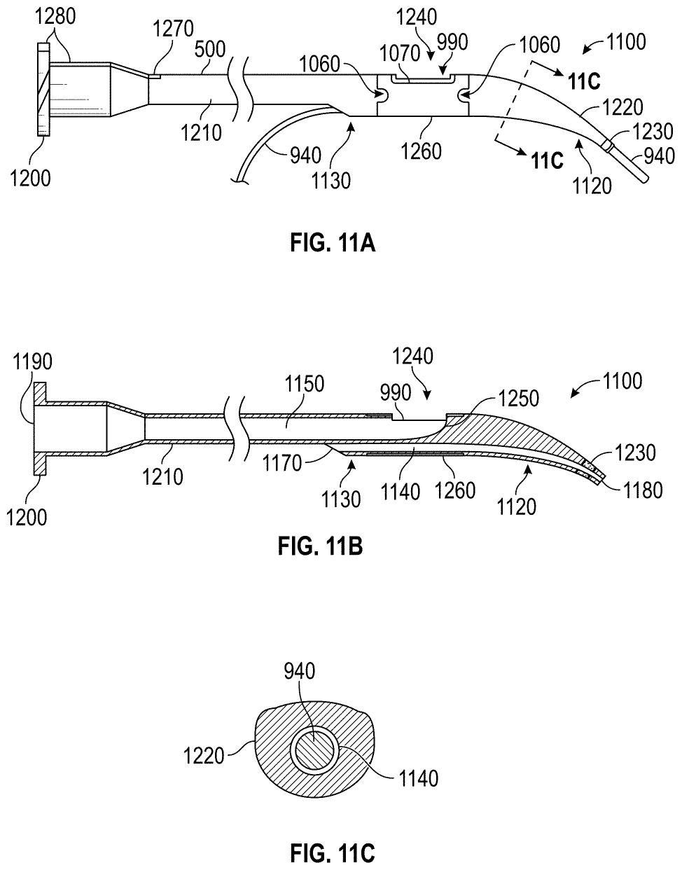

FIG. 11A illustrates an embodiment of a mini-rail antegrade catheter 1100, while FIG. 11B is a cross-section of the same catheter. The mini-rail antegrade catheter 1100 contains two lumens, an antegrade mini-rail guide wire lumen 1140 and an antegrade rendezvous guide wire lumen 1150. The antegrade mini-rail guide wire lumen 1140 has an antegrade mini-rail lumen proximal opening 1170 or port and an antegrade mini-rail lumen distal opening 1180. The antegrade rendezvous guide wire lumen 1150 has an antegrade proximal opening 1190 at the mini-rail antegrade catheter hub 1200 and a window section 1240. Within the window section 1240, there is a window 990 which serves as an entry point for receiving a rendezvous guide wire 2000 into the mini-rail antegrade catheter 1100.

The antegrade rendezvous guide wire shaft 1210 may be single lumen throughout the majority of its length and constructed to provide for suitable pushability (longitudinal motion) and torquability (rotational motion) such that the mini-rail antegrade catheter 1100 can be steered, rotated, and advanced within a vessel 2200 as well as align the window 990 with an exit port 360 of a retrograde catheter. The antegrade rendezvous guide wire shaft 1210 is typically round in cross-section and can be constructed of one or more polymers, composites, metals, or combinations thereof. Examples include polyethylene, polyurethane, nylon, Pebax, polyimide, fluoropolymers, carbon fiber, stainless steel, nitinol, titanium, etc. The antegrade rendezvous guide wire shaft 1210 can be made at least in part as a laminate of various materials. The antegrade rendezvous guide wire shaft 1210 as constructed with allow for lateral motion control as well. The antegrade rendezvous guide wire shaft 1210 may be at least in part constructed to be radiopaque by adding a radiopaque material in a polymer construction, such as barium sulfate or tantalum, or by forming it from a metal or a coated metal (e.g. gold-coated stainless steel), or a combination thereof. Also, the antegrade rendezvous guide wire shaft 1210 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the mini-rail antegrade catheter 1100 using fluoroscopy and ultrasound to better improve positioning and orientation.

The antegrade rendezvous guide wire shaft 1210, as well as any part of an antegrade catheter may contain a reinforcement 424, one or more coils, braiding, etc., to enhance the properties. For example, a braid or braided section within the antegrade rendezvous guide wire shaft 1210 would enhance torquability and a coil within the antegrade rendezvous guide wire shaft 1210 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

The antegrade rendezvous guide wire shaft 1210 and/or mini-rail antegrade catheter hub 1200 may have a visual and/or tactile indicator of the rotational orientation of the window 990 and/or a antegrade mini-rail proximal lumen opening 1170, or other more distal mini-rail antegrade catheter 1100 element. For example, the mini-rail antegrade catheter hub 1200 may have an antegrade mini-rail hub marker 1280, the antegrade rendezvous guide wire shaft 1210 may have an antegrade mini-rail shaft marker 1270 and/or a stripe 500 as a visual indicator to provide visual feedback and/or raised and/or textured to provide tactile feedback of the rotational orientation of the window 990.

The antegrade mini-rail distal region 1120 of the mini-rail antegrade catheter 1100 contains at least a portion of the antegrade mini-rail shaft 1220. A portion or the entire length of the antegrade mini-rail shaft 1220 is configured to achieve alignment with a retrograde catheter. The antegrade mini-rail distal region 1120 may employ one or more curves or bent sections to help steer the catheter through the vasculature as well as position the window 990 against the vessel 2200 wall. The distal tip region of the antegrade mini-rail distal region 1120 that is curved or bent away from the longitudinal axis of the proximal shaft pushes against the vessel 2200 wall opposite of the side of the window 990, thus pushing the window 990 up to the vessel 2200 wall. To facilitate orientation within the vessel 2200 and alignment with a retrograde catheter and exit port 360, the antegrade mini-rail distal region 1120 and/or the window section 1240 configured to be complimentary to the distal shaft region of a retrograde catheter and may be round or non-round shaped such as a flattened, spatulated, crescent shape, edgy, etc. An example of a cross-section of the antegrade mini-rail shaft 1220 is illustrated in FIG. 11C. The distal tip or adjacent region of the antegrade mini-rail shaft 1220 may contain an antegrade tip radiopaque marker 1230, made from metals such as gold, platinum, iridium, tantalum, or combinations thereof or the distal tip may be made from a polymer containing a radiopaque substance, such as barium sulfate, or the entire antegrade mini-rail shaft 1220 may be radiopaque and/or echogenic.

The antegrade mini-rail shaft 1220 can be constructed of one or more polymers, but may include composites, metals, or combinations thereof. Examples include polyethylene, polyurethane, nylon, Pebax, polyimide, fluoropolymers, etc. The antegrade mini-rail shaft 1220, as well as any part of an antegrade catheter may contain a reinforcement 424, one or more coils, braiding, laminated sections, etc., to enhance the properties as previously described for the rendezvous guide wire shaft 420. Additionally, the antegrade mini-rail shaft 1220 may be constructed to enhance visualization through radiopacity and/or echogenicity as previously described for the rendezvous guide wire shaft 420.

The region of the antegrade mini-rail shaft 1220 that contains the window 990, the window section 1240, may have a different radiopacity or echogenicity than other portions of the mini-rail antegrade catheter 1100. It is preferable to visualize the longitudinal and lateral position of the window 990 as well as rotational orientation of the window 990. FIG. 11A illustrates one embodiment of a mini-rail window radiopaque marker 1260 that provides for longitudinal, lateral, and rotational identification of the window 990. When viewed in the orientation in FIG. 11A, the window 990 can be seen as an antegrade notch 1070 in the mini-rail window radiopaque marker 1260, as well as the semi-circles 1060 can be visualized. When rotated 90 degrees, neither the antegrade notch 1070 is visible as such nor are the full semi-circles 1060.

Other embodiments of a mini-rail window radiopaque marker 1260 can be seen in FIGS. 5C-E with rotational alignment features, or simply a notch 480 without any other additional features.

In other embodiments of the mini-rail window radiopaque marker 1260, the mini-rail window radiopaque marker 1260 can be made of two or more individual radiopaque markers. Both radiopaque markers can be used as described above or one radiopaque marker can be used for identifying orientation in one rotational alignment and the other radiopaque marker for identifying rotational alignment in a different orientation.

The window section 1240 may serve as a transition from the antegrade mini-rail distal region 1120 to the antegrade mini-rail proximal region 1130. The antegrade mini-rail lumen proximal opening 1170 may be located relatively distal to the window 990, the antegrade mini-rail lumen proximal opening 1170 may be located relatively proximal to the window 990, or the antegrade mini-rail lumen proximal opening 1170 may substantially coincide with the window 990.

The window 990 and/or window section 1240 may contain a reinforcement 424, such as with a metal, fiber, or polymer element; braid; coil; safety wire/strap/cable, etc., or made as a separate component and/or integrated into the mini-rail antegrade catheter 1100. This is to provide an additional level of safety that the antegrade mini-rail distal region 1120 and antegrade rendezvous guide wire shaft 1210 never separate. This can also provide for reducing the propensity for the mini-rail antegrade catheter 1100 to deform, buckle, or kink in this region. The window section 1240 reinforcement may be constructed as part of the mini-rail window radiopaque marker 1260 or other longitudinal and/or lateral and/or rotational marker (radiopaque and/or echogenic).

The mini-rail antegrade catheter 1100 contains a window section 1240. An antegrade ramp 1250 may be located near the distal end region of the antegrade rendezvous guidewire lumen 1150 and can be used to direct the rendezvous guide wire 2000 as it passes through the window 990 into and down the antegrade rendezvous guide wire lumen 1150. The antegrade ramp 1250 may be a flat, radiused/curved shape, any configuration that changes the rendezvous guide wire 2000 entry angle to substantially that of the longitudinal axis of the antegrade rendezvous guide wire shaft 1210 and/or antegrade rendezvous guidewire lumen 1150 as the rendezvous guide wire 2000 enters the mini-rail antegrade catheter 1100 through the window 990.

The mini-rail antegrade catheter hub 1200 consists of a Luer fitting or tapered section. The mini-rail antegrade catheter hub 1200 may be attached to the antegrade rendezvous guide wire shaft 1210 such as by bonding or ultrasonic welding, or may be molded onto the antegrade rendezvous guide wire shaft 1210. The mini-rail antegrade catheter hub 1200 is typically plastic, such as polyethylene, polyurethane, polycarbonate, etc., or may be metallic or composite or a combination thereof.

As described for use over a 0.035″ diameter antegrade guide wire, the mini-rail antegrade catheter 1100 is typically 135 cm in working length—from the distal tip to the distal end region of the mini-rail antegrade catheter hub 1200, however, longer or shorter lengths may be constructed depending on the location of the occlusion 2300 and access site. The length of the antegrade mini-rail distal region 1120 is typically 1 cm to 50 cm or longer, more preferably from 4 cm to 20 cm. The length of the window 990 is 0.014″ to 0.8″ or longer, more preferably from 0.040″ to 0.60″, and more preferably from 0.2″ to 0.4″. The overall length of the antegrade mini-rail guide wire lumen 1140 can be from 1 cm to 30 cm or more. More preferably, the overall length of the antegrade mini-rail guide wire lumen 1140 is from 10 cm to 30 cm. The antegrade mini-rail guide wire lumen 1140 is nominally 0.038″ in diameter and may be smaller at the distal tip, e.g. 0.036″, so as to have a tighter fit to the antegrade guide wire 940, however, the antegrade mini-rail guide wire lumen 1140 can be constructed for any guide wire diameter. The antegrade rendezvous guide wire lumen 1150 would be configured for a 0.010″ to 0.018″ rendezvous guide wire 2000, preferably for a 0.014″ rendezvous guide wire 2000 with an inside diameter of 0.017″ to 0.0080″, to facilitate the rendezvous guide wire 2000 entering the window 990 and making the change in direction down the antegrade rendezvous guide wire lumen 1150. The larger the inside diameter, the easier to receive the rendezvous guide wire 2000. The maximum outside diameter of the mini-rail antegrade catheter 1100 is preferably less than or equal to 7 Fr, more preferably less than or equal to 5 Fr.

Portions or all of the mini-rail antegrade catheter 1100 may be coated to enhance reflectivity, increase lubricity, increase stiffness, etc. Examples of coatings include a lubricious coating such as silicone or fluoropolymer, hydrophilic, hydrophobic, etc., on the outside of the antegrade mini-rail shaft 1220 and window section 1240 to reduce the force needed to travel through the vasculature. A polymer surface coating can be applied to the window section 1240 to further increase the reflectivity of that area of the mini-rail antegrade catheter 1100. An internal or external coating (or laminate/layer) of polyimide with a Pebax would increase stiffness and pushability of the antegrade rendezvous guide wire shaft 1210.

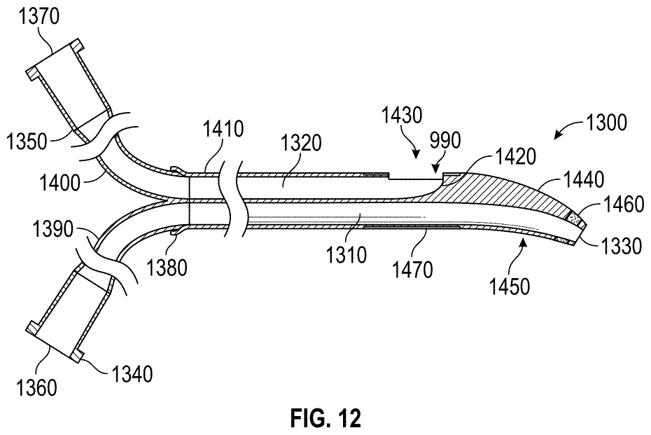

FIG. 12 illustrates an embodiment of a multilumen antegrade catheter in cross-section. The multilumen over the wire (OTW) antegrade catheter 1300 contains two lumens, an antegrade OTW guide wire lumen 1310 and a rendezvous guide wire lumen 1320. The antegrade OTW guide wire lumen 1310 has an antegrade OTW distal opening 1330 and proximally terminates at the antegrade OTW guide wire hub proximal opening 1360 or port. The antegrade OTW rendezvous guide wire lumen 1320 extends from the window section 1430 to the antegrade OTW rendezvous hub proximal opening 1370 or port. Within the antegrade OTW window section 1430, there is a window 990 which serves as an entry point for receiving a rendezvous guide wire 2000 into the multilumen OTW antegrade catheter 1300 with an antegrade OTW window radiopaque marker 1470. The antegrade OTW adapter interface 1380 is where the multilumen OTW antegrade catheter 1300 multilumen shaft region terminates and/or enters a traditional multilumen catheter Y-arm, or splits into pig tails, or suitable adaptor(s), such as the antegrade OTW guide wire pigtail 1390 and antegrade OTW rendezvous pigtail 1400.

The multilumen OTW antegrade catheter 1300 contains a window 990. An antegrade OTW ramp 1420 may be located near the distal end region of the antegrade OTW rendezvous guidewire lumen 1320 and can be used to direct the rendezvous guide wire 2000 as it passes through the window 990 into and down the antegrade OTW rendezvous guide wire lumen 1320. The antegrade OTW ramp 1430 may be a flat, radiused/curved shape, any configuration that changes the rendezvous guide wire 2000 entry angle to substantially that of the longitudinal axis of the antegrade OTW shaft 1410 and/or antegrade OTW rendezvous guidewire lumen 1320 as the rendezvous guide wire 2000 enters the multilumen OTW antegrade catheter 1300 through the window 990.

The antegrade OTW guide wire hub 1340 consists of a Luer fitting or tapered section. The antegrade OTW guide wire hub 1340 may be attached to the antegrade OTW guide wire pigtail 1390 such as by bonding, ultrasonic welding, or may be molded onto the antegrade OTW guide wire pigtail 1390. The antegrade OTW guide wire hub 1340 is typically plastic, such as polyethylene, polyurethane, polycarbonate, etc., or may be metallic or composite or a combination thereof. The antegrade OTW rendezvous hub 1350 consists of a Luer fitting or tapered section. The antegrade OTW rendezvous hub 1350 may be attached to the antegrade OTW rendezvous pigtail 1400 such as by bonding or ultrasonic welding, or may be molded onto the antegrade OTW rendezvous pigtail 1400. The antegrade OTW rendezvous hub 1350 is typically plastic, such as polyethylene, polyurethane, polycarbonate, etc., or may be metallic or composite or a combination thereof. The pigtails are typically a polymer-based in construction and may be laminated, contain braids, or one or more coils.

The antegrade OTW shaft 1410 is constructed to provide for suitable pushability (longitudinal motion) and torquability (rotational motion) such that the multilumen OTW antegrade catheter 1300 can be steered, rotated, and advanced within a vessel 2200 as well as align the window 990 with an exit port 360 of a retrograde catheter. The antegrade OTW shaft 1410 is typically round in cross-section and can be constructed of one or more polymers, composites, metals, or combinations thereof. Examples include polyethylene, polyurethane, nylon, Pebax, polyimide, fluoropolymers, carbon fiber, stainless steel, nitinol, titanium, etc. The antegrade OTW shaft 1410 can be made at least in part as a laminate of various materials. The antegrade OTW shaft 1410 as constructed with allow for lateral motion control as well. The antegrade OTW shaft 1410 may be at least in part constructed to be radiopaque by adding a radiopaque material in a polymer construction, such as barium sulfate or tantalum, or by forming it from a metal or a coated metal (e.g. gold-coated stainless steel), or a combination thereof. Also, the antegrade OTW shaft 1410 may have enhanced echogenicity which may be accomplished by, for example, material selection, surface conditioning and/or surface structures (e.g. dimples, roughing the surface), internal structures or features (e.g. lattice), echogenic coatings, and the like. Radiopacity and echogenicity enable the user to visualize the multilumen OTW antegrade catheter 1300 using fluoroscopy and ultrasound to better improve positioning and orientation.

The antegrade OTW shaft 1410, as well as any part of an antegrade catheter may contain a reinforcement 424, one or more coils, braiding, etc., to enhance the properties. For example, a braid or braided section within the antegrade OTW shaft 1410 would enhance torquability and a coil within antegrade OTW shaft 1410 would enhance pushability. By changing the pitch, material, and number of braids, the torquability (and pushability) can be modified, while changing the spacing on a coil, the pushability and degree of shaft flexibility can be modified. Multiple coils can also be used to enhance torquability. That said, both braids and coils can be used to modify both pushability and torquability and may at least in part be radiopaque.

The antegrade OTW shaft 1410 and/or either antegrade OTW hub may have a visual and/or tactile indicator of the rotational orientation of the window 990 or other more distal multilumen OTW antegrade catheter 1300 element. For example, as previously described, the antegrade OTW rendezvous hub 1340 may have an antegrade OTW hub marker, the antegrade OTW shaft 1410 may have an antegrade OTW shaft marker and/or a stripe as a visual indicator to provide visual feedback and/or raised and/or textured to provide tactile feedback of the rotational orientation of the window 990.

The antegrade OTW distal shaft region 1450 of the multilumen OTW antegrade catheter 1300 contains at least a portion of the antegrade OTW guide wire single lumen shaft 1440 and an antegrade OTW tip radiopaque marker 1460. A portion or the entire length of the antegrade OTW guide wire single lumen shaft 1440 is configured to achieve alignment with a retrograde catheter. The antegrade OTW distal shaft region 1450 may employ one or more curves or bent sections to help steer the catheter through the vasculature as well as position the window 990 against the vessel 2200 wall. The distal tip region of the antegrade OTW distal shaft region 1450 that is curved or bent away from the longitudinal axis of the proximal shaft pushes against the vessel 2200 wall opposite of the side of the window 990, thus pushing the window 990 up to the vessel 2200 wall. To facilitate orientation within the vessel 2200 and alignment with a retrograde catheter and exit port 360, the antegrade OTW distal shaft region 1450 and/or the antegrade OTW window section 1430 configured to be complimentary to the distal shaft region of a retrograde catheter and may be round or non-round shaped such as a flattened, spatulated, crescent shape, edgy, etc. The distal tip or adjacent region of the antegrade OTW distal shaft region 1450 may contain an antegrade OTW tip radiopaque marker 1460, made from metals such as gold, platinum, iridium, tantalum, or combinations thereof or the distal tip may be made from a polymer containing a radiopaque substance, such as barium sulfate, or the entire antegrade mini-rail shaft 1220 may be radiopaque and/or echogenic.

The antegrade OTW window section 1430 may contain a reinforcement 424, such as with a metal, fiber, or polymer element; braid; coil; safety wire/strap/cable, etc., or made as a separate component and/or integrated into the multilumen OTW antegrade catheter 1300. This is to provide for reducing the propensity for the multilumen OTW antegrade catheter 1300 to deform, buckle, or kink in this region. The antegrade OTW window section 1430 reinforcement may be constructed as part of a window radiopaque marker or other longitudinal and/or lateral and/or rotational marker (radiopaque and/or echogenic).

The antegrade OTW window section 1430, may have a different radiopacity or echogenicity than other portions of the multilumen OTW antegrade catheter 1300. It is preferable to visualize the longitudinal and lateral position of the window 990 as well as rotational orientation of the window 990. Referring to FIG. 11A, a similar window radiopaque marker can be employed on the multilumen OTW antegrade catheter 1300. FIG. 11A illustrates one embodiment of a window radiopaque marker 1260 that provides for longitudinal, lateral, and rotational identification of the window 990. When viewed in the orientation in FIG. 11A, the window 990 can be seen as an antegrade notch 1070 in the mini-rail window radiopaque marker 1260, as well as the semi-circles 1060 can be visualized. When rotated 90 degrees, neither the antegrade notch 1070 is visible as such nor are the full semi-circles 1060.

Other embodiments of a mini-rail window radiopaque marker 1260 for use on the multilumen OTW antegrade catheter 1300 can be seen in FIGS. 5C-E with rotational alignment features, or simply a notch 480 without any other additional features.

In other embodiments of a mini-rail window radiopaque marker 1260 for use on the multilumen OTW antegrade catheter 1300, the mini-rail window radiopaque marker 1260 can be made of two or more individual radiopaque markers. Both radiopaque markers can be used as described above or one radiopaque marker can be used for identifying orientation in one rotational alignment and the other radiopaque marker for identifying rotational alignment in a different orientation.

As described for use over a 0.035″ diameter antegrade guide wire, the multilumen OTW antegrade catheter 1300 is typically 135 cm in working length—from the distal tip to the antegrade OTW adapter interface 1380, however, longer or shorter lengths may be constructed depending on the location of the occlusion 2300 and access site. The length of the antegrade OTW guide wire single lumen shaft 1440 is 1 cm to 50 cm or longer, more preferably from 2 cm to 20 cm. The length of the window 990 is 0.014″ to 0.8″ or longer, more preferably from 0.040″ to 0.60″, and more preferably from 0.2″ to 0.4″. The antegrade OTW guide wire lumen 1310 is nominally 0.038″ in diameter or larger (e.g. 5 Fr to 7 Fr) and may be smaller at the distal tip, e.g. 0.036″, so as to have a tighter fit on a 0.035″ antegrade guide wire 940, however, the antegrade OTW guide wire lumen 1310 can be constructed for any guide wire or desired diameter. The antegrade OTW rendezvous guide wire lumen 1320 would be configured for a 0.010″ to 0.018″ rendezvous guide wire 2000, preferably for a 0.014″ rendezvous guide wire 2000 with an inside diameter of 0.017″ to 0.080″, to facilitate the rendezvous guide wire 2000 entering the window 990 and making the change in direction down the antegrade OTW rendezvous guide wire lumen 1320. The larger the inside diameter, the easier to receive the rendezvous guide wire 2000. The maximum outside diameter of the multilumen OTW antegrade catheter 1300 is preferably less than or equal to 7 Fr, more preferably less than or equal to 6 Fr, with an antegrade OTW guide wire single lumen shaft outside diameter of 5 Fr or less at the distal end.

Portions or all of the multilumen OTW antegrade catheter 1300 may be coated to enhance reflectivity, increase lubricity, increase stiffness, etc. Examples of coatings include a lubricious coating such as silicone or fluoropolymer, hydrophilic, hydrophobic, etc., on the outside of the antegrade OTW guide wire single lumen shaft 1440 and antegrade OTW window section 1430 to reduce the force needed to travel through the vasculature. A polymer surface coating can be applied to the antegrade OTW window section 1430 to further increase the reflectivity of that area of the multilumen OTW antegrade catheter 1300. An internal or external coating (or laminate/layer) of polyimide with a Pebax would increase stiffness and pushability of the multilumen OTW antegrade catheter 1300.

To increase the ability to achieve a successful passing of the rendezvous guide wire 2000 from a retrograde catheter into an antegrade catheter, a part or all of the distal regions of antegrade catheter and/or retrograde catheter are form shaped for precise mating and alignment of the antegrade catheter guide wire entry window 990 and the retrograde catheter guide wire exit port 360, e.g. having complimentary surface and radiopacity/echogenicity features.

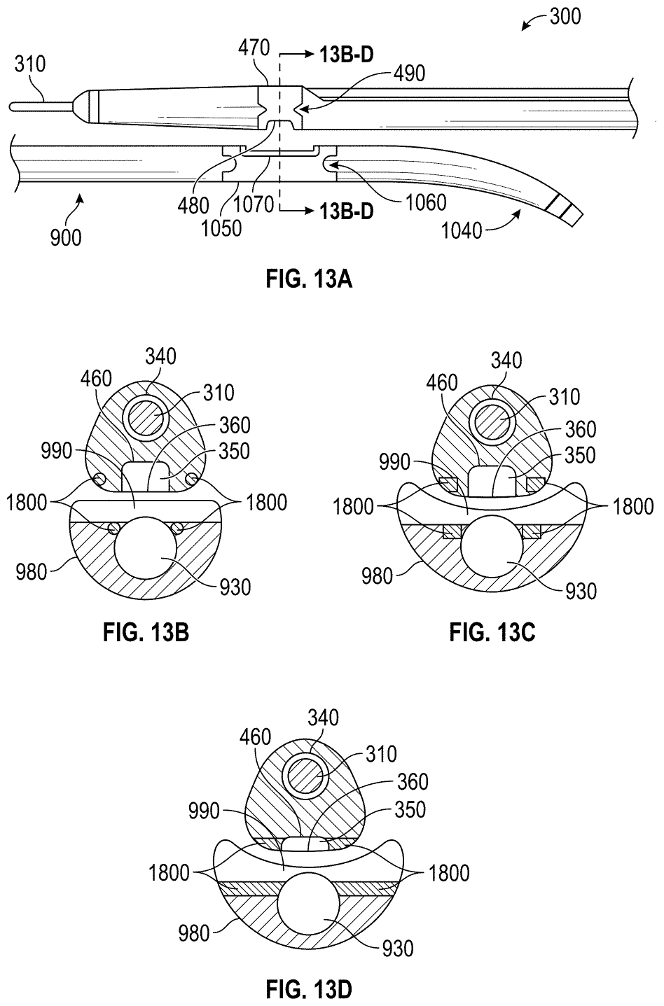

FIG. 13A illustrates a portion of a mini-rail retrograde catheter 300 and a portion of a single lumen antegrade catheter 900 aligned for a passing the rendezvous guide wire 2000 from a retrograde catheter into an antegrade catheter. Similar shapes and features can be employed amongst the various configurations of retrograde and antegrade catheter configurations previously described. The designs enable the window 990 and exit port 360 to easily be aligned rotationally, longitudinally, and laterally.

In one or more embodiments, the retrograde catheter has an exit port radiopaque marker 470 that contains a notch 480 and chevrons 490, while the antegrade catheter has a window radiopaque marker 1050 that has an antegrade notch 1070 and semi-circles 1060. Rotational alignment of both catheters is achieved by rotating each catheter until the notch 480 and chevrons 490 of the exit port radiopaque marker 470 and the antegrade notch 1070 and semi-circles 1060 of the window radiopaque marker 1050 are positioned in the orientation as illustrated in FIG. 13A.

In one or more embodiments, the exit port radiopaque marker 470 is relatively the length of or shorter in length than the window 990 and antegrade notch 1070 where longitudinal alignment is confirmed by radiographic (e.g. fluoroscopy) and/or echogenicity (e.g. ultrasound) by having a retrograde catheter exit port radiopaque marker 470 longitudinally positioned within the antegrade notch 1070 region of an antegrade catheter (FIG. 13A). Longitudinal alignment is accomplished by advancing and/or retracting one or both catheters.

Lateral alignment can be confirmed by taking an orthogonal view from that used for longitudinal alignment and having the exit port radiopaque marker 470 and window radiopaque marker 1050 visually superimposed or stacked on top of each other. Lateral alignment is enhanced by the complimentary features (e.g. cross-section) of one or both of the catheters at least in this region, examples of complimentary features are illustrated in FIGS. 13B-D but not limited to these shapes, as any complimentary features are within the scope of this invention. Adjusting lateral alignment is typically done by changing position of the antegrade catheter, though the retrograde catheter can be repositioned as well. The complimentary features also improve rotational alignment, for example as the catheters come together a flat section of one catheter with try to align with a complimentary flat section of the other catheter.

In one or more embodiments illustrated in FIGS. 13B-D, a retrograde catheter and an antegrade catheter may have magnetic elements 1800 on or within the shaft and/or radiopaque markers that create an attraction between the two catheters to help pull them together when in close proximity, to improve lateral and rotational alignment, as well as longitudinal alignment, and improve stability of the two catheters during tissue manipulation (dissection) and/or during passage of the rendezvous guide wire 2000. Close proximity of the window 990 and exit port 360 is desirable to minimize the distance the rendezvous guide wire 200 has to travel outside of a catheter, where the rendezvous guide wire 2000 would have a chance to change direction outside of the exit port 360 and not be directed into the window 990. The magnetic elements 1800 may be of various shapes, sizes, strengths, and in various locations depending on the degree of attraction required. The window radiopaque marker 1050 and exit port radiopaque marker 470 may be magnetic and serve this purpose.

In one or more embodiments, the exit port 360 region of a retrograde catheter may be magnetic and one or more of the antegrade distal shaft region 1040, antegrade distal tip 1080, antegrade tip radiopaque marker 1030, and/or dissection feature 1090 may be magnetic to assist in pulling the dissection feature 1090 up to the exit port 360 region and against any tissue, to assist in creating a dissection, micro-dissection, tissue disruption, or passage through any tissue (e.g. intima 2220) that may be present between the vessel lumen 2210 and the exit window 360 of a retrograde catheter.

In one or more embodiments, close proximity of the window 990 and exit port 360 is enhanced by having one or more curves or bent sections in the antegrade distal shaft region 1040 where the distal portion of an antegrade catheter pushes against the vessel 2200 wall opposite of the side of the window 990, thus pushing the window 990 up towards the exit port 360. This along with the complimentary shapes, improves lateral and rotational alignment. Close proximity of the window 990 an exit port 360 is desirable to minimize the distance the rendezvous guide wire 200 has to travel outside of a catheter, where the rendezvous guide wire 2000 would have a chance to change direction outside of the exit port 360 and not be directed into the window 990.

In one or more embodiments the antegrade catheter lumen that is to receive the rendezvous guide wire 2000 is larger in diameter than the retrograde catheter lumen that is used to deliver the rendezvous guide wire 2000 and the window 990 is wider than the exit port 360. For example, using a 0.014″ rendezvous guide wire 2000, the retrograde catheter lumen that is used to deliver the rendezvous guide wire 2000 is 0.017″ in diameter, while the antegrade catheter lumen that is to receive the rendezvous guide wire 2000 is 0.038″ in diameter. This, along with the window 990 being longer than the exit port 360, provides for achieving successful rendezvous guide wire 2000 delivery from the retrograde catheter into the antegrade catheter with some degree of rotational, longitudinal, or lateral misalignment.

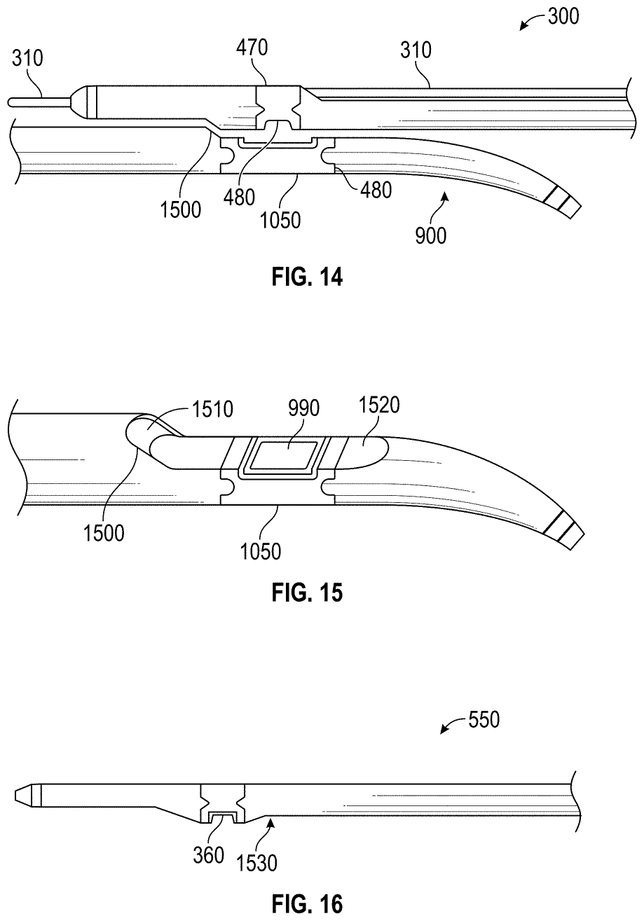

In one or more embodiments, the retrograde catheter and/or the antegrade catheter may have one or more features or design elements such as a step, bend, bumper, etc., that is used to enhance longitudinal, and/or lateral and/or rotational alignment. FIG. 14 illustrates a portion of a mini-rail retrograde catheter 300 and a portion of a single lumen antegrade catheter 900 with a step 1500 feature aligned for advancing the rendezvous guide wire 2000 from the retrograde catheter into the antegrade catheter. The step 1500 engages the angle of the mini-rail retrograde catheter 300 and provides for longitudinal alignment, and where there is a complimentary surface on the retrograde catheter, rotational and lateral alignment as well. The step 1500 may have a pocket 1510 or recess (FIG. 15) that provides for longitudinal, rotational, and/or lateral alignment. This can be accomplished by having a complimentary feature on the retrograde catheter that engages the recess 1510 or where the retrograde catheter is smaller in width than the antegrade catheter, the retrograde catheter can laterally fit into the recess 1510. Also illustrated is a flat 1520 complimentary feature.

In one or more embodiments, the retrograde catheter and/or the antegrade catheter may have one or more features or design elements to provide for the retrograde catheter rendezvous guide wire lumen to be in communication with an antegrade rendezvous guide wire lumen, such as by aligning the exit port 360 and window 990 either in direct contact or spaced apart, enabling advancement of a (rendezvous) guide wire through the exit port 360 and the window 990. Thus, the first and second side ports are in “communication” with each other as long as they are close enough in proximity and axial and rotational alignment that a wire exiting the exit port 360 can enter the window 990 to accomplish the crossing described herein.

One example of a feature or design element to improve positioning is an offset portion of the catheter shaft such that the exit port 360 and/or window 990 are directed towards the other catheter. FIG. 16 illustrates an over the wire retrograde catheter 550 with an offset 1530 section of OTWR multilumen shaft 660 in the region of the exit port 360. This offset 1530 section of OTWR multilumen shaft 660 can be used to position the exit port 360 closer to a window 990 and can also be used as a feature for longitudinal, lateral, and rotational alignment. The offset 1530 section may also contain one or more complimentary features, such as the flat 1520 illustrated in FIG. 15. Such an offset section could be on either the antegrade or retrograde catheter or both.

In one or more embodiments, the retrograde catheter and/or the antegrade catheter may have one or more features or design elements such as an extendable/retractable member(s) or element(s) which extend away from the catheter shaft and can be used to move the exit port 360 and/or window 990 in a direction, generally towards the other catheter/towards each other. This feature(s) or design element(s) could be a single narrow element or could be up to one half or three quarters or more of the way around the catheter to actively push the exit port 360 and/or window 990 towards the other catheter/towards each other. Examples of extendable/retractable elements follow, but the invention is not limited to these and any element that can actively move the catheter away from the vessel 2200 wall and/or towards the other catheter is within the scope of this invention.

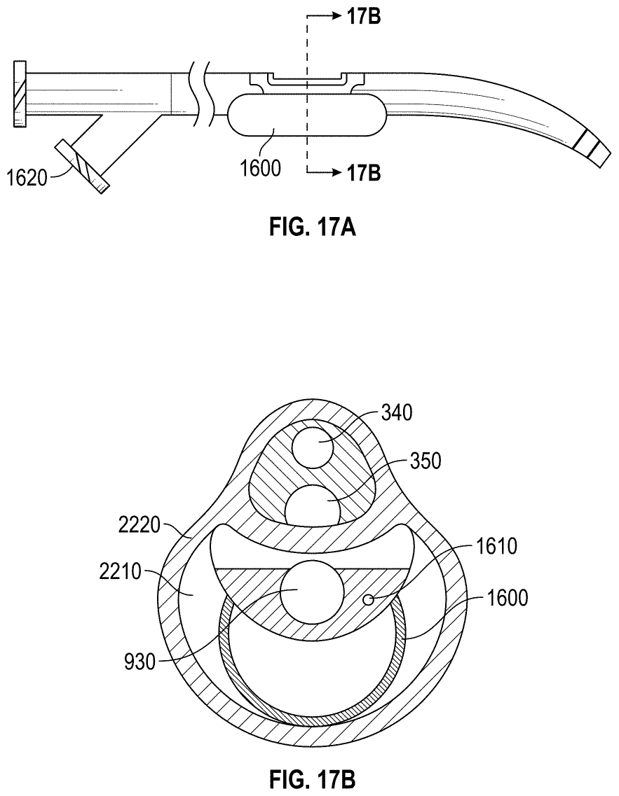

In one or more embodiments, the extendable/retractable element is a balloon 1600. FIG. 17A depicts a balloon 1600 on a single lumen antegrade catheter 900. The balloon 1600 is primarily on the opposite side of the single lumen antegrade catheter 900 as the window 990, such that when inflated, the balloon 1600 moves the window 990 region preferably towards a retrograde catheter. FIG. 17B illustrates a cross-section through a single lumen antegrade catheter 900, the inflated balloon 1600, the vessel 2200, vessel lumen 2210, and a retrograde catheter. As can be seen with the balloon 1600 inflated, the window 990 is moved adjacent the exit port 360. Actively pushing the antegrade and retrograde catheters together will assist in alignment as previously described, for example a flat and the slight crescent shape or flat with wings will be urged to go parallel for rotational orientation and also orient laterally due to the retrograde catheter flat feature centering within the antegrade catheter flat with wings feature. To inflate the balloon 1600, an additional balloon inflation lumen 1610, and a balloon inflation port 1620 in the proximal region of the catheter will be required.

The balloon 1600 can be narrow or wide, shaped, long or short, just so long as it moves the catheter as intended. A balloon 1600 can be added to and used on any of the retrograde and/or antegrade catheters of the invention. Multiple balloons 1600 can be used. The balloon 1600 can typically be made from various polymers, such as polyethylene, polyurethane, nylon, etc.

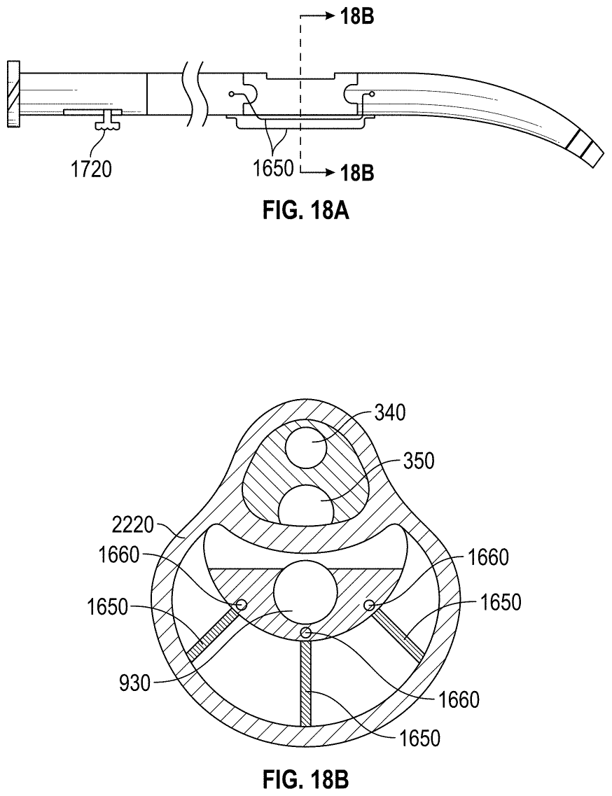

In one or more embodiments, the extendable/retractable element is one or more extension wire(s) 1650 that extends away from the catheter shaft. FIG. 18A depicts three extension wires 1650 extended or deployed on a single lumen antegrade catheter 900. The extension wires 1650 are primarily on the opposite side of the single lumen antegrade catheter 900 as the window 990, so as when extended, the extension wires 1650 move the window 990 region preferably towards a retrograde catheter. FIG. 18B illustrates a cross-section through a single lumen antegrade catheter 900, the three extension wires 1650 extended, the vessel 2200, vessel lumen 2210, and a retrograde catheter. As can be seen with the extension wires 1650 extended, the window 990 is moved adjacent the exit port 360. Actively pushing the antegrade and retrograde catheters together will assist in alignment as previously described, for example a flat and the slight crescent shape or flat with wings will be urged to go parallel for rotational orientation and orient laterally due to the retrograde catheter flat feature centering within the antegrade catheter flat with wings feature. To extend the extension wire(s) 1650, they are moved out (and retracted) by use of an extension wire actuator 1720 in the proximal region of the catheter. An extension wire lumen(s) 1660 will contain the extension wire(s) 1650 within the catheter where they are not deployed.

The extension wire(s) 1650 can be narrow or wide, shaped, long or short, just so long as it/they moves the catheter as intended. Extension wire (s) 1650 can be made from metals, e.g. stainless steel, nitinol, tungsten, etc., or other materials with sufficient properties to move the catheter. Extension wire(s) 1650 can be added to and used on any of the retrograde and/or antegrade catheters of the invention.

FIGS. 17B and 18B both illustrate a thin section of vascular tissue between a retrograde catheter and an antegrade catheter. This may be the case or the exit port 360 of a retrograde catheter may be in the vessel lumen 2210 without any tissue between the exit port 360 of a retrograde catheter and the window 990 of an antegrade catheter.

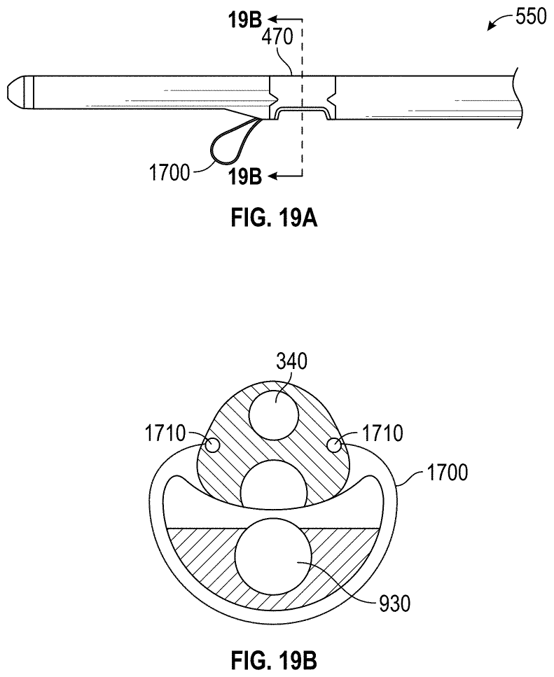

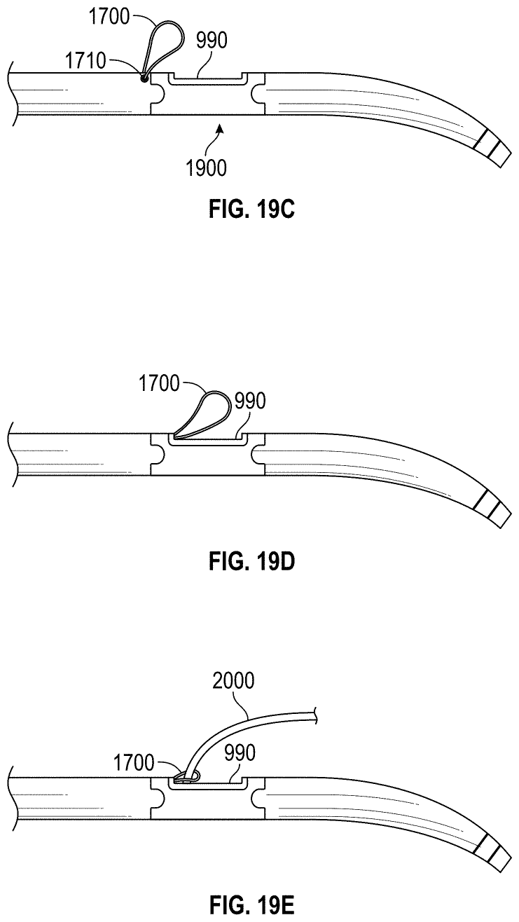

In one or more embodiments, the retrograde catheter and/or the antegrade catheter may have one or more features or design elements such as an extendable/retractable member(s) or element(s) to grab or engage the other catheter, such as a hoop 1700 or loop. FIG. 19A depicts a hoop 1700 extended or deployed from an over the wire retrograde catheter 550. The hoop 1700 is primarily on the same side of the over the wire retrograde catheter 550 as the exit port 360, so as when extended, the hoop 1700 moves out in to the vessel lumen 2210 to enable capture or guiding of the antegrade catheter. FIG. 19B illustrates a cross-section through an over the wire retrograde catheter 550 with hoop 1700 and an antegrade catheter captured in the hoop 1700. As can be seen with the hoop 1700 capturing the antegrade catheter, the antegrade catheter and window 990 is positioned adjacent the exit port 360. Actively moving the antegrade and retrograde catheters together will assist in alignment as previously described, for example a flat and the slight crescent shape or flat with wings will be urged to go parallel for rotational orientation and orient laterally due to the retrograde catheter flat feature centering within the antegrade catheter flat with wings feature. To extend the hoop 1700, the hoop 1700 is moved out (and retracted) by use of one or more pull wires or cables and a hoop actuator in the proximal region of the catheter. The hoop 1700 may be fully extended out to the vessel 2200 wall such that the antegrade catheter easily moves through the hoop 1700, and then the hoop 1700 can be partially retracted capturing the antegrade catheter as illustrated in FIG. 19B. One or more hoop lumens 1710 will contain the pull wire(s) or cable(s) within the catheter.

FIGS. 19C-E illustrate a portion of an antegrade catheter adapted to incorporate a hoop 1700. In this configuration, the antegrade catheter with hoop 1900, allows for the hoop 1700 to be extended and used to capture the rendezvous guide wire 2000, retract/retrieve the rendezvous guide wire 2000 to enable pulling or guiding the rendezvous guide wire 2000 through the window 990, capturing a retrograde catheter, etc. The hoop 1700 can extend from antegrade catheter with hoop 1900 proximally, distally (illustrated in FIG. 19C), or from within the window 990 (illustrated in FIG. 19D) or window region. In FIG. 19C, the hoop 1700 extends from two hoop lumens 1710 to interact with the rendezvous guide wire 2000 and/or a retrograde catheter. As previously described when positioned on a retrograde catheter, the hoop 1700 can assist in actively moving the antegrade catheter with hoop 1900 and retrograde catheter together. In this configuration, the hoop 1700 is used in a similar fashion as previously described. The hoop 1700 can also be used to capture the rendezvous guide wire 2000 and then by retracting the antegrade catheter with hoop 1900, bring the rendezvous guide wire 2000 back out of the antegrade access site 910 to complete the rendezvous guide wire 2000 positioning within the patient.

FIGS. 19D-E illustrate the hoop 1700, extending from two hoop lumens 1710 located withing the window 990 region. This allows using the hoop actuator to extend the hoop 1700 in order to capture the rendezvous guide wire 2000 (FIG. 19D) and draw the rendezvous guide wire 2000 into the window 990 (FIG. 19E). Once drawn into the window 990, the hoop 1700 can release the rendezvous guide wire 2000 and the rendezvous guide wire 2000 can be advanced through the antegrade catheter with hoop 1900 until it extends out of the patient or antegrade catheter with hoop 1900. Alternately, the hoop 1700 can continue to capture the rendezvous guide wire 2000 and the entire antegrade catheter with hoop 1900 can be retracted out of the antegrade access site 910.

The hoop 1700 and/or pull wire(s) or cable(s) can typically be made from metals, e.g. stainless steel, nitinol, tungsten, etc., polymers, fibers, or other materials with sufficient properties to form the hoop 1700 and move the hoop 1700. A hoop 1700 can be added to and used on any of the retrograde and/or antegrade catheters of the invention.

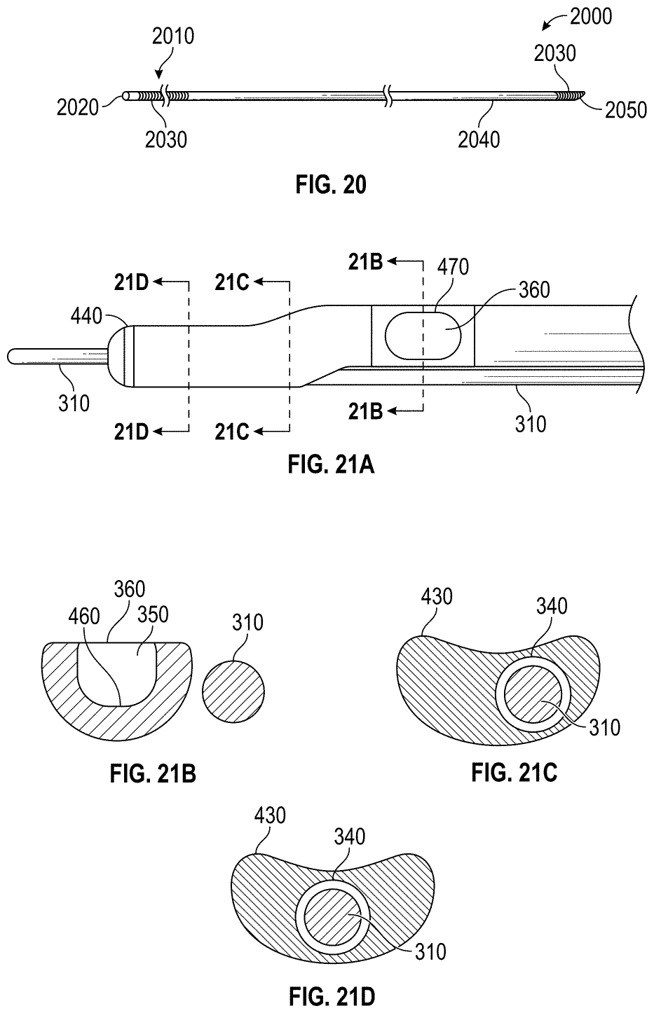

As illustrated in FIG. 20, the rendezvous guide wire 2000 has a distal end region 2010 and a proximal end region 2040. The distal end region 2010 includes coils 2030 and/or a jacket (e.g. polymer coating) over a core wire component. Any portion or all of the rendezvous guide wire 2000 may be coated, such as with a lubricious coating (e.g. fluoropolymer or silicone based, hydrophilic or hydrophobic). The distal end 2020 is generally atraumatic, unless it incorporates a piercing tip, as will be described on the proximal end region 2040. The coils 2030 may be constructed of one or more sets of coils 2030, for example, a radiopaque section of coils 2030 near the distal tip and a non-radiopaque section of coils 2030 proximally adjacent the distal radiopaque coils 2030. The coils 2030 may be spaced apart or stacked to varying degrees in varying places along the one or more coils 2030. The rendezvous guide wire 2000 may be constructed with an end region (distal or proximal) configured to ease the transition from a retrograde catheter guide wire lumen and exit port 360 into the window 990 and a lumen of an antegrade catheter that is used for the rendezvous guide wire 2000. This may be accomplished by having a very soft (floppy) distal end region with a gradual transition to a stiffer section in the proximal direction. A long taper on a core wire or varying coils (shape, diameter, materials, etc.), or materials (e.g. nitinol, stainless steel, polymeric), or combinations thereof may be used to achieve the desired flexibility and pushability. An example is a core wire with a main length diameter of 0.013″. The central stainless steel section is attached to a distal nitinol section, that includes a long taper (e.g. 10 cm) down to 0.0015″ in diameter. The 0.0015″ section is 2 cm in length and may be or contain a portion that is flattened. The nitinol section has a more proximal coil constructed of a 0.0025″ diameter stainless steel wire, with a 10% spacing, with an outside diameter of the coil being 0.013″. The nitinol section has a more distal coil constructed of a 0.002″ diameter platinum wire, with a 25% spacing, with an outside diameter of the coil being 0.013″. The distal tip is rounded to be atraumatic. The proximal side of the stainless steel core wire is tapered over a 5 cm length down to 0.003″. The 0.003″ section is 1 cm in length. Over the proximal taper, there is a coil constructed of a 0.003″ diameter platinum wire, with a 25% spacing, with an outside diameter of the coil being 0.013″. The entire rendezvous guide wire 2000 is coated with a hydrophilic coating up to an outside diameter from 0.0135″ to 0.0140″.

The proximal end region 2040 may include a proximal piercing tip 2050 or may be atraumatic (e.g. rounded) at the end. The proximal end region 2040 may also include a tapered core wire and/or separate element and coils 2030 similar to the distal end region 2010. The proximal piercing tip 2050 is designed such that it can penetrate tissue easier than a rounded tip, examples include pointed, faceted, angled, beveled, etc. The proximal piercing tip 2050 (or if the piercing tip is located on the distal end) may be used to pierce any tissue that is present between a retrograde catheter exit port 360 and the antegrade catheter window 990 or the vessel lumen 2210. This may be done with or without an antegrade catheter aligned and in place for advancing the rendezvous guide wire 2000 from the retrograde catheter into the antegrade catheter.

Retrograde guide wire 310 size influences the needle 100 size as well as the guide wire lumen size of a retrograde catheter. The size of the rendezvous guide wire 2000 influences both catheter selections, while the size of the antegrade guide wire 940 influences an antegrade catheter size. As has been described, a typical selection would be to use a 0.014″ retrograde guide wire 310 and associated needle 100, with a 0.014″ rendezvous guide wire 2000 as this is typically placed in a smaller diameter section of the vessel 2200 and it is preferable to have a smaller cross-sectional area of the retrograde catheter region that is to cross the occlusion 2300. The antegrade catheter is generally not crossing the occlusion 2300 so it can be larger in cross-section and use a larger diameter antegrade guide wire 940, such as a 0.035″ guide wire. These may be paired to have the desired complimentary features, such as window 990/exit port 360 lengths, window radiopaque marker 1050 and exit port radiopaque marker 470, surfaces and features like a step 1500, flat 1520, recess 1510, and/or offset 1530, distal curves or bends, stiffness, and overall catheter length depending on access site location. Rendezvous guide wire 2000 length is typically 260 cm to 300 cm.

Illustrated in FIGS. 21A-D is an alternate embodiment of the mini-rail retrograde catheter 300 as illustrated in FIGS. 4A-D. In this embodiment, the shaft/lumens of the side-by-side mini-rail retrograde catheter 520 are effectively rotated 90 degrees with respect to each other. This side-by-side configuration places the ramp 460 pointing approximately 90 degrees from a plane bisecting the mini-rail guide wire lumen 340 and rendezvous guide wire lumen 350. In this configuration, there is less radial distension of the vessel 2200 wall compared to the mini-rail retrograde catheter 300 of FIG. 4A. The distal region may be configured to enable dissection through a vessel 2200 wall (e.g. between the intima and media) and/or occlusion; examples include round or non-round shaped such as a flattened, spatulated, crescent shape, edgy, etc.; to facilitate orientation within the vessel 2200; alignment with an antegrade catheter; and passage across occluded segments allowing blunt or cutting dissection similar to surgical elevators (in effect, a remotely introduced endovascular surgical tool).

In one or more embodiments, this side-by-side configuration can be used on any of the catheters of the present invention that have more than one lumen for a guide wire.

In one or more embodiments, the distal region of the antegrade and retrograde catheters can have one or more bends or curves with respect to the longitudinal axis of the proximal shaft of the catheter.

In one or more embodiments, the antegrade and retrograde catheters may align within the occluded segment or occlusion 2300, cranial to (above) the occluded segment (as described), caudal to (below) the occluded segment, medial and lateral, or anterior and posterior. As such, an antegrade catheter may have any or all of the features, components, and design aspects to partially or wholly cross an occlusion 2300 previously described for a retrograde catheter, and a retrograde catheter may have any or all of the features, components, and design aspects to align with an antegrade catheter enabling a rendezvous guide wire 2000 to be passed through the catheters in any location with respect to the occlusion 2300.

The RampTech System 10 and/or individual components or any subset of the components described herein can be used as a complete system, individually, in combinations, and/or with other needles, guide wires, catheters, and vascular and non-vascular devices. Various sizes and combinations can be selected and used depending upon the intended clinical procedure.

### Example Procedure (Method)

The following example describes one procedure for treating a lower limb total occlusion with the present invention, but the apparatus and techniques can be used in any vascular and non-vascular location where there is access to both sides of an occlusion 2300 or target region. This example describes creating a directional dissection plane in the intima of the vessel 2200 wall around the occlusion 2300, the apparatus and techniques will also work through any portion of the vessel 2200 wall, occlusion 2300, and/or within the true lumen of the vessel 2200. This example is for illustrative purposes only, and is not intended to describe the numerous procedural and device combination alternatives that are contemplated within the scope of the present invention.

### Obtain Retrograde Access

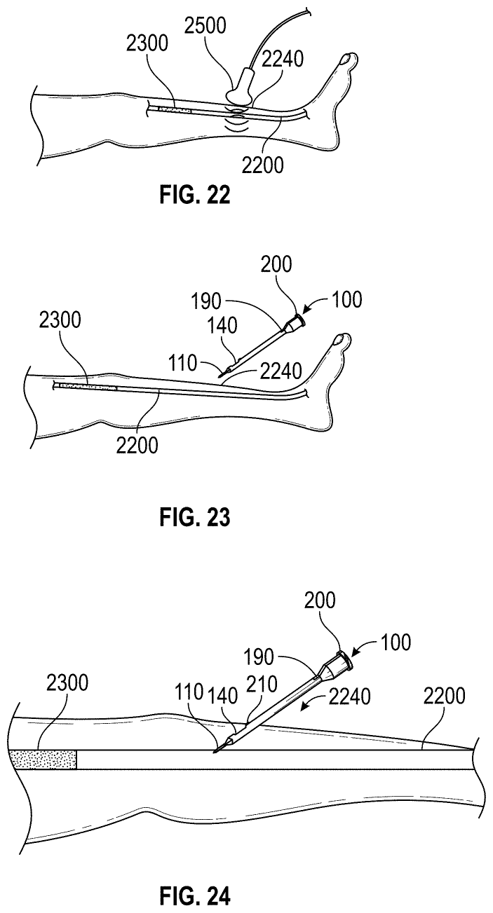

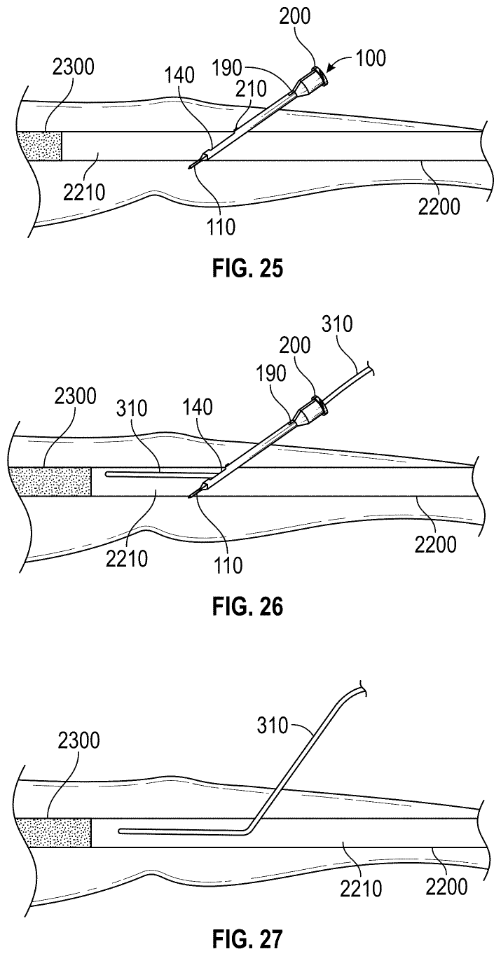

Identify and locate the target retrograde vessel 2200 (distal to the occlusion 2300), e.g. pedal, tibal artery, using fluoroscopy, an ultrasound probe 2500, visual, and/or pressure techniques. FIG. 22. Determine the retrograde access site 2240 for needle 100 insertion into the selected vessel 2200, preferably distal of the target occlusion 2300. FIG. 23. Orient the needle 100 using the needle shaft marker 190 and/or needle hub marker 200 for rotational orientation, such that the needle distal lumen opening 140 is pointed in the direction intended for the guide wire and retrograde catheter to advance, i.e. in the retrograde direction to flow in the vessel 2200. Position the needle 100 at the desired angle of entry into the tissue and vessel 2200, such as a 37±20 degree angle to the vessel 2200, though the design of the needle 100 allows for even more shallow angles, such as 10 to 17 degrees. Insert the lance tip 110 portion of the needle 100 into the tissue and then continue advancing the needle 100 until the lance tip 110 enters the target vessel lumen 2210. FIG. 24. Carefully continue advancing the needle 100 until there is visible flashback of blood through the needle 100, or slight resistance is felt, or other indicator that the needle distal lumen opening 140 is within the vessel lumen 2210, the lance tip 110 has gone through the opposite side of the vessel 2200, and the bumper 210 has reached the entry side of the vessel 2200. FIG. 25. Various ways to confirm that the needle distal lumen opening 140 is within the vessel lumen 2210 can be employed, such as observation of blood backflow, aspirating through the needle 100 and looking for blood, contrast injection through the needle 100, etc. Slight manipulation of the needle 100 may be required to confirm/optimize the needle distal lumen opening 140 position within the vessel lumen 2210.

With appropriate needle 100 position confirmed, advance a 0.014″ retrograde guide wire 310 or other guide wire through the needle 100 and into the vessel lumen 2210. FIG. 26. Either 0.010″ or 0.018″ guide wires may also be used, as long as the appropriate needle 100 for that diameter guide wire is/are used. Once the 0.014″ retrograde guide wire 310 or other guide wire is sufficiently advanced into the vessel lumen 2210, while holding the 0.014″ retrograde guide wire 310 or other guide wire securely in position, carefully retract the needle 100 out of the patient and off of the 0.014″ retrograde guide wire 310 or other guide wire. This leaves the 0.014″ retrograde guide wire 310 or other guide wire positioned in the vessel lumen 2210. FIG. 27. A microcatheter 250 may now be placed in the vessel 2200 to maintain access if the retrograde guide wire 310 is in place, or to establish a lumen though which the other guide wire may be removed and the retrograde guide wire 310 inserted, or to enlarge the passageway through/around/past an occlusion 2300, or the procedure may directly move to introducing a retrograde catheter over the 0.014″ retrograde guide wire 310 without use of a microcatheter 250.

### Introduce Retrograde Catheter and Cross Occlusion

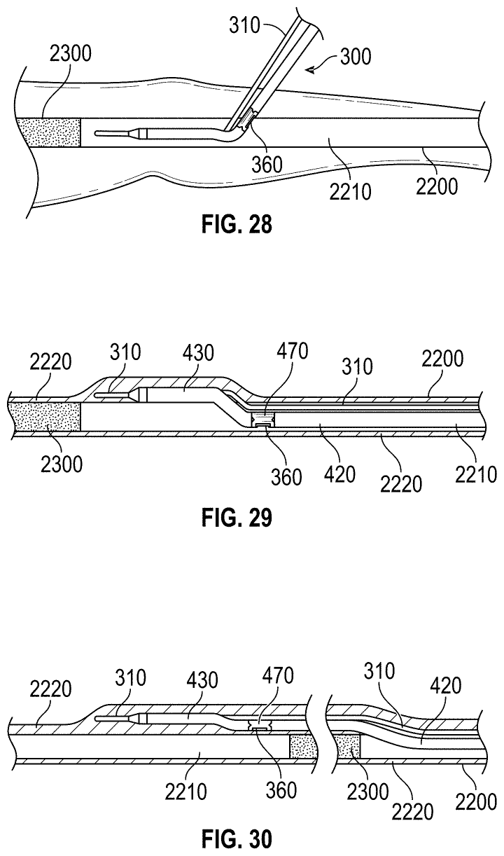

The distal end of the retrograde catheter, illustrated is this example is a mini-rail retrograde catheter 300, is now loaded onto the 0.014″ retrograde guide wire 310 proximal end and advanced up to the tissue, though any of the retrograde catheters of the invention can be used. While securely holding the 0.014″ retrograde guide wire 310 in place, the mini-rail retrograde catheter 300 is advanced through the tissue and into the vessel lumen 2210. FIG. 28. The 0.014″ retrograde guide wire 310 alone or with the mini-rail retrograde catheter 300 together as a unit are advanced up to the occlusion 2300. The 0.014″ retrograde guide wire 310 alone or with the mini-rail retrograde catheter 300 together as a unit are advanced slightly which either directs the 0.014″ retrograde guide wire 310 and mini-rail retrograde catheter 300 into the vessel 2200 wall (e.g. subintimal, within the intima 2220) or in a position to go around (within the vessel lumen 2210) or through the occlusion 2300. The mini-rail retrograde catheter 300 is rotationally oriented using exit port radiopaque marker 470 and/or visual and or tactile markers (e.g. hub marker 370) to position the exit port 360 facing towards the vessel lumen 2210, preferably towards the centerline of the vessel lumen 2210. FIG. 29. The mini-rail retrograde catheter 300 is then advanced until the exit port 360 is located on the other side of the occlusion 2300 with the exit port 360 facing towards the vessel lumen 2210, preferably towards the centerline of the vessel lumen 2210. The physical design (shape, flexibility, hardness, material, etc.) of the mini-rail retrograde catheter 300 enhances the ability to orient and create a directional dissection plane while advancing to the opposite side of the occlusion 2300. FIG. 30. Of note, the antegrade and retrograde catheters may align within the occluded segment or occlusion 2300, cranial to (above) occluded segment, or caudal to (below) occluded segment, medial and lateral, or anterior and posterior.

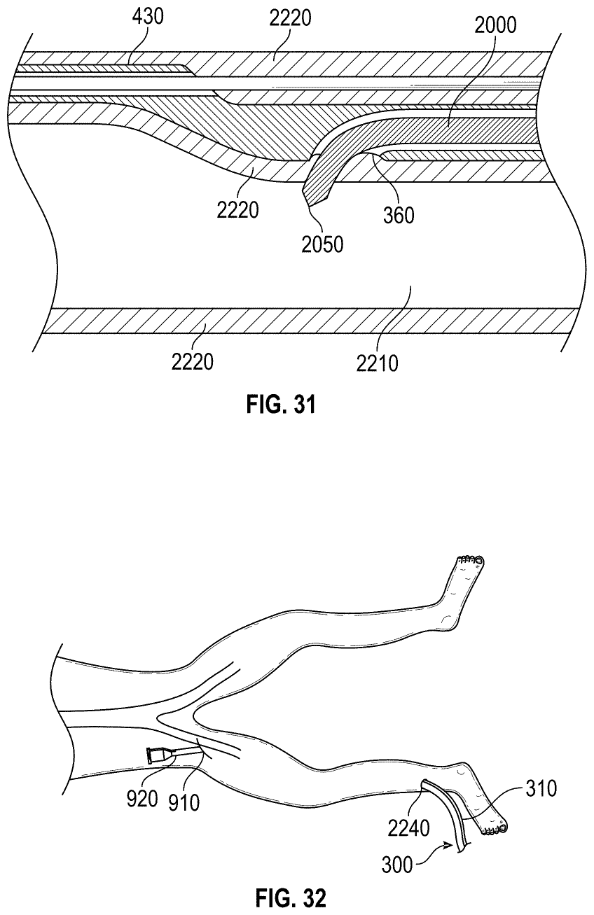

If desirable, this step can be conducted at this time or after an antegrade catheter is introduced and/or in position. The rendezvous guide wire 2000 may be introduced through the mini-rail retrograde catheter hub 410 of the mini-rail retrograde catheter 300 and into the rendezvous guide wire lumen 350 with the proximal end region 2040 going in first. In this example, a 0.014″ rendezvous guide wire 2000 will be described, but the rendezvous guide wire 2000 can be of any size. The 0.014″ rendezvous guide wire 2000 is advanced through the rendezvous guide wire lumen 350 until it reaches the ramp 460. The proximal piercing tip 2050 of the rendezvous guide wire 2000 may be used to penetrate any tissue (e.g. intima 2220) between the mini-rail retrograde catheter 300 and the antegrade catheter window 990 and/or vessel lumen 2210. FIG. 31. Once the tissue is penetrated, the rendezvous guide wire 2000 may be retracted out of the mini-rail retrograde catheter 300.

### Establish Antegrade Access

Identify and locate the target antegrade vessel 2200 (proximal to the occlusion 2300), e.g. femoral or contralateral femoral artery, radial artery, etc., using fluoroscopy, ultrasound, visual, and/or pressure techniques. Using typical cutdown or percutaneous (e.g. Seldinger) technique, access the target vessel 2200 at the antegrade access site 910 with a needle. FIG. 32. Carefully advance the needle until there is visible flashback of blood through the needle or other sign confirming appropriate needle position, such as aspirating through the needle and looking for blood, contrast injection through the needle, etc. Slight manipulation of the needle 100 may be required to confirm position. With appropriate needle position confirmed, advance an 0.035″ antegrade guide wire 940 through the needle and into the vessel lumen 2210. Any guide wire appropriately sized for the antegrade catheter may be used, from 0.014″ up to 0.038″ guide wires or larger may also be used, as long as the appropriate needle for that diameter guide wire is used. In this example, a single lumen antegrade catheter 900 will be used that is designed for a 0.014″ rendezvous guide wire 2000 and a 0.035″ antegrade guide wire 940. Once the 0.035″ antegrade guide wire 940 is sufficiently advanced into the vessel lumen 2210, while holding the 0.035″ antegrade guide wire 940 securely in position, carefully retract the needle out of the patient and off of the 0.035″ antegrade guide wire 940. This leaves the 0.035″ antegrade guide wire 940 positioned in the vessel lumen 2210. An antegrade introducer sheath 920 may now be placed in the vessel 2200 to maintain access as illustrated or the procedure may directly move to introducing the single lumen antegrade catheter 900 over the 0.035″ antegrade guide wire 940.

### Introduce Antegrade Catheter

The distal end of an antegrade catheter, illustrated is this example is a single lumen antegrade catheter 900 but any of the antegrade catheters of the invention can be used, is now loaded onto the proximal end of the 0.035″ antegrade guide wire 940 and advanced up to the tissue. While securely holding the 0.035″ antegrade guide wire 940 in place, the single lumen antegrade catheter 900 is advanced through the tissue and into the vessel lumen 2210. The 0.035″ antegrade guide wire 940 alone or with the single lumen antegrade catheter 900 together as a unit are advanced up to the region of the vessel 2200 where the distal region 320 of the mini-rail retrograde catheter 300 is located.

Dissect Tissue with Antegrade Catheter

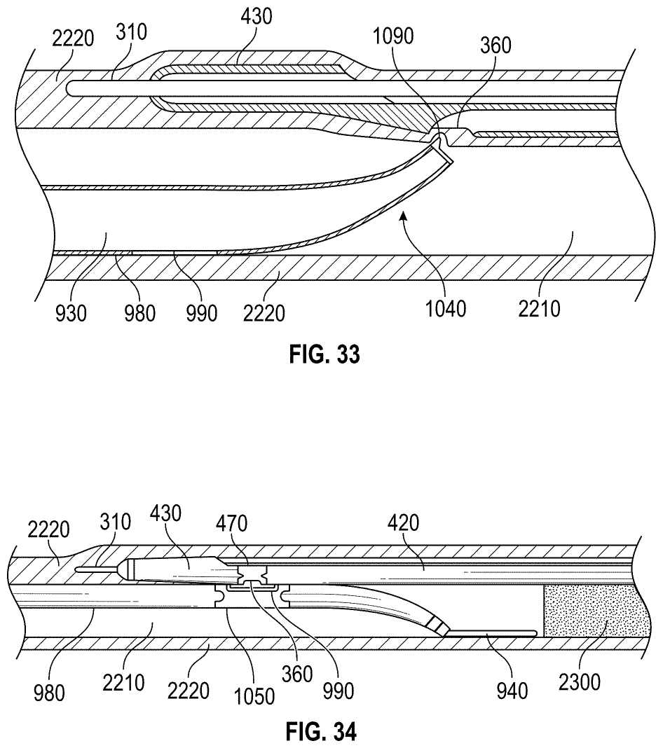

If desirable, the antegrade distal tip 1080 of an antegrade catheter may be rotated and aligned such that it is in contact with or pointing towards the vessel 2200 wall at the location of the retrograde catheter exit port 360. The antegrade guide wire 940 is retracted and/or positioned to achieve the desired deflection of the antegrade distal shaft region 1040 to engage the dissection feature 1090 against the vessel 2200 wall. The antegrade catheter, antegrade distal shaft region 1040, and/or the dissection feature 1090 are manipulated (e.g. moving longitudinally, laterally, a combination thereof) to disrupt any tissue (e.g. intima 2220) between the exit port 360 and the vessel lumen 2210. This procedure may be conducted with just the antegrade distal tip 1080 of an antegrade catheter without a dissection feature 1090. FIG. 33.

Align Antegrade Catheter with Retrograde Catheter

The single lumen antegrade catheter 900 is rotationally oriented using window radiopaque marker 1050 and/or semi-circles 1060 and/or antegrade notch 1070 and/or visual and/or tactile markers as previously described on the retrograde catheter to position the window 990 in rotational alignment with the exit port 360 of the mini-rail retrograde catheter 300. The single lumen antegrade catheter 900 is then fully advanced until features as previously described on one or both catheters enable rotational, longitudinal, and lateral alignment of the exit port 360 and the window 990. FIG. 34. The mini-rail retrograde catheter 300 illustrated has a spatulated distal region 320 that orients with a complimentary surface of the single lumen antegrade catheter 900. Various complimentary surfaces can be implemented as previously described. In addition, radiopaque alignment markers on both catheters improve fluoroscopic identification of proper alignment. It can be seen that the slight curvature or offset in the distal region of the single lumen antegrade catheter 900 assists in pushing the window 990 towards the mini-rail retrograde catheter 300. Additionally, the offset (or a curvature) of the distal region 320 of the mini-rail retrograde catheter 300 assists in positioning the exit port 360 towards the window 990. Both passive and active features may be employed to assist in alignment/positioning/capture as described previously.

Of note, antegrade and retrograde catheters may align within the occluded segment or occlusion 2300, cranial to (above) occluded segment (as illustrated), or caudal to (below) occluded segment, or medial and lateral, or anterior and posterior.

In addition, when aligned or not aligned, movement of the antegrade catheter and retrograde catheter individually or in combination with each other may be employed to create a channel through the occlusion. The channel is then used to pass a guide wire or catheter.

### Rendezvous Guide Wire

The step of piercing any tissue between the mini-rail retrograde catheter 300 and the antegrade catheter window 990 and/or the vessel lumen 2210, if not previously conducted, such as described and illustrated in FIG. 31, or by the act of moving both antegrade and retrograde catheters, may now, if desirable, be conducted.

With the exit port 360 and the window 990 and their respective lumens in communication with each other, e.g. aligned and positioned towards each other either in direct contact or spaced apart, the rendezvous guide wire 2000, distal end region 2010 first, is introduced through the mini-rail retrograde catheter hub 410 of the mini-rail retrograde catheter 300 and into the rendezvous guide wire lumen 350. In this example, a 0.014″ rendezvous guide wire 2000 will be described, but the rendezvous guide wire 2000 can be of any size. The 0.014″ rendezvous guide wire 2000 is advanced through the rendezvous guide wire lumen 350 until it reaches the ramp 460.

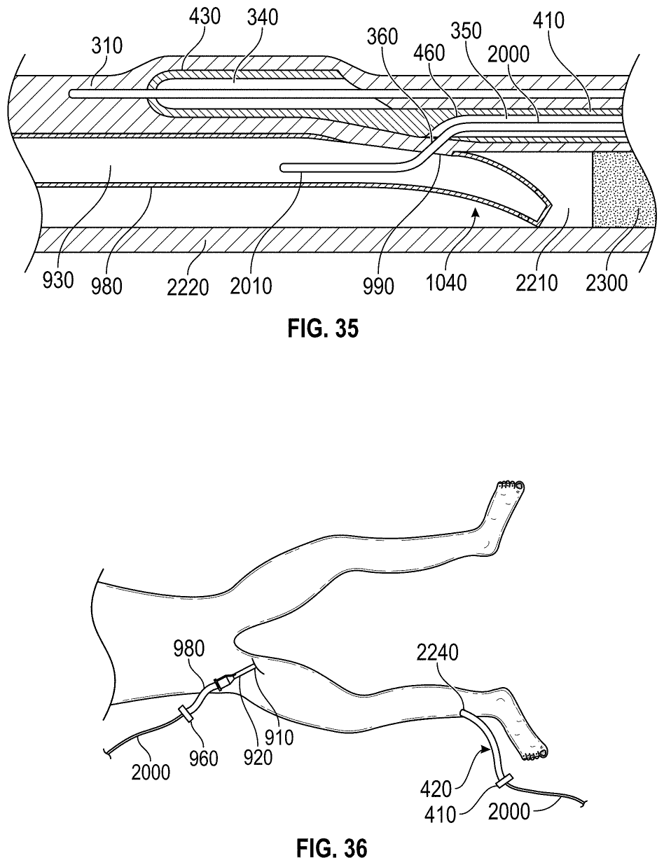

If the single lumen antegrade catheter 900 is an over the wire design as illustrated, the antegrade 0.035″ guide wire is retracted out of the single lumen antegrade catheter 900 (this may be done earlier in the process). Proper orientation of the catheters is confirmed. The 0.014″ rendezvous guide wire 2000 is advanced with the ramp 460 deflecting the 0.014″ rendezvous guide wire 2000 out the exit port 360 at an angle. The 0.014″ rendezvous guide wire 2000 enters the single lumen antegrade catheter 900 window 990 and into the antegrade single lumen 930 (over the wire design) or antegrade catheter rendezvous guide wire lumen 1150 (mini-rail and multilumen designs). To achieve this, the 0.014″ rendezvous guide wire 2000 may have to pass through a portion of the vessel 2200 wall (typically intima 2220/endothelium) before entering the window 990. FIG. 35. The angle at which the 0.014″ rendezvous guide wire 2000 exits the exit port 360 is influenced by the ramp 460 design (curvature, angle, length, etc.) and enhances the ability of the 0.014″ rendezvous guide wire 2000 to pass through and tissue/vessel 2200 wall and enter the window 990 and antegrade single lumen 930 and be advanced through the entire length of the single lumen antegrade catheter 900 until a portion of the 0.014″ rendezvous guide wire 2000 exits the antegrade single lumen hub 960. FIG. 36.

The “rendezvous” guidewire 2000 as described herein refers to a specialized guide wire having structural features and properties as have been described in connection with FIG. 31 and elsewhere herein. However, any guidewire with sufficient length and suitable diameter and flexibility may be used to achieve through and through guidewire placement.

### Remove Antegrade and Retrograde Catheters

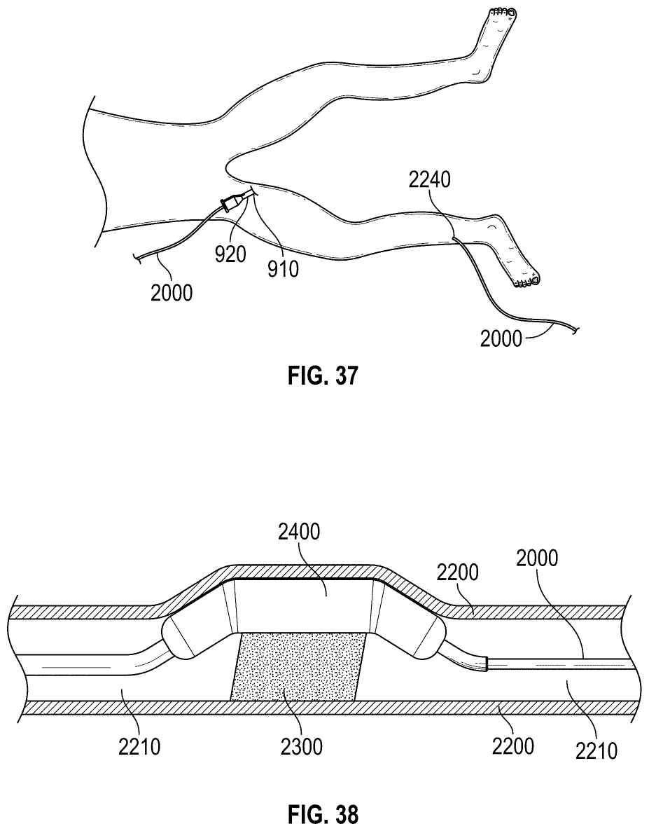

This can be done in either order. Holding the 0.014″ rendezvous guide wire 2000 in position, retract the single lumen antegrade catheter 900 out of the patient such that the 0.014″ rendezvous guide wire 2000 is accessible from the antegrade access site 910. Holding the 0.014″ rendezvous guide wire 2000 in position, retract the mini-rail retrograde catheter 300 out of the patient such that the 0.014″ rendezvous guide wire 2000 is accessible from the retrograde access site 2240. This leaves the 0.014″ rendezvous guide wire 2000 across the target occlusion 2300 and exiting the patient from both access sites. FIG. 37.

### Perform Dilatation/Stenting Procedure

The 0.014″ rendezvous guide wire 2000 may now be used as part of a balloon dilatation catheter 2400 or similar revascularization device procedure and may include placement of one or more stents, tacks, etc., to open a lumen in the vessel 2200 and provide for restoration/increased blood flow through the vessel 2200 through/around/past the occlusion 2300. FIG. 38 illustrates and balloon dilatation catheter 2400 inserted from the antegrade access site 910 over the 0.014″ rendezvous guide wire 2000 in position and creating a pathway for blood flow, such as by dilating the vessel 2200/intima 2220 and potentially compressing the occlusion 2300. Blood flow through the vessel 2200 may be confirmed using fluoroscopy and contrast injection, ultrasound, or typical methods.

### Completion of the Procedure

Once blood flow through/around/past the occlusion 2300 is sufficiently restored, the balloon dilatation catheter 2400 or similar revascularization device is removed from the vessel 2200 and the 0.014″ rendezvous guide wire 2000 is removed from the vessel 2200. These may be removed individually or as a single unit. Both access sites are closed using any of the typical access site closure procedures, e.g. closure devices, compression, etc.

### EXAMPLE EMBODIMENTS

An access needle for introducing a wire into a vessel comprising one or more of the following:
an elongate body having a proximal end, a distal end and a longitudinal axis;
a lumen extending between an opening on the proximal end and a side port on the body; and
a stabilizer extending distally from the distal end.

An access needle as disclosed in any embodiment herein, comprising a transition between the body and the stabilizer.

An access needle as disclosed in any embodiment herein, wherein the outside diameter or major axis/cross-section length in a non-round embodiment of the stabilizer is no more than about 70% of the diameter of the body.

An access needle as disclosed in any embodiment herein, further comprising a bumper on the body, proximal to the side port.

A retrograde catheter for introduction into a vascular lumen and retrograde advancement to a treatment site, comprising one or more of the following:
an elongate, flexible tubular body having a proximal end and a distal end;
a first lumen extending between a proximal opening adjacent the proximal end and a side port spaced proximally apart from the distal end; and
a second lumen extending proximally from the distal end to a second lumen proximal port.

A retrograde catheter as disclosed in any embodiment herein, wherein the distal region is configured to enable dissection through a vessel wall.

A retrograde catheter as disclosed in any embodiment herein, comprising the retrograde catheter configured to facilitate alignment with an antegrade catheter.

A retrograde catheter as disclosed in any embodiment herein, further comprising the retrograde catheter configured to facilitate alignment of the retrograde catheter side port with an antegrade catheter side port.

A retrograde catheter as disclosed in any embodiment herein, comprising a radiopaque marker to indicate the location of the side port.

A retrograde catheter as disclosed in any embodiment herein, comprising fluoroscopically visible indicium of rotational orientation.

A retrograde catheter for introduction into a vascular lumen and retrograde advancement to a treatment site, comprising one or more of the following:
an elongate, flexible tubular body having a proximal end and a distal end;
a central lumen extending between a proximal opening adjacent the proximal end and the distal end;
a side port spaced proximally apart from the distal end and in communication with the central lumen; and
a moveable ramp adjacent the side port.

An antegrade catheter for accessing a vascular lumen, comprising one or more of the following:
an elongate, flexible tubular body, having a proximal end, a distal end, and a central lumen extending from adjacent the proximal end to a distal exit port;
a side port spaced proximally apart from the distal exit port and in communication with the central lumen;
wherein at least a portion of the tubular body in between the side port and the distal port is not aligned with the longitudinal axis of the proximal region of the tubular body.

An antegrade catheter as disclosed in any embodiment herein, wherein at least a portion of the tubular body in between the side port and the distal port is pre curved, defining a concave side of the catheter, and the side port is circumferentially offset from the concave side of the catheter.

An antegrade catheter as disclosed in any embodiment herein, comprising a second lumen extending proximally from the distal end to a second lumen proximal port.

An antegrade catheter as disclosed in any embodiment herein, further comprising the antegrade catheter configured to facilitate alignment with the retrograde catheter.

An antegrade catheter as disclosed in any embodiment herein, further comprising the antegrade catheter configured to facilitate alignment of the retrograde catheter side port with the antegrade catheter side port.

An antegrade catheter as disclosed in any embodiment herein, further comprising a radiopaque marker to indicate the location of the side port.

An antegrade catheter as disclosed in any embodiment herein, further comprising fluoroscopically visible indicium of rotational orientation.

An antegrade catheter as disclosed in any embodiment herein, further comprising the antegrade catheter having a dissection element on the distal end.

An antegrade catheter as disclosed in any embodiment herein, further comprising the antegrade catheter having a relatively larger outside diameter at the side port and a relatively smaller outside diameter at the distal end.

An antegrade catheter for accessing a vascular lumen, comprising one or more of the following:
an elongate, flexible tubular body having a proximal end and a distal end;
a first lumen extending between a proximal opening adjacent the proximal end and a side port spaced proximally apart from the distal end; and
a second lumen extending proximally from the distal end to a second lumen proximal port.
wherein at least a portion of the tubular body in between the side port and the distal port is not aligned with the longitudinal axis of the proximal region of the tubular body.

A guide wire for accessing a vascular lumen, comprising one or more of the following:
a flexible core member; and
a piercing tip.

A method of crossing a vascular obstruction in a patient, comprising one or more of the following steps:
advancing a first catheter transvascularly in a first direction towards a vascular obstruction, the first catheter having a first central lumen in communication with a first side port;
advancing a second catheter transvascularly in a second, opposite direction towards the obstruction, the second catheter having a second central lumen in communication with a second side port;
aligning the first and second side ports to place the first central lumen in communication with the second central lumen; and
advancing a wire through the first and second side ports such that a first end of the wire is on a first side of the obstruction and a second end of the wire is on a second side of the obstruction.

A system for crossing a vascular occlusion and restoring blood flow, comprising one or more of the following:
a retrograde catheter, having a proximal end, a distal end, a first central lumen and a first side port spaced proximally apart from the distal end, wherein the first central lumen ends distally at the first side port or communicates with and extends distally beyond the first side port; and
an antegrade catheter, having a proximal end, a distal end, a second central lumen and a second side port spaced proximally apart from the distal end, wherein the second central lumen is in communication with the second side port of the antegrade catheter.

A method of bidirectional crossing of a vascular obstruction in a patient, comprising one or more of the steps of:
advancing a first catheter transvascularly in a first direction towards a vascular obstruction, the first catheter having a first central lumen in communication with a first side port;
advancing a second catheter transvascularly in a second, opposite direction towards the obstruction, the second catheter having a second central lumen in communication with a second side port;
aligning the first and second side ports to place the first central lumen in communication with the second central lumen; and
advancing a wire through the first and second side ports such that a first end of the wire is on a first side of the obstruction and a second end of the wire is on a second side of the obstruction.

A method of bidirectional crossing of a vascular obstruction as disclosed in any embodiment herein, wherein the advancing a first catheter step comprises advancing the first catheter in a retrograde direction from an access site.

A method of bidirectional crossing of a vascular obstruction as disclosed in any embodiment herein, wherein the advancing a first catheter step comprises advancing the first catheter beyond the vascular obstruction prior to the aligning step.

A method of bidirectional crossing of a vascular obstruction as disclosed in any embodiment herein, comprising passing the first catheter through vascular tissue to bypass the obstruction.

A method of bidirectional crossing of a vascular obstruction as disclosed in any embodiment herein, wherein the advancing a wire step comprises advancing a wire through the first catheter, through the first and second side ports, through the second catheter and out of the patient.

A method of bidirectional crossing of a vascular obstruction as disclosed in any embodiment herein, further comprising removing the first and second catheters from the patient, leaving the wire in position across the obstruction.

A method of bidirectional crossing of a vascular obstruction as disclosed in any embodiment herein, further comprising guiding a revascularization device along the wire and restoring flow across the obstruction.

A system for crossing a vascular occlusion and restoring blood flow as disclosed in any embodiment herein, further comprising an access needle configured for accessing a vessel.

A system for crossing a vascular occlusion and restoring blood flow as disclosed in any embodiment herein, further comprising a wire having a tissue piercing tip.

A system for crossing a vascular occlusion and restoring blood flow as disclosed in any embodiment herein, wherein the retrograde catheter further comprises a third lumen extending proximally from the distal end to a third lumen proximal port.

A system for crossing a vascular occlusion and restoring blood flow as disclosed in any embodiment herein, wherein the retrograde catheter third lumen proximal port is spaced distally apart from the proximal end.

A system for crossing a vascular occlusion and restoring blood flow as disclosed in any embodiment herein, wherein a distal region on the retrograde catheter is configured to enable dissection through a vessel wall.

A system for crossing a vascular occlusion and restoring blood flow as disclosed in any embodiment herein, further comprising an alignment feature on the retrograde catheter configured to facilitate alignment with the antegrade catheter.

A system for crossing a vascular occlusion and restoring blood flow as disclosed in any embodiment herein, wherein the alignment feature is configured to facilitate alignment of the retrograde catheter side port with the antegrade catheter side port.

A system for crossing a vascular occlusion and restoring blood flow as disclosed in any embodiment herein, further comprising a radiopaque marker on the retrograde catheter to indicate the location of the side port.

A system for crossing a vascular occlusion and restoring blood flow as disclosed in any embodiment herein, wherein the retrograde catheter further comprises a fluoroscopically visible indicium of rotational orientation.

A system for crossing a vascular occlusion and restoring blood flow as disclosed in any embodiment herein, wherein the antegrade catheter further comprises a fourth lumen extending proximally from the distal end to a fourth lumen proximal port.

A system for crossing a vascular occlusion and restoring blood flow as disclosed in any embodiment herein, further comprising an alignment feature on the antegrade catheter configured to facilitate alignment with the retrograde catheter.

A system for crossing a vascular occlusion and restoring blood flow as disclosed in any embodiment herein, wherein the alignment feature is configured to facilitate alignment of the retrograde catheter side port with the antegrade catheter side port.

A system for crossing a vascular occlusion and restoring blood flow as disclosed in any embodiment herein, wherein the antegrade catheter further comprises a radiopaque marker to indicate the location of the side port.

A system for crossing a vascular occlusion and restoring blood flow as disclosed in any embodiment herein, wherein the antegrade catheter further comprises a fluoroscopically visible indicium of rotational orientation.

A system for crossing a vascular occlusion and restoring blood flow as disclosed in any embodiment herein, wherein a proximal region of the antegrade catheter has a first longitudinal axis and a distal region of the antegrade catheter has a second longitudinal axis that is laterally offset from the first longitudinal axis.

A system for crossing a vascular occlusion and restoring blood flow as disclosed in any embodiment herein, further comprising the antegrade catheter having a dissection element on the distal end.

A system for crossing a vascular occlusion and restoring blood flow as disclosed in any embodiment herein, wherein the antegrade catheter further comprises a relatively larger outside circumference at the side port and a relatively smaller outside circumference at the distal end.

1. A system for crossing a vascular occlusion and restoring blood flow within a vessel, the system comprising:
a retrograde catheter being configured to be inserted into the vessel and comprising:
a first proximal end,
a first distal end,
a first side port being spaced proximally apart from the first distal end,
a first central lumen extending proximally from the first side port and towards the first proximal end and being in fluid communication the first side port, and
a guidewire lumen being in a distal region of the retrograde catheter, the guidewire lumen being offset from a longitudinal axis of the retrograde catheter; and
an antegrade catheter being configured to be inserted into the vessel and comprising:
a second proximal end,
a hub on the second proximal end,
a second distal end,
a second side port on a window region spaced proximally apart from the second distal end, and
a second central lumen extending between the hub and the second side port and being in fluid communication with the second side port.

2. A system as in claim 1, further comprising an access needle configured for accessing the vessel.

3. A system as in claim 1, further comprising a wire having a tissue piercing tip.

4. A system as in claim 1, wherein the retrograde catheter further comprises a third lumen extending proximally from the first distal end to a third lumen proximal port.

5. A system as in claim 4, wherein the third lumen proximal port is spaced distally apart from the first proximal end.

6. A system as in claim 1, wherein the distal region of the retrograde catheter is configured to enable dissection through a vessel wall.

7. A system as in claim 1, wherein the retrograde catheter further comprises an alignment feature being configured to facilitate alignment with the antegrade catheter.

8. A system as in claim 7, wherein the alignment feature is configured to facilitate alignment of the first side port with the second side port.

9. A system as in claim 1, wherein the retrograde catheter further comprises a radiopaque marker being configured to indicate a location of the first side port.

10. A system as in claim 1, wherein the retrograde catheter further comprises a fluoroscopically visible indicium of rotational orientation.

11. A system as in claim 1, wherein the antegrade catheter further comprises a fourth lumen extending proximally from the second distal end to a fourth lumen proximal port.

12. A system as in claim 1, wherein the antegrade catheter further comprises an alignment feature being configured to facilitate alignment with the retrograde catheter.

13. A system as in claim 12, wherein the alignment feature is configured to facilitate alignment of the first side port with the second side port.

14. A system as in claim 1, wherein the antegrade catheter further comprises a radiopaque marker to indicate a location of the second side port.

15. A system as in claim 1, wherein the antegrade catheter further comprises a fluoroscopically visible indicium of rotational orientation.

16. A system as in claim 1, wherein a proximal region of the antegrade catheter has a first longitudinal axis and wherein a distal region of the antegrade catheter has a second longitudinal axis that is laterally offset from the first longitudinal axis.

17. A system as in claim 1, wherein the antegrade catheter further comprises a dissection element on the second distal end.

18. A system as in claim 1, wherein the antegrade catheter further comprises a first outside circumference at the second side port and a second outside circumference at the second distal end, and wherein the first outside circumference is larger than the second outside circumference.

19. A system as in claim 1, wherein the window region comprises a non-circular transverse cross section.
